SEQUENCE LISTING (16 sequences)

SEQ ID NO: 1 - DNA, 2189 bp, Homo sapiens
  CDS (435)..(1391)
  sig_peptide (435)..(509)
  mat_peptide (510)..(1391)
ggcacgaggatgcggggaggctgcgtgtgtgcgcagggagagaacgcggccaccttcccgcttccgagctgggtgcgcgccgagcacaggagattgcctgcgtttaggaggtggctgcgttgtgggaaaagctatcaaggaagaaattgccaaaccatgtctttttttctgttttcagagtagttcacaacagatctgagtgttttaattaagcatggaatacagaaaacaacaaaaaacttaagctttaatttcatctggaattccacagttttcttagctccctggacccggttgacctgttggctcttcccgctggctgctctatcacgtggtgctctccgactactcaccccgatgtaaagaaccttcggctcgcgtgcttctgagcggctgtggatggcctcggtctctggactgtccttccgagtaggatgtcactgagatccctcaaatggagcctcctgctgctgtcactcctgagtttctttgtgatgtggtacctcagccttccccactacaatgtgatagaacgcgtgaactggatgtacttctatgagtatgagccgatttacagacaagactttcacttcacacttcgagagcattcaaactgctctcatcaaaatccatttctggtcattctggtgacctcccacccttcagatgtgaaagccaggcaggccattagagttacttggggtgaaaaaaagtcttggtggggatatgaggttcttacatttttcttattaggccaagaggctgaaaaggaagacaaaatgttggcattgtccttagaggatgaacaccttctttatggtgacataatccgacaagattttttagacacatataataacctgaccttgaaaaccattatggcattcaggtgggtaactgagttttgccccaatgccaagtacgtaatgaagacagacactgatgttttcatcaatactggcaatttagtgaagtatcttttaaacctaaaccactcagagaagtttttcacaggttatcctctaattgataattattcctatagaggattttaccaaaaaacccatatttcttaccaggagtatcctttcaaggtgttccctccatactgcagtgggttgggttatataatgtccagagatttggtgccaaggatctatgaaatgatgggtcacgtaaaacccatcaagtttgaagatgtttatgtcgggatctgtttgaatttattaaaagtgaacattcatattccagaagacacaaatcttttctttctatatagaatccatttggatgtctgtcaactgagacgtgtgattgcagcccatggcttttcttccaaggagatcatcactttttggcaggtcatgctaaggaacaccacatgccattattaacttcacattctacaaaaagcctagaaggacaggatactttgtggaaagtgttaaataaagtaggtactgtggaaaattcatggggaggtcagtgtgctggcttacactgaactgaaactcatgaaaaacccagactggagactggagggttacacttgtgatttattagtcaggcccttcaaagatgatatgtggaggaattaaatataaaggaattggaggtttttgctaaagaaattaataggaccaaacaatttggacatgtcattctgtagactagaatttcttaaaagggtgttactgagttataagctcactaggctgtaaaaacaaaacaatgtagagttttatttattgaacaatgtagtcacttgaaggttttgtgtatatcttatgtggattaccaatttaaaaatatatgtagttctgtgtcaaaaaacttcttcactgaagttatactgaacaaaattttacctgtttttggtcatttataaagtacttcaagatgttgcagtatttcacagttattattatttaaaattacttcaactttgtgtttttaaatgttttgacgatttcaatacaagataaaaaggatagtgaatcattctttacatgcaaacattttccagttacttaactgatcagtttattattgatacatcactccattaatgtaaagtcataggtcattattgcatatcagtaatctcttggactttgttaaatattttactgtggtaatatagagaagaattaaagcaagaaaatctgaaaaaaaaaaaaaaaaaaaaa

SEQ ID NO: 2 - protein, 319 aa, Homo sapiens
MSLRSLKWSLLLLSLLSFFVMWYLSLPHYNVIERVNWMYFYEYEPIYRQDFHFTLREHSNCSHQNPFLVILVTSHPSDVKARQAIRVTWGEKKSWWGYEVLTFFLLGQEAEKEDKMLALSLEDEHLLYGDIIRQDFLDTYNNLTLKTIMAFRWVTEFCPNAKYVMKTDTDVFINTGNLVKYLLNLNHSEKFFTGYPLIDNYSYRGFYQKTHISYQEYPFKVFPPYCSGLGYIMSRDLVPRIYEMMGHVKPIKFEDVYVGICLNLLKVNIHIPEDTNLFFLYRIHLDVCQLRRVIAAHGFSSKEIITFWQVMLRNTTCHY

SEQ ID NO: 3 - DNA, 1271 bp, Homo sapiens
  CDS (47)..(1102)
  sig_peptide (47)..(130)
  mat_peptide (131)..(1102)
ggggctgggccctgctcaggtggctctctccttgcagggaccggcgatgctctgcaggctgtgctggctggtctcgtacagcttggctgtgctgttgctcggctgcctgctcttcctgaggaaggcggccaagcccgcagagaccccacggcccaccagcctttctggggctcccccaacaccccgtcacagccggtgtccacccaaccacacagtgtctagcgcctctctgtccctgcctagccgtcaccgtctcttcttgacctatcgtcactgccgaaatttctctatcttgctggagccttcaggctgttccaaggataccttcttgctcctggccatcaagtcacagcctggtcacgtggagcgacgtgcggctatccgcagcacgtggggcaggtggggggatgggctagggccggcactgaagctggtgttcctcctaggggtggcaggatccgctcccccagcccagctgctggcctatgagagtagggagtttgatgacatcctccagtgggacttcactgaggacttcttcaacctgacgctcaaggagctgcacctgcagcgctgggtggtggctgcctgcccccaggcccatttcatgctaaagggagatgacgatgtctttgtccacgtccccaacgtgttagagttcctggatggctgggacccagcccaggacctcctggtgggagatgtcatccgccaagccctgcccaacaggaacactaaggtcaaatacttcatcccaccctcaatgtacagggccacccactacccaccctatgctggtgggggaggatatgtcatgtccagagccacagtgcggcgcctccaggctatcatggaagatgctgaactcttccccattgatgatgtctttgtgggtatgtgcctgaggaggctggggctgagccctatgcaccatgctggcttcaagacatttggaatccggcggcccctggaccccttagacccctgcctgtatagggggctcctgctggttcaccgcctcagccccctcgagatgtggaccatgtgggcactggtgacagatgaggggctcaagtgtgcagctggccccataccccagcgctgaagggtgggttgggcaacagcctgagagtggactcagtgttgattctctatcgtgatgcgaaattgatgcctgctgctctacagaaaatgccaacttggttttttaactcctctcaccctgttagctctgattaaaaacactgcaacccaaaaaaaaaaaaaaaaaa

SEQ ID NO: 4 - protein, 352 aa, Homo sapiens
MLCRLCWLVSYSLAVLLLGCLLFLRKAAKPAETPRPTSLSGAPPTPRHSRCPPNHTVSSASLSLPSRHRLFLTYRHCRNFSILLEPSGCSKDTFLLLAIKSQPGHVERRAAIRSTWGRWGDGLGPALKLVFLLGVAGSAPPAQLLAYESREFDDILQWDFTEDFFNLTLKELHLQRWVVAACPQAHFMLKGDDDVFVHVPNVLEFLDGWDPAQDLLVGDVIRQALPNRNTKVKYFIPPSMYRATHYPPYAGGGGYVMSRATVRRLQAIMEDAELFPIDDVFVGMCLRRLGLSPMHHAGFKTFGIRRPLDPLDPCLYRGLLLVHRLSPLEMWTMWALVTDEGLKCAAGPIPQR

SEQ ID NO: 5 - protein, 325 aa, Homo sapiens
MQSKHRKLLLRCLLVLPLILLVDYCGLLTHLHELNFERHFHYPLNDDTGSGSASSGLDKFAYLRVPSFTAEVPVDQPARLTMLIKSAVGNSRRREAIRRTWGYEGRFSDVHLRRVFLLGTAEDSEKDVAWESREHGDILQADFTDAYFNNTLKTMLGMRWASEQFNRSEFYLFVDDDYYVSAKNVLKFLGRGRQSHQPELLFAGHVFQTSPLRHKFSKWYVSLEEYPFDRWPPYVTAGAFILSQKALRQLYAASVHLPLFRFDDVYLGIVALKAGISLQHCDDFRFHRPAYKGPDSYSSVIASHEFGDPEEMTRVWNECRSANYA

SEQ ID NO: 6 - DNA, 337 bp, Homo sapiens
  misc_feature (18): n equals a, t, g or c
  misc_feature (101): n equals a, t, g or c
  misc_feature (158): n equals a, t, g or c
  misc_feature (168): n equals a, t, g or c
  misc_feature (279): n equals a, t, g or c
ccaaaaaacccatatttnttaccaggagtatcctttcaaggtgttccctccatactgcagtgggttgggttatataatgtccagagatttggtgccaaggntctatgaaatgatgggtcacgtaaaacccatcaagtttgaagatgtttatgtcgggntctgtttgantttattaaaagtgaacattcatattccagaagacacaaatcttttctttctatatagaatccatttggatgtctgtcaactgagacgtgtgattgcagcccatggcttttnttccaaggagatcatcactttttggcaggtcatgcttaagggaacaccacatgccatt

SEQ ID NO: 7 - DNA, 246 bp, Homo sapiens
  misc_feature (104): n equals a, t, g or c
aattcggcacgagccactacccaccctatgctggtgggggaggatatgtcatgtccagagccacagtgcggcgcctccaggctatcatggaagatgctgaactnttccccattgatgatgtctttgtgggtatgtgcctgaggaggctggggctgagccctatgcaccatgctggcttcaagacatttggaatccggcggcccctggaccccttagacccctgcctgtatagggggctcctgctgg

SEQ ID NO: 8 - DNA, 356 bp, Homo sapiens
  misc_feature (5): n equals a, t, g or c
  misc_feature (8): n equals a, t, g or c
  misc_feature (318): n equals a, t, g or c
  misc_feature (327): n equals a, t, g or c
  misc_feature (346): n equals a, t, g or c
  misc_feature (348): n equals a, t, g or c
  misc_feature (351): n equals a, t, g or c
  misc_feature (356): n equals a, t, g or c
ggcanagncttagcccatcctcaggggagggaggactctggaaagagcctgaggatacgctgagaataaaaagtccagaggccagcagggacttagcggctggggtgcaggttaactcctgcccaactcttggggacagggaacccggggctgggccctgctcaggtggctctctccttgcagggaccggcgaatgctctgcaggctgtgctggctggtctcgtacagcttggcttgctgttgctcggctgcctgctacttcctgaggaaggcggccaagcccgcagagaaccccacggcccaccagcctttttgggntcccccaanaacccgtaaaagccggttntncanncaan

SEQ ID NO: 9 - DNA, 1773 bp, Homo sapiens
ggcacgagcttccgagtaggatgtcactgagatccctcaaatggagcctcctgctgctgtcactcctgagtttctttgtgatgtggtacctcagccttccccactacaatgtgatagaacgcgtgaactggatgtacttctatgagtatgagccgatttacagacaagactttcacttcacacttcgagagcattcaaactgctctcatcaaaatccatttctggtcattctggtgacctcccacccttcagatgtgaaagccaggcaggccattagagttacttggggtgaaaaaaagtcttggtggggatatgaggttcttacatttttcttattaggccaagaggctgaaaaggaagacaaaatgttggcattgtccttagaggatgaacaccttctttatggtgacataatccgacaagattttttagacacatataataacctgaccttgaaaaccattatggcattcaggtgggtaactgagttttgccccaatgccaagtacgtaatgaagacagacactgatgttttcatcaatactggcaatttagtgaagtatcttttaaacctaaaccactcagagaagtttttcacaggttatcctctaattgataattattcctatagaggattttaccaaaaaacccatatttcttaccaggagtatcctttcaaggtgttccctccatactgcagtgggttgggttatataatgtccagagatttggtgccaaggatctatgaaatgatgggtcacgtaaaacccatcaagtttgaagatgtttatgtcgggatctgtttgaatttattaaaagtgaacattcatattccagaagacacaaatcttttctttctatatagaatccatttggatgtctgtcaactgagacgtgtgattgcagcccatggcttttcttccaaggagatcatcactttttggcaggtcatgctaaggaacaccacatgccattattaacttcacattctacaaaaagcctagaaggacaggatactttgtggaaagtgttaaataaagtaggtactgtggaaaattcatggggaggtcagtgtgctggcttacactgaactgaaactcatgaaaaacccagactggagactggagggttacacttgtgatttattagtcaggcccttcaaagatgatatgtggaggaattaaatataaaggaattggaggtttttgctaaagaaattaataggaccaaacaatttggacatgtcattctgtagactagaatttcttaaaagggtgttactgagttataagctcactaggctgtaaaaacaaaacaatgtaagttttatttattgaacaatgtagtcacttgaaggttttgtgtatatcttatgtggattaccaatttaaaaatatatgtagttctgtgtcaaaaaacttcttcactgaagttatctgaacaaaattttacctgtttttggtcatttataaagtacttcaagatgttgcagtatttcacagttattattatttaaaattacttcaactttgtgtttttaaatgttttgacgatttcaatacaagataaaaaggatagtgaatcattctttacatgcaaacattttccagttacttaactgatcagtttattattgatacatcactccattaatgtaaagtcataggtcattattgcatatcagtaatctcttggactttgttaaatattttactgtggtaatatagagaagaattaaagcaagaaaatctgaaaaaaaaaaaaaaaaaaaaa

SEQ ID NO: 10 - protein, 320 aa, Homo sapiens
MSLRSLKWSLLLLSLLSFFVMWYLSLPHYNVIERVNWMYFYEYEPIYRQDFHFTLREHSNCSHQNPFLVILVTSHPSDVKARQAIRVTWGEKKSWWGYEVLTFFLLGQEAEKEDKMLALSLEDEHLLYGDIIRQDFLDTYNNLTLKTIMAFRWVTEFCPNAKYVMKTDTDVFINTGNLVKYLLNLNHSEKFFTGYPLIDNYSYRGFYQKTHISYQEYPFKVFPPYCSGLGYIMSRDLVPRIYEMMGHVKPIKFEDVYVGICLNLLKVNIHIPEDTNLFFLYRIHLDVCQLRRVIAAHGFSSKEIITFWQVMLRNTTCHYX

SEQ ID NO: 11 - DNA, 1271 bp, Homo sapiens; identical to SEQ ID NO: 3

SEQ ID NO: 12 - protein, 353 aa, Homo sapiens
MLCRLCWLVSYSLAVLLLGCLLFLRKAAKPAETPRPTSLSGAPPTPRHSRCPPNHTVSSASLSLPSRHRLFLTYRHCRNFSILLEPSGCSKDTFLLLAIKSQPGHVERRAAIRSTWGRWGDGLGPALKLVFLLGVAGSAPPAQLLAYESREFDDILQWDFTEDFFNLTLKELHLQRWVVAACPQAHFMLKGDDDVFVHVPNVLEFLDGWDPAQDLLVGDVIRQALPNRNTKVKYFIPPSMYRATHYPPYAGGGGYVMSRATVRRLQAIMEDAELFPIDDVFVGMCLRRLGLSPMHHAGFKTFGIRRPLDPLDPCLYRGLLLVHRLSPLEMWTMWALVTDEGLKCAAGPIPQRX

SEQ ID NO: 13 - DNA, 29 bp, Homo sapiens
caattggatcccttccccactacaatgtg

SEQ ID NO: 14 - DNA, 29 bp, Homo sapiens
gtacgcaagataatggcatgtggtgttcc

SEQ ID NO: 15 - DNA, 31 bp, Homo sapiens
cgcggatccgccatcatgtcactgagatccc

SEQ ID NO: 16 - DNA, 32 bp, Homo sapiens
cacttaggtaccataatggcatgtggtgttcc

(19) United States
(12) Patent Application Publication
(10) Pub. No.: US 2001/0024813 A1
(43) Pub. Date: Sep. 27, 2001

(54) Human dendriac and brainiac-3

(76) Inventors: Reinhard Ebner, Gaithersburg, MD (US); Daniel R. Soppet, Centreville, VA (US); Gregory A. Endress, Potomac, MD (US); Kimberly A. Florence, Rockville, MD (US); Guo-Liang Yu, Berkeley, CA (US); Steven M. Ruben, Olney, MD (US); Craig A. Rosen, Laytonsville, MD (US)

Correspondence Address: HUMAN GENOME SCIENCES INC, 9410 KEY WEST AVENUE, ROCKVILLE, MD, 20850

(21) Appl. No.: 09/739,451

(22) Filed: Dec. 19, 2000

Related U.S. Application Data
(63) Continuation of application No. 09/213,364, filed on Dec. 17, 1998, now abandoned.
(60) Provisional application No. 60/108,928, filed on Nov. 17, 1998.
(60) Provisional application No. 60/077,687, filed on Mar. 12, 1998.
(60) Provisional application No. 60/068,006, filed on Dec. 18, 1997.

(51) Int. Cl. C12N 9/00; C07H 21/04; C12P 21/02; C12N 5/06

(52) U.S. Cl. 435/183; 435/69.1; 435/325; 536/23.2

(57) ABSTRACT

The present invention relates to novel Dendriac and novel Brainiac-3 polypeptides which are members of the Brainiac family. In particular, isolated nucleic acid molecules are provided encoding the human Dendriac and Brainiac-3 polypeptides. Dendriac and Brainiac-3 polypeptides are also provided as are vectors, host cells and recombinant methods for producing the same. The invention further relates to screening methods for identifying agonists and antagonists of Dendriac and Brainiac-3 activities. Also provided are diagnostic methods for detecting immune and nervous system-related disorders and therapeutic methods for treating immune and nervous system-related disorders.

This application claims benefit under 35 U.S.C. § 119(e) of the filing date of the following copending U.S. Provisional Applications: Ser. No. 60/108,928, filed on Nov. 17, 1998; Ser. No. 60/077,687, filed on Mar. 12, 1998; and Ser. No. 60/068,006, filed on Dec. 18, 1997. Each of the three aforementioned applications are hereby incorporated by reference in their entireties.

### FIELD OF THE INVENTION

The present invention relates to two novel human genes encoding polypeptides related to the Notch family. More specifically, isolated nucleic acid molecules are provided encoding two human polypeptides named Dendriac (also termed Brainiac-2) and Brainiac-3. Dendriac and Brainiac-3 polypeptides are also provided, as are vectors, host cells and recombinant methods for producing the same. Also provided are diagnostic methods for detecting disorders related to the immune and nervous systems, and therapeutic methods for treating such disorders.

The invention further relates to screening methods for identifying agonists and antagonists of Dendriac and/or Brainiac-3 activity.

### BACKGROUND OF THE INVENTION

Control of cell division is a basic aspect of multicellular existence that depends upon a programmed series of events. One factor in cellular proliferation and its control is the presence of various polypeptide growth factors. Growth factors are essential components of growth media for in vitro cell culture and are involved in cell survival in vivo. A partial list of growth factors identified to date include platelet-derived growth factor (PDGF; implicated in the repair of the vascular system in vivo); epidermal growth factor (EGF; which acts as a mitogen for cells of ectodermal and mesodermal origin); transforming growth factor (TGF)-α (which acts as a mitogen similarly to EGF, with the exception that it enables normal cells to grow in soft-agar); transforming growth factor (TGF)-β (a mitogen for some cells and a growth inhibitor for others); and nerve growth factor (NGF; which is involved in the development and maintenance of sympathetic and embryonic neurons). (Watson, et al., Molecular Biology of the Gene, p. 975; Benjamin/Cummings (1987).)

It is clear that particular cell types require particular growth factors for normal growth and maintenance. Peptide growth factors are produced and secreted from a variety of tissues. The target cells are typically located near the site of release of the growth factor (paracrine response). In addition to growth promoting and differentiation-inducing activities, growth factors elicit a wide variety of effects on their target cells and are involved in processes such as inflammation, immune reactions, and wound repair. (See, Pimentel, E. Handbook of Growth Factors, Volume 1: General Basics (CRC Press 1994).)

Myocardial hypertrophy refers to a focal or general enlargement of the heart. Normal hypertrophy is a compensatory action which functions to maintain the pumping action of the heart. Abnormal hypertrophy occurs in a number of situations including hypertension, myocardial infarction, valve disease, and cardiomyopathy. (Simpson, P. C. HeartFailure 5:113 (1989).) The effects of peptide growth factors on cardiac myocytes are reflected in differentiated patterns of gene expression. For example, stimulation of the α-adrenergic receptor induces hypertrophy of cultured cardiac myocytes and produces specific changes in gene expression at the level of transcription. (Simpson, P. C. “Cardiac Myocyte Hypertrophy, ” Molecular Biology of the Cardiovascular System, Roberts, R. et al., ed.: 125-133 (1990).) In cardiac myocytes, the growth factors TGF-β1 and basic FGF concomitantly elicit complex and heterogeneous responses: selective inhibition of certain adult transcripts, concurrent with the upregulation of “fetal” contractile protein genes. (Schneider, et al., “Oncogenes and Myogenesis, ” Molecular Biology of the Cardiovascular System, Roberts, R. et al., ed.: 63-71 (1990).)

Monitoring of growth factor gene expression in myocytes and other cells of the heart, including connective tissue, would be useful in detecting and studying abnormal hypertrophy both in vitro and in vivo. Organ and clonal cell systems have been developed to analyze cardiomyogenic differentiation. (See, for example, Bader, D. et al., Molecular Biology of the Cardiovascular System, Roberts, R. et al., ed.: 41-49 (1990).) Differentiation in these systems can be monitored by in vitro analysis of cardiac myogenesis and monoclonal antibodies that have been raised against muscle-specific protein.

Additionally, polypeptide growth factors are very important cell culture reagents for stimulating cellular growth and aiding survival of the cells in vitro.

The search continues to exist for polypeptides that stimulate and/or inhibit growth of particular cells for both in vitro and in vivo uses. In addition, the search continues for novel tissue specific markers that can be employed qualitatively to help identify a particular cell or tissue type and employed qualitatively to assess whether cells, tissues or organs are abnormal in their expression of a particular polypeptide.

### SUMMARY OF THE INVENTION

The present invention provides isolated nucleic acid molecules comprising a polynucleotide encoding at least a portion of the Dendriac polypeptide having the complete amino acid sequence shown in SEQ ID NO:2 or the complete amino acid sequence encoded by the cDNA clone deposited as plasmid DNA as ATCC Deposit Number 203056 on Jul. 9, 1998. The nucleotide sequence determined by sequencing the deposited Dendriac clone, which is shown in FIGS. 1A, 1B, and 1C (SEQ ID NO:1), contains an open reading frame encoding a complete polypeptide of 319 amino acid residues, including an initiation codon encoding an N-terminal methionine at nucleotide positions 426-428, and a predicted molecular weight of about 38,197 Daltons. Nucleic acid molecules of the invention include those encoding the complete amino acid sequence excepting the N-terminal methionine shown in SEQ ID NO:2, or the complete amino acid sequence excepting the N-terminal methionine encoded by the cDNA clone in ATCC Deposit Number 203056, which molecules also can encode additional amino acids fused to the N-terminus of the Dendriac amino acid sequence.

The present invention provides isolated nucleic acid molecules comprising a polynucleotide encoding at least a portion of the Dendriac polypeptide having the complete amino acid sequence shown in SEQ ID NO:10 or the complete amino acid sequence encoded by the cDNA clone deposited as plasmid DNA in a pool of 50 distinct plasmid DNA molecules as ATCC Deposit Number 209627 on Feb. 12, 1998. The nucleotide sequence determined by sequencing the deposited Dendriac clone, which is shown as SEQ ID NO:9, contains an open reading frame encoding a complete polypeptide of 319 amino acid residues, including an initiation codon encoding an N-terminal methionine at nucleotide positions 21-23, and a predicted molecular weight of about 38,197 Daltons. Nucleic acid molecules of the invention include those encoding the complete amino acid sequence excepting the N-terminal methionine shown in SEQ ID NO: 10, or the complete amino acid sequence excepting the N-terminal methionine encoded by the cDNA clone in ATCC Deposit Number 209627, which molecules also can encode additional amino acids fused to the N-terminus of the Dendriac amino acid sequence.

The present invention also provides isolated nucleic acid molecules comprising a polynucleotide encoding at least a portion of the Brainiac-3 polypeptide having the complete amino acid sequence shown in SEQ ID NO:4 or the complete amino acid sequence encoded by the cDNA clone deposited as plasmid DNA as ATCC Deposit Number 203451 on Nov. 9, 1998. The nucleotide sequence determined by sequencing the deposited Brainiac-3 clone, which is shown in FIGS. 2A and 2B (SEQ ID NO:3), contains an open reading frame encoding a complete polypeptide of 352 amino acid residues, including an initiation codon encoding an N-terminal methionine at nucleotide positions 47-49, and a predicted molecular weight of about 39,521 Daltons. Nucleic acid molecules of the invention include those encoding the complete amino acid sequence excepting the N-terminal methionine shown in SEQ ID NO:4, or the complete amino acid sequence excepting the N-terminal methionine encoded by the cDNA clone in ATCC Deposit Number 203451, which molecules also can encode additional amino acids fused to the N-terminus of the Brainiac-3 amino acid sequence.

The present invention also provides isolated nucleic acid molecules comprising a polynucleotide encoding at least a portion of the Brainiac-3 polypeptide having the complete amino acid sequence shown in SEQ ID NO: 12 or the complete amino acid sequence encoded by the cDNA clone deposited as plasmid DNA in a pool of 50 distinct plasmid DNA molecules encoding 50 distinct molecules as ATCC Deposit Number 209463 on Nov. 14, 1997. The nucleotide sequence determined by sequencing the deposited Brainiac-3 clone, which is shown in SEQ ID NO:11, contains an open reading frame encoding a complete polypeptide of 352 amino acid residues, including an initiation codon encoding an N-terminal methionine at nucleotide positions 47-49, and a predicted molecular weight of about 39,521 Daltons. Nucleic acid molecules of the invention include those encoding the complete amino acid sequence excepting the N-terminal methionine shown in SEQ ID NO:12, or the complete amino acid sequence excepting the N-terminal methionine encoded by the cDNA clone in ATCC Deposit Number 209463, which molecules also can encode additional amino acids fused to the N-terminus of the Brainiac-3 amino acid sequence.

The encoded Dendriac polypeptide has a predicted leader sequence of 25 amino acids underlined in FIGS. 1A, 1B, and 1C; and the amino acid sequence of the predicted mature Dendriac polypeptide is also shown in FIGS. 1A, 1B, and 1C, and in SEQ ID NO:2 and SEQ ID NO:10, as amino acid residues 26-319.

The encoded Brainiac-3 polypeptide has a predicted leader sequence of 28 amino acids underlined in FIGS. 2A and 2B; and the amino acid sequence of the predicted mature Brainiac-3 polypeptide is also shown in FIGS. 2A and 2B, and in SEQ ID NO:4 and SEQ ID NO:12, as amino acid residues 29-352.

Thus, one embodiment of the invention provides an isolated nucleic acid molecule comprising a polynucleotide having a nucleotide sequence selected from the group consisting of: (a) a nucleotide sequence encoding the Dendriac polypeptide having the complete amino acid sequence in SEQ ID NO:2 and SEQ ID NO:10 (i.e., positions −25 to 294 of SEQ ID NO:2 and SEQ ID NO:10); (b) a nucleotide sequence encoding the Dendriac polypeptide having the complete amino acid sequence in SEQ ID NO:2 and SEQ ID NO: 10 excepting the N-terminal methionine (i.e., positions −24-294 of SEQ ID NO:2 and SEQ ID NO: 10); (c) a nucleotide sequence encoding the predicted mature Dendriac polypeptide having the amino acid sequence at positions 1-294 in SEQ ID NO:2 and SEQ ID NO:10; (d) a nucleotide sequence encoding the Dendriac polypeptide having the complete amino acid sequence encoded by the cDNA clone contained in ATCC Deposit No. 203056 and in ATCC Deposit No. 209627; (e) a nucleotide sequence encoding the Dendriac polypeptide having the complete amino acid sequence excepting the N-terminal methionine encoded by the cDNA clone contained in ATCC Deposit No. 203056 and in ATCC Deposit No. 209056; (f) a nucleotide sequence encoding the mature Dendriac polypeptide having the amino acid sequence encoded by the cDNA clone contained in ATCC Deposit No. 203056 and in ATCC Deposit No. 209056; and (g) a nucleotide sequence complementary to any of the nucleotide sequences in (a), (b), (c), (d), (e) or (f), above.

A further embodiment of the invention provides an isolated nucleic acid molecule comprising a polynucleotide having a nucleotide sequence selected from the group consisting of: (a) a nucleotide sequence encoding the Brainiac-3 polypeptide having the complete amino acid sequence in SEQ ID NO:4 and SEQ ID NO:12 (i.e., positions −28 to 324 of SEQ ID NO:4); (b) a nucleotide sequence encoding the Brainiac-3 polypeptide having the complete amino acid sequence in SEQ ID NO:4 and SEQ ID NO:12 excepting the N-terminal methionine (i.e., positions −27-324 of SEQ ID NO:4 and SEQ ID NO:12); (c) a nucleotide sequence encoding the predicted mature Brainiac-3 polypeptide having the amino acid sequence at positions 1-324 in SEQ ID NO:4 and SEQ ID NO:12; (d) a nucleotide sequence encoding the Brainiac-3 polypeptide having the complete amino acid sequence encoded by the cDNA clone contained in ATCC Deposit No. 203451 and in ATCC Deposit No. 209463; (e) a nucleotide sequence encoding the Brainiac-3 polypeptide having the complete amino acid sequence excepting the N-terminal methionine encoded by the cDNA clone contained in ATCC Deposit No. 203451 and in ATCC Deposit No. 209463; (f) a nucleotide sequence encoding the mature Brainiac-3 polypeptide having the amino acid sequence encoded by the cDNA clone contained in ATCC Deposit No. 203451 and in ATCC Deposit No. 209463; and (g) a nucleotide sequence complementary to any of the nucleotide sequences in (a), (b), (c), (d), (e) or (f), above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e), (f) or (g), with regard to Dendriac and Brainiac-3, above, or a polynucleotide which hybridizes under stringent hybridization conditions to a polynucleotide in (a), (b), (c), (d), (e), (f) or (g), with regard to Dendriac and Brainiac-3, above. This polynucleotide which hybridizes does not hybridize under stringent hybridization conditions to a polynucleotide having a nucleotide sequence consisting of only A residues or of only T residues.

An additional nucleic acid embodiment of the invention relates to an isolated nucleic acid molecule comprising a polynucleotide which encodes the amino acid sequence of an epitope-bearing portion of a Dendriac and Brainiac-3 polypeptide having an amino acid sequence in (a), (b), (c), (d), (e) or (f), with regard to Dendriac and Brainiac-3, above. A further nucleic acid embodiment of the invention relates to an isolated nucleic acid molecule comprising a polynucleotide which encodes the amino acid sequence of a Dendriac and Brainiac-3 polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a polynucleotide which encodes the amino acid sequence of a Dendriac or Brainiac-3 polypeptide to have an amino acid sequence which contains not more than 10, 9, 8, 7, 6, 5, 4, 3, 2 or 1 conservative amino acid substitutions.

The present invention also relates to recombinant vectors, which include the isolated nucleic acid molecules of the present invention, and to host cells containing the recombinant vectors, as well as to methods of making such vectors and host cells and for using them for production of Dendriac or Brainiac-3 polypeptides or peptides by recombinant techniques.

The invention also provides an isolated Dendriac polypeptide comprising an amino acid sequence selected from the group consisting of: (a) the amino acid sequence of the full-length Dendriac polypeptide having the complete amino acid sequence shown in SEQ ID NO:2 and SEQ ID NO:10 (i.e., positions 1-319 of SEQ ID NO:2 and SEQ ID NO:10); (b) the amino acid sequence of the full-length Dendriac polypeptide having the complete amino acid sequence shown in SEQ ID NO:2 and SEQ ID NO:10 excepting the N-terminal methionine (i.e., positions 2-319 of SEQ ID NO:2 and SEQ ID NO:10); (c) the amino acid sequence of the predicted mature Dendriac polypeptide having the amino acid sequence at positions 26-319 in SEQ ID NO:2 and SEQ ID NO: 10; (d) the complete amino acid sequence encoded by the cDNA clone contained in the ATCC Deposit No. 203056 and in ATCC Deposit No. 209627; (e) the complete amino acid sequence excepting the N-terminal methionine encoded by the cDNA clone contained in the ATCC Deposit No. 203056 and in ATCC Deposit No. 209627; and (f) the complete amino acid sequence of the predicted mature Dendriac polypeptide encoded by the cDNA clone contained in the ATCC Deposit No. 203056 and in ATCC Deposit No. 209627.

In addition, the invention provides an isolated Brainiac-3 polypeptide comprising an amino acid sequence selected from the group consisting of: (a) the amino acid sequence of the full-length Brainiac-3 polypeptide having the complete amino acid sequence shown in SEQ ID NO:4 and SEQ ID NO:12 (i.e., positions 1-352 of SEQ ID NO:4 and SEQ ID NO:12); (b) the amino acid sequence of the full-length Brainiac-3 polypeptide having the complete amino acid sequence shown in SEQ ID NO:4 and SEQ ID NO:12 excepting the N-terminal methionine (i.e., positions 2-352 of SEQ ID NO:4 and SEQ ID NO: 12); (c) the amino acid sequence of the predicted mature Brainiac-3 polypeptide having the amino acid sequence at positions 29-352 in SEQ ID NO:4 and SEQ ID NO:12; (d) the complete amino acid sequence encoded by the cDNA clone contained in the ATCC Deposit No. 203451 and in ATCC Deposit No. 209463; (e) the complete amino acid sequence excepting the N-terminal methionine encoded by the cDNA clone contained in the ATCC Deposit No. 203451 and in ATCC Deposit No. 209463; and (f) the complete amino acid sequence of the predicted mature Brainiac-3 polypeptide encoded by the cDNA clone contained in the ATCC Deposit No. 203451 and in ATCC Deposit No. 209463.

The polypeptides of the present invention also include polypeptides having an amino acid sequence at least 80% identical, more preferably at least 90% identical, and still more preferably 95%, 96%, 97%, 98% or 99% identical to those described in (a), (b), (c), (d), (e) or (f), of Dendriac and Brainiac-3, above, as well as polypeptides having an amino acid sequence with at least 90% similarity, and more preferably at least 95% similarity, to Dendriac and Brainiac-3, above.

An additional embodiment of the invention relates to a peptide or polypeptide which comprises the amino acid sequence of an epitope-bearing portion of a Dendriac and/or Brainiac-3, polypeptide having an amino acid sequence described in (a), (b), (c), (d), (e) or (f), with regard to Dendriac and Brainiac-3, above. Peptides or polypeptides having the amino acid sequence of an epitope-bearing portion of a Dendriac and/or Brainiac-3 polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

A further embodiment of the invention relates to a peptide or polypeptide which comprises the amino acid sequence of a Dendriac and/or Brainiac-3 polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a peptide or polypeptide to have an amino acid sequence which comprises the amino acid sequence of a Dendriac and/or Brainiac-3 polypeptide, which contains at least one, but not more than 10, 9, 8, 7, 6, 5, 4, 3, 2 or 1 conservative amino acid substitutions.

In another embodiment, the invention provides an isolated antibody that binds specifically to a Dendriac and Brainiac-3 polypeptide having an amino acid sequence described in (a), (b), (c), (d), (e) or (f), with regard to Dendriac and Brainiac-3, above. The invention further provides methods for isolating antibodies that bind specifically to a Dendriac and/or Brainiac-3 polypeptide having an amino acid sequence as described herein. Such antibodies are useful diagnostically or therapeutically as described below.

The invention also provides for pharmaceutical compositions comprising Dendriac and/or Brainiac-3 polypeptides, particularly human Dendriac and/or Brainiac-3 polypeptides, which may be employed, for instance, to treat immune and/or nervous system diseases and disorders. Methods of treating individuals in need of Dendriac and/or Brainiac-3 polypeptides are also provided.

The invention further provides compositions comprising a Dendriac and/or Brainiac-3 polynucleotide or a Dendriac and/or Brainiac-3 polypeptide for administration to cells in vitro, to cells ex vivo and to cells in vivo, or to a multicellular organism. In certain particularly preferred embodiments of this aspect of the invention, the compositions comprise a Dendriac and/or Brainiac-3 polynucleotide for expression of a Dendriac and/or Brainiac-3 polypeptide in a host organism for treatment of disease. Particularly preferred in this regard is expression in a human patient for treatment of a dysfunction associated with aberrant endogenous activity of a Dendriac and/or Brainiac-3 polynucleotide and/or polypeptide.

The present invention also provides a screening method for identifying compounds capable of enhancing or inhibiting a biological activity of the Dendriac and/or Brainiac-3 polypeptide, which involves contacting a receptor whose activity is inhibited or enhanced by the Dendriac and/or Brainiac-3 polypeptide with the candidate compound in the presence of a Dendriac and/or Brainiac-3 polypeptide, assaying cell division activity of the receptor in the presence of the candidate compound and of Dendriac and/or Brainiac-3 polypeptide, and comparing the receptor activity to a standard level of activity, the standard being assayed when contact is made between the receptor and in the presence of the Dendriac and/or Brainiac-3 polypeptide and the absence of the candidate compound In this assay, an increase in receptor activity over the standard indicates that the candidate compound is an agonist of Dendriac and/or Brainiac-3 activity and a decrease in receptor activity compared to the standard indicates that the compound is an antagonist of Dendriac and/or Brainiac-3 activity.

In another aspect, a screening assay for agonists and antagonists is provided which involves determining the effect a candidate compound has on Dendriac and/or Brainiac-3 binding to another member of the Notch family. In particular, the method involves contacting the Notch family member with a Dendriac and/or Brainiac-3 polypeptide and a candidate compound and determining whether Dendriac and/or Brainiac-3 polypeptide binding to the Notch family member is increased or decreased due to the presence of the candidate compound. In this assay, an increase in binding of Dendriac and/or Brainiac-3 over the standard binding indicates that the candidate compound is an agonist of Dendriac and/or Brainiac-3 binding activity and a decrease in Dendriac and/or Brainiac-3 binding compared to the standard indicates that the compound is an antagonist of Dendriac and/or Brainiac-3 binding activity.

In yet another aspect, the Dendriac and/or Brainiac-3 polypeptide(s) may bind to a cell surface polypeptide which also function as a viral receptor or coreceptor. Thus, Dendriac and/or Brainiac-3, or agonists or antagonists thereof, may be used to regulate viral infectivity at the level of viral binding or interaction with the Dendriac and/or Brainiac-3 receptor or coreceptor or during the process of viral internalization or entry into the cell.

It has been discovered that Dendriac is expressed not only in dendritic cells, but also (using BLAST analysis of the HGS EST database) in NTERA2 cells, adult pulmonary tissue, salivary gland, ovary, Caco-2 colon adenocarcinoma cell line, smooth muscle, cerebellum, 8 week old whole human embryo, hemagiopericytoma, amygdala, substantia nigra, and whole brain. Further, (using Northern blot analysis) the Dendriac message is abundantly detected in brain, kidney, pancreas, testis, fetal liver, and thyroid. In addition, Northern blot experiments show lower, but clear, levels of expression of Dendriac in the following tissues: lung, liver, amygdala, caudate nucleus, corpus callosum, hippocampus, whole brain, substantia nigra, subthalamic nucleus, thalamus, adrenal cortex, small intestine, stomach, spleen, lymph node, and colorectal adenocarcinoma SW480. Finally, very low levels of Dendriac expression are observed by Northern blot in the following tissues: prostate, uterus, lung carcinoma A549, and melanoma G361. Therefore, nucleic acids of the invention are useful as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cells, particularly of the immune system, significantly higher or lower levels of Dendriac gene expression may be detected in certain tissues (e.g., cancerous and wounded tissues) or bodily fluids (e.g., serum, plasma, urine, synovial fluid or spinal fluid) taken from an individual having such a disorder, relative to a standard Dendriac gene expression level, i.e., the Dendriac expression level in healthy tissue from an individual not having the immune system disorder.

Thus, the invention provides a diagnostic method useful during diagnosis of such a disorder, which involves: (a) assaying Dendriac gene expression level in cells or body fluid of an individual; (b) comparing the Dendriac gene expression level with a standard Dendriac gene expression level, whereby an increase or decrease in the assayed Dendriac gene expression level compared to the standard expression level is indicative of disorder in the immune system.

It has been discovered that Brainiac-3 is expressed not only in fetal brain, but also in epileptic frontal cortex, and 12 week old early stage human. Further, (using Northern blot analysis) the Brainiac-3 message is abundantly detected in fetal brain and fetal kidney. In addition, Northern blot experiments also show lower, but clear, levels of expression of Brainiac-3 in the lung and liver. Further, the Brainiac-3 Northern blot expression studies identify an approximately 1.35 kb band in all positive tissues, an approximately 2.0 kb band in fetal kidney and fetal brain, and an approximately 4.0 kb band in fetal brain. Therefore, nucleic acids of the invention are useful as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cells, particularly of the immune and nervous systems, significantly higher or lower levels of Brainiac-3 gene expression may be detected in certain tissues (e.g., cancerous and wounded tissues) or bodily fluids (e.g., serum, plasma, urine, synovial fluid or spinal fluid) taken from an individual having such a disorder, relative to a standard Brainiac-3 gene expression level, i.e., the Brainiac-3 expression level in healthy tissue from an individual not having the immune system disorder.

Thus, the invention provides a diagnostic method useful during diagnosis of such a disorder, which involves: (a) assaying Brainiac-3 gene expression level in cells or body fluid of an individual; (b) comparing the Brainiac-3 gene expression level with a standard Brainiac-3 gene expression level, whereby an increase or decrease in the assayed Brainiac-3 gene expression level compared to the standard expression level is indicative of disorder in the immune and nervous systems.

Another embodiment of the invention is related to a method for treating an individual in need of an increased level of Dendriac activity in the body comprising administering to such an individual a composition comprising a therapeutically effective amount of an isolated Dendriac polypeptide of the invention or an agonist thereof.

A further embodiment of the invention is related to a method for treating an individual in need of a decreased level of Dendriac activity in the body comprising, administering to such an individual a composition comprising a therapeutically effective amount of a Dendriac antagonist. Preferred antagonists for use in the present invention are Dendriac-specific antibodies.

An still further embodiment of the invention is related to a method for treating an individual in need of an increased level of Brainiac-3 activity in the body comprising administering to such an individual a composition comprising a therapeutically effective amount of an isolated Brainiac-3 polypeptide of the invention or an agonist thereof.

An even further embodiment of the invention is related to a method for treating an individual in need of a decreased level of Brainiac-3 activity in the body comprising, administering to such an individual a composition comprising a therapeutically effective amount of a Brainiac-3 antagonist. Preferred antagonists for use in the present invention are Brainiac-3-specific antibodies.

### BRIEF DESCRIPTION OF THE FIGURES

FIGS. 1A, 1B, and 1C show the nucleotide sequence (SEQ ID NO:1) and deduced amino acid sequence (SEQ ID NO:2) of Dendriac. The predicted leader sequence of about 25 amino acids is underlined. Five potential 25 asparagine-linked glycosylation sites are marked in the amino acid sequence of Dendriac. The potential sites of glycosylation begin at asparagine-60, asparagine-142, asparagine-186, asparagine-200, and asparagine-314 in FIGS. 1A, 1B, and 1C (these positions correspond to the identical sequence located at asparagine-35, asparagine-117, asparagine-160, asparagine-175, and asparagine-289 in SEQ ID NO:2 and in SEQ ID NO: 10). The potential glycosylation sites are marked with a bold pound symbol (#) above the nucleotide sequence coupled with a bolded one letter abbreviation for the asparagine (N) in the amino acid sequence in FIGS. 1A, 1B, and 1C.

Regions of high identity between Dendriac and Brainiac-3 and the closely related Drosophila Brainiac (an alignment of these sequences is presented in FIG. 3) are delineated in FIGS. 1A, 1B, and 1C with a double underline. These regions are not limiting and are labeled as Conserved Domain (CD)-I, CD-II, CD-III, CD-IV, CD-V, CD-VI, CD-VII, CD-VIII, CD-IX, CD-X, and CD-XI in FIGS. 1A, 1B, and 1C.

FIGS. 2A and 2B show the nucleotide sequence (SEQ ID NO:3) and deduced amino acid sequence (SEQ ID NO:4) of Brainiac-3. The predicted leader sequence of about 28 amino acids is underlined. Three potential asparagine-linked glycosylation sites are marked in the amino acid sequence of Brainiac-3. The potential sites of glycosylation begin at asparagine-54, asparagine-79, and asparagine-166 in FIGS. 2A and 2B (these positions correspond to the identical sequence located at asparagine-26, asparagine-51, and asparagine-138 in SEQ ID NO:4 and in SEQ ID NO:12). The potential glycosylation sites are marked with a bold pound symbol (#) above the nucleotide sequence coupled with a bolded one letter abbreviation for the asparagine (N) in the amino acid sequence in FIGS. 2A and 2B.

Regions of high identity between Dendriac and Brainiac-3 and the closely related Drosophila Brainiac (an alignment of these sequences is presented in FIG. 3) are delineated in FIGS. 2A and 2B with a double underline. These regions are not limiting and are labeled as Conserved Domain (CD)-I, CD-II, CD-III, CD-IV, CD-V, CD-VI, CD-VII, CD-VIII, CD-IX, CD-X, and CD-XI. in FIGS. 2A and 2B.

FIG. 3 shows the regions of identity between the amino acid sequences of the Dendriac and Brainiac-3 polypeptides and translation product of the Drosophila melanogaster mRNA for Brainiac (SEQ ID NO:5;

GenBank Accession No. U41449), determined by the computer program MegAlign (DNA*STAR nucleotide and amino acid sequence analysis package) using the default parameters.

FIG. 4 shows an analysis of the Dendriac amino acid sequence. Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the Dendriac polypeptide, i.e., regions from which epitope-bearing peptides of the invention can be obtained.

FIG. 5 shows an analysis of the Brainiac-3 amino acid sequence. Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the Brainiac-3 polypeptide, i.e., regions from which epitope-bearing peptides of the invention can be obtained.

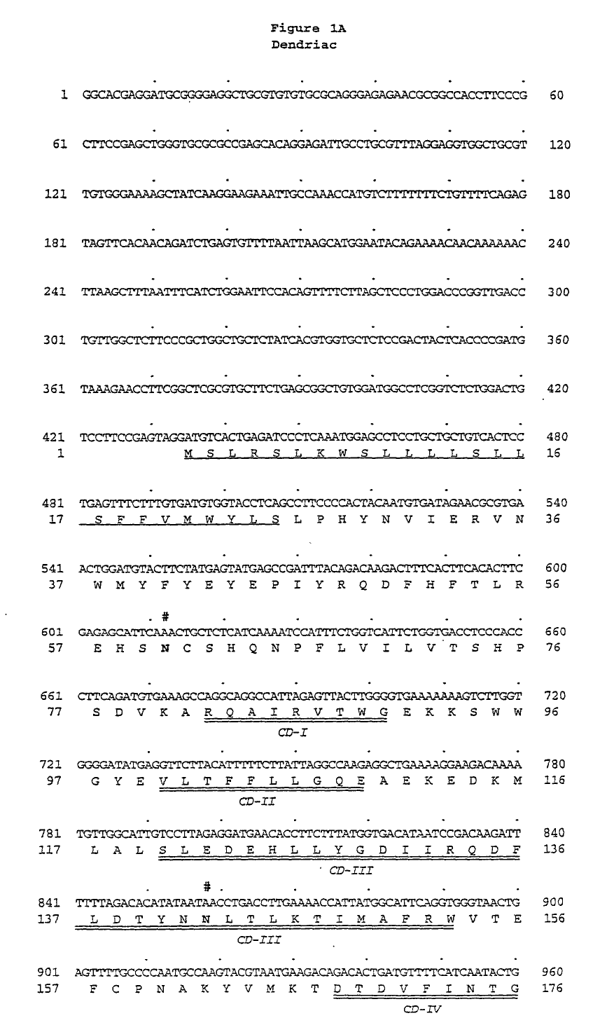

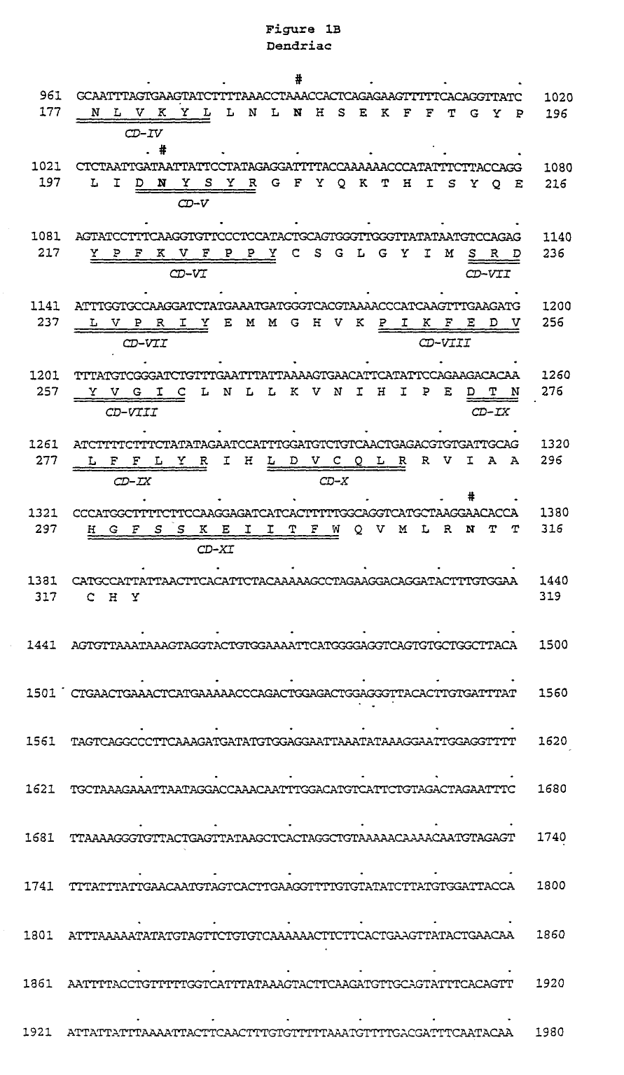

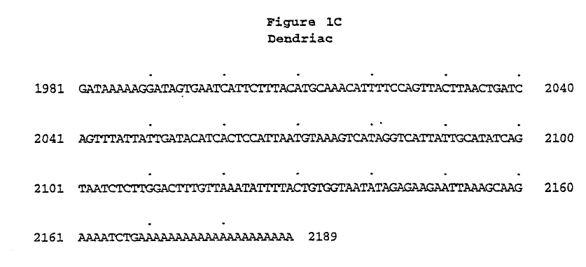

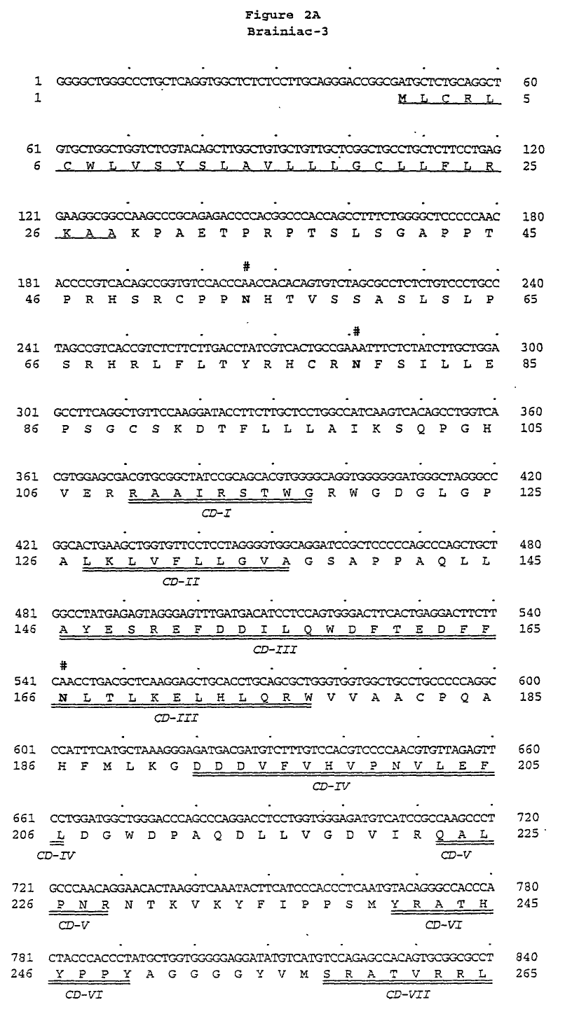

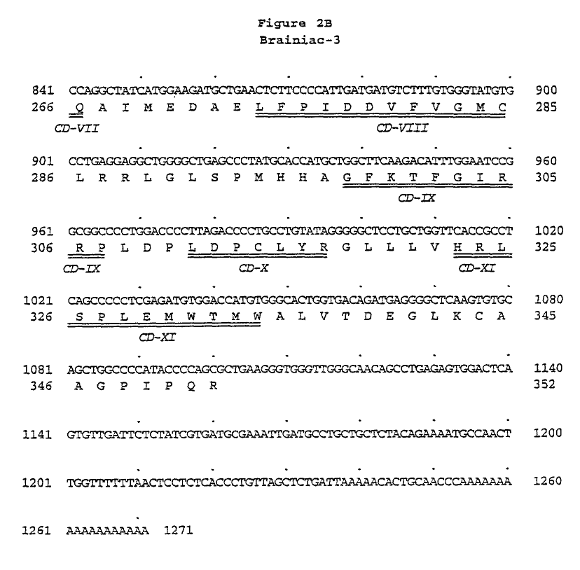

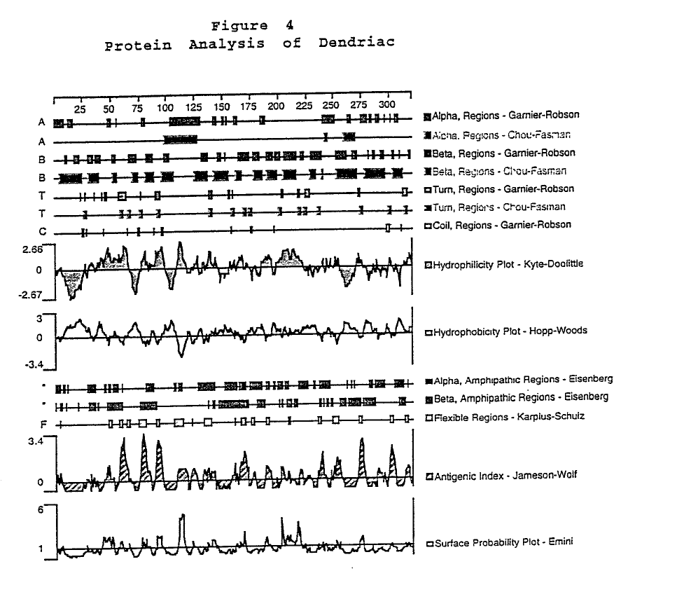

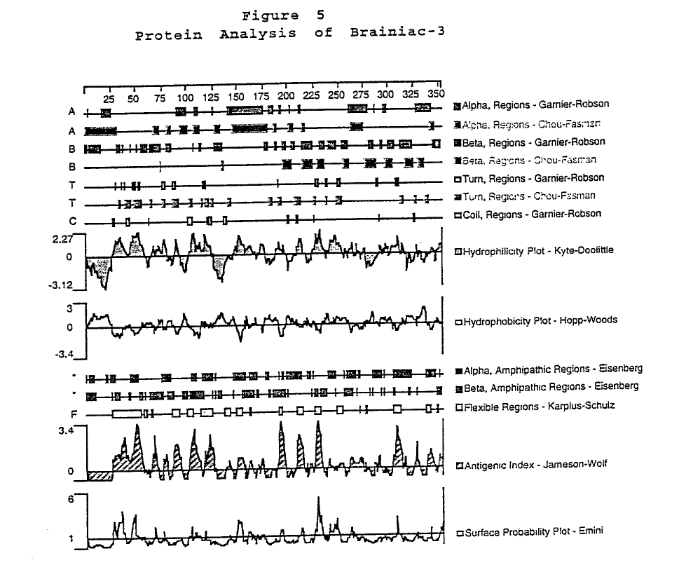

The data presented in FIGS. 4 and 5 are also represented in tabular form in Tables I and II, respectively. The columns in each Table are labeled with the headings “Res”, “Position”, and Roman Numerals I-XIV. The column headings refer to the following features of the amino acid sequence presented in FIGS. 4 and 5, and Tables I and II, respectively: “Res”: amino acid residue of SEQ ID NOs:2 and 4, or FIGS. 1A, 1B, and 1C, and 2A and 2B, respectively; Position: position of the corresponding residue within SEQ ID NOs:2 and 4, or FIGS. 1A, 1B, and 1C, and 2A and 2B, respectively; I: Alpha, Regions-Garnier-Robson; II: Alpha, Regions-Chou-Fasman; III: Beta, Regions-Garnier-Robson; IV: Beta, Regions-Chou-Fasman; V: Turn, Regions-Garnier-Robson; VI: Turn, Regions-Chou-Fasman; VII: Coil, Regions-Garnier-Robson; VIII: Hydrophilicity Plot-Kyte-Doolittle; IX: Hydrophobicity Plot-Hopp-Woods; X: Alpha, Amphipathic Regions-Eisenberg; XI: Beta, Amphipathic Regions-Eisenberg; XII: Flexible Regions-Karplus-Schulz; XIII: Antigenic Index-Jaraeson-Wolf; and XIV: Surface Probability Plot-Emini.

### DETAILED DESCRIPTION

The present invention provides isolated nucleic acid molecules comprising a polynucleotide encoding a Dendriac polypeptide having the amino acid sequence shown in SEQ ID NO:2, which was determined by sequencing a cloned cDNA. The nucleotide sequence shown in FIGS. 1A, 1B, and 1C (SEQ ID NO:1) was obtained by sequencing the HFVIF40 cDNA clone, which was deposited on Jul. 9, 1998 at the American Type Culture Collection, 10801 University Boulevard, Manassas, Va. 20110-2209, and given ATCC accession number 203056. The deposited clone is contained in the pBluescript SK(−) plasmid (Stratagene, La Jolla, Calif.). The nucleotide and amino acid sequences of this clone were presented in U.S. Provisional Application Ser. No. 60/077,687 and are shown in this application as SEQ ID NO:9 and SEQ ID NO:10, respectively.

The determined nucleotide sequence for the MRNA encoding Dendriac of the invention has been translated to provide a determined amino acid sequence shown as SEQ ID NO:10. The determined amino acid sequence for Dendriac shown as SEQ ID NO:10 encoded by the determined nucleotide sequence shown as SEQ ID NO:9, beginning at or near the translation initiation (“start”) codon of the protein and continuing until the first translation termination (“stop”) codon. Translation of the determined nucleotide sequence shown in SEQ ID NO:9 is continued in the reading frame of that first amino acid codon to the first stop codon in that same open reading frame, i.e., to the position in SEQ ID NO:9 which encodes the amino acid at the position in SEQ ID NO: 10 identified as the last amino acid of the open reading frame.

The present invention further provides isolated nucleic acid molecules comprising a polynucleotide encoding a Brainiac-3 polypeptide having the amino acid sequence shown in SEQ ID NO:4, which was determined by sequencing a cloned cDNA. The nucleotide sequence shown in FIGS. 2A and 2B (SEQ ID NO:3) was obtained by sequencing the HFCCQ50 cDNA clone, which was deposited on Nov. 9, 1998 at the American Type Culture Collection, 10801 University Boulevard, Manassas, Va. 20110-2209, and given ATCC accession number 203451. The deposited clone is contained in the pBluescript SK(−) plasmid (Stratagene, La Jolla, Calif.). The nucleotide and amino acid sequences of this clone were presented in U.S. Provisional Application Ser. No. 60/068,006 and are shown in this application as SEQ ID NO:11 and SEQ ID NO:12, respectively.

The determined nucleotide sequence for the MRNA encoding Brainiac-3 of the invention has been translated to provide a determined amino acid sequence shown as SEQ ID NO:12. The determined amino acid sequence for Brainiac-3 shown as SEQ ID NO:12 encoded by the determined nucleotide sequence shown as SEQ ID NO: 11, beginning at or near the translation initiation (“start”) codon of the protein and continuing until the first translation termination (“stop”) codon. Translation of the determined nucleotide sequence shown in SEQ ID NO: 11 is continued in the reading frame of that first amino acid codon to the first stop codon in that same open reading frame, i.e., to the position in SEQ ID NO: 11 which encodes the amino acid at the position in SEQ ID NO:12 identified as the last amino acid of the open reading frame.

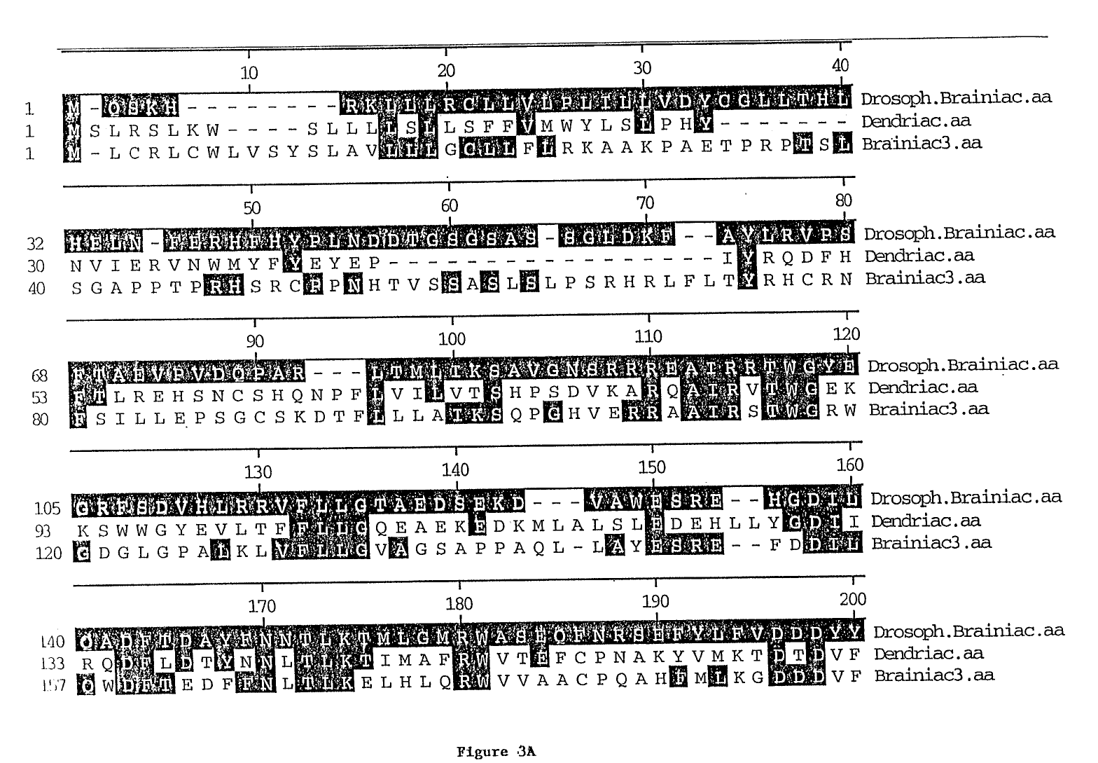

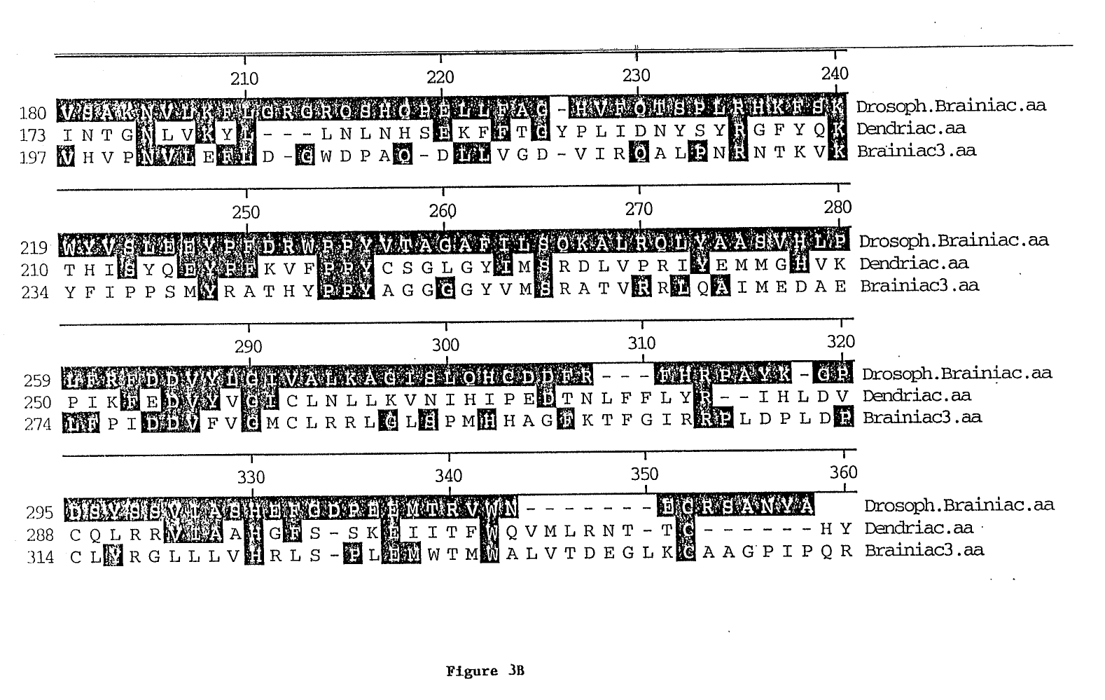

The Dendriac and Brainiac-3 polypeptides of the present invention share sequence homology with the translation product of the Drosophila melanogaster MRNA which encodes Brainiac (FIG. 3; SEQ ID NO:5). Brainiac-3 shares homology with neurogenic secreted signaling protein. Drosophila Brainiac is thought to be an important neurogenic secreted molecule that is believed to play a role in the differentiation of embryonic cells into neurons. Thus, it is contemplated that the Dendriac and Brainiac-3 polynucleotides and polypeptides of the invention exert an effect on the differentiation of cells in the early stages of cell and tissue development, and may serve to aid in the differentiation of embryonic cells into dendritic or other immune system cells or neurons or other cells of the nervous system.

### Nucleic Acid Molecules

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequence in FIGS. 1A, 1B, and 1C (SEQ ID NO:1) and in SEQ ID NO:9, a nucleic acid molecule of the present invention encoding a Dendriac polypeptide may be obtained using standard cloning and screening procedures, such as those for cloning cDNAs using mRNA as starting material. Illustrative of the invention, the nucleic acid molecule described in FIGS. 1A, 1B, and 1C (SEQ ID NO:1) and in SEQ ID NO:9 was discovered in a cDNA library derived from dendritic cells. Additional clones of the same gene were also identified in cDNA libraries from the following tissues: NTERA2 cells, adult pulmonary tissue, salivary gland, ovary, Caco-2 colon adenocarcinoma cell line, smooth muscle, cerebellum, 8 week old whole human embryo, hemagiopericytoma, amygdala, substantia nigra, and whole brain.

Using the information provided herein, such as the nucleotide sequence in FIGS. 2A and 2B (SEQ ID NO:3) and in SEQ ID NO:11, a nucleic acid molecule of the present invention encoding a Brainiac-3 polypeptide may be obtained using standard cloning and screening procedures, such as those for cloning cDNAs using mRNA as starting material. Illustrative of the invention, the nucleic acid molecule described in FIGS. 2A and 2B (SEQ ID NO:3) and in SEQ ID NO: 11 was discovered in a cDNA library derived from fetal brain. Additional clones of the same gene were also identified in cDNA libraries from the following tissues: epileptic frontal cortex, and 12 week old early stage human.

The determined nucleotide sequence of the Dendriac cDNA of FIGS. 1A, 1B, and 1C (SEQ ID NO:1) contains an open reading frame encoding a polypeptide of 319 amino acid residues, with an initiation codon at nucleotide positions 426-428 of the nucleotide sequence in FIGS. 1A, 1B, and 1C (SEQ ID NO:1), and a deduced molecular weight of about 38,197 Daltons. The determined nucleotide sequence of the Dendriac cDNA of SEQ ID NO:9 contains an open reading frame encoding a polypeptide of 319 amino acid residues, with an initiation codon at nucleotide positions 21-23 of the nucleotide sequence in SEQ ID NO:9, and a deduced molecular weight of about 38,197 Daltons. The amino acid sequence of the Dendriac polypeptide shown in SEQ ID NO:2 and SEQ ID NO:10 is about 29.7% identical to Drosophila melanogaster mRNA for Brainiac (FIG. 3), which can be accessed as GenBank Accession No. U41449.

The determined nucleotide sequence of the Brainiac-3 cDNA of FIGS. 2A and 2B (SEQ ID NO:3) and of SEQ ID NO:11 contains an open reading frame encoding a polypeptide of 352 amino acid residues, with an initiation codon at nucleotide positions 47-49 of the nucleotide sequence in FIGS. 2A and 2B (SEQ ID NO:3) and of SEQ ID NO: 11, and a deduced molecular weight of about 39,521 Daltons. The amino acid sequence of the Brainiac-3 polypeptide shown in SEQ ID NO:4 and SEQ ID NO:12 is about 37.3% identical to Drosophila melanogaster mRNA for Brainiac (FIG. 3), which can be accessed as GenBank Accession No. U41449.

As one of ordinary skill would appreciate, due to the possibilities of sequencing errors discussed above, the actual complete Dendriac and Brainiac-3 polypeptides encoded by the respective deposited cDNA clones, which comprises about 319 (Dendriac) and 352 (Brainiac-3) amino acids, may be somewhat longer or shorter. More generally, the actual open reading frames comprising Dendriac and Brainiac-3 may be anywhere in the range of ±20 amino acids, more likely in the range of ±10 amino acids, of that predicted from the methionine codon from the N-terminus shown in FIGS. 1A, 1B, and 1C (SEQ ID NO:1) and SEQ ID NO:9 (i.e., Dendriac) and FIGS. 2A and 2B (SEQ ID NO:3) and SEQ ID NO: 11 (i.e., Brainiac-3). It will further be appreciated that, depending on the analytical criteria used for identifying various functional domains, the exact “address” of the signal sequences of the Dendriac and/or Brainiac-3 polypeptides may differ slightly from the predicted positions above.

Leader and Mature Sequences

The amino acid sequences of the complete Dendriac and Brainiac-3 polypeptides each include a leader sequence and a mature polypeptide, as shown in SEQ ID NO:2 and SEQ ID NO:10 and SEQ ID NO:4 and SEQ ID NO: 12, respectively. More in particular, the present invention provides nucleic acid molecules encoding a mature form of either the Dendriac or Brainiac-3 polypeptide. Thus, according to the signal hypothesis, once export of the growing polypeptide chain across the rough endoplasmic reticulum has been initiated, polypeptides secreted by mammalian cells have a signal or secretory leader sequence which is cleaved from the complete polypeptide to produce a secreted mature form of the polypeptide. Most mammalian cells and even insect cells cleave secreted polypeptides with the same specificity. However, in some cases, cleavage of a secreted polypeptide is not entirely uniform, which results in two or more mature species of the polypeptide. Further, it has long been known that the cleavage specificity of a secreted polypeptide is ultimately determined by the primary structure of the complete polypeptide, that is, it is inherent in the amino acid sequence of the polypeptide. Therefore, the present invention provides a nucleotide sequence encoding the mature Dendriac polypeptide having the amino acid sequence encoded by the cDNA clones contained in ATCC Deposit Nos. 203056 and 209627. The present invention thus also provides a nucleotide sequence encoding the mature Brainiac-3 polypeptide having the amino acid sequence encoded by the cDNA clones contained in ATCC Deposit Nos. 203451 and 209463. By the “the mature Dendriac polypeptide having the amino acid sequence encoded by the cDNA clones contained in ATCC Deposit Nos. 203056 and 209627” is meant the mature form(s) of the Dendriac polypeptides produced by expression in a mammalian cell (e.g., COS cells, as described below) of the complete open reading frames encoded by the human DNA sequence of the deposited clones. Furthermore, by the “the mature Brainiac-3 polypeptide having the amino acid sequence encoded by the cDNA clones contained in ATCC Deposit Nos. 203451 and 209463” is meant the mature form(s) of the Brainiac-3 polypeptides produced by expression in a mammalian cell (e.g., COS cells, as described below) of the complete open reading frames encoded by the human DNA sequence of the deposited clones.

In addition, methods for predicting whether a polypeptide has a secretory leader as well as the cleavage point for that leader sequence are available. For instance, the method of McGeoch (Virus Res. 3:271-286 (1985)) uses the information from a short N-terminal charged region and a subsequent uncharged region of the complete (uncleaved) polypeptide. The method of von Heinje (Nucleic Acids Res. 14:4683-4690 (1986)) uses the information from the residues surrounding the cleavage site, typically residues −13 to +2 where +1 indicates the amino terminus of the mature polypeptide. The accuracy of predicting the cleavage points of known mammalian secretory polypeptides for each of these methods is in the range of 75-80% (von Heinje, supra). However, the two methods do not always produce the same predicted cleavage point(s) for a given polypeptide.

In the present case, the deduced amino acid sequence of the complete Dendriac and Brainiac-3 polypeptides were analyzed by a computer program “SignalP”, (See, Nielsen, H., et al., Prot. Eng. 10:1-6 (1997)), which is an expert system for predicting the cellular location of a polypeptide based on the amino acid sequence. As part of this computational prediction of localization, the methods of von Heinje are incorporated. Thus, the computation analysis above predicted a single signal peptide cleavage site within the complete amino acid sequence shown in SEQ ID NO:2 and SEQ ID NO: 10 and a single signal peptide cleavage site within the complete amino acid sequence shown in SEQ ID NO:4 and SEQ ID NO:12.

The putative signal peptide sequence of the Dendriac polypeptide was predicted by the inventors to be amino acid residues 1-27 of amino acid sequence shown as SEQ ID NO: 10. Subsequently, the signal peptide sequence was predicted by the SignalP computer program to be amino acids 1-25 of the amino acid sequence shown as SEQ ID NO:2 and SEQ ID NO: 10. Accordingly, the putative signal peptide sequence is predicted to be a range between 20 and 30. Thus, preferred polypeptides of the invention comprise any of the following polypeptides: Met-21 to Tyr-319; Trp-22 to Tyr-319; Tyr-23 to Tyr-319; Leu-24 to Tyr-319; Ser-25 to Tyr-319; Leu-26 to Tyr-319; Pro-27 to Tyr-319; His-28 to Tyr-319; Tyr-29 to Tyr-319; and Asn-30 to Tyr-319 of SEQ ID NO:2 and of SEQ ID NO: 10. Polynucleotides encoding these polypeptides are also preferred.

Similarly, the putative signal peptide sequence of the Brainiac-3 polypeptide was predicted by the inventors to be amino acid residues 1-20 of amino acid sequence shown as SEQ ID NO:12. Subsequently, the signal peptide sequence was predicted by the SignalP computer program to be amino acids 1-28 of the amino acid sequence shown as SEQ ID NO:4 and SEQ ID NO:12. Accordingly, the putative signal peptide sequence is predicted to be a range between 15 and 35. Thus, preferred polypeptides of the invention comprise any of the following polypeptides: Val-15 to Arg-352; Leu-16 to Arg-352; Leu-17 to Arg-352; Leu-18 to Arg-352; Gly-19 to Arg-352; Cys-20 to Arg-352; Leu-21 to Arg-352; Leu-22 to Arg-352; Phe-23 to Arg-352; Leu-24 to Arg-352; Arg-25 to Arg-352; Lys-26 to Arg-352; Ala-27 to Arg-352; Ala-28 to Arg-352; Lys-29 to Arg-352; Pro-30 to Arg-352; Ala-31 to Arg-352; Glu-32 to Arg-352; Thr-33 to Arg-352; Pro-34 to Arg-352; and Arg-35 to Arg-352 of SEQ ID NO:4 and of SEQ ID NO:12. Polynucleotides encoding these polypeptides are also preferred.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

In specific embodiments, the polynucleotides of the invention are less than 300 kb, 200 kb, 100 kb, 50 kb, 15 kb, 10 kb or 7.5 kb in length. In a further embodiment, polynucleotides of the invention comprise at least 15 contiguous nucleotides of Dendriac or Brainiac-3 coding sequence, but do not comprise all or a portion of any Dendriac or Brainiac-3 intron. In another embodiment, the nucleic acid comprising Dendriac or Brainiac-3 coding sequence does not contain coding sequences of a genomic flanking gene (i.e., 5′ or 3′ to the Dendriac or Brainiac-3 coding sequences in the genome).

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RINA transcripts of the DNA molecules of the present invention. Isolated nucleic acid molecules according to the present invention further include such molecules produced synthetically. In another embodiment, an “isolated” nucleic acid molecule does not encompass a chromosome isolated or removed from a cell or a cell lysate (e.g., a “chromosome spread”, as in a karyotype).

Isolated nucleic acid molecules of the present invention include DNA molecules comprising an open reading frame (ORF) with an initiation codon at positions 426-428 of the nucleotide sequence shown in FIGS. 1A, 1B, and 1C (SEQ ID NO: 1). In addition, isolated nucleic acid molecules of the present invention include DNA molecules comprising an open reading frame (ORF) with an initiation codon at positions 21-23 of the nucleotide sequence shown in SEQ ID NO:9. Also included are DNA molecules comprising the coding sequence for the predicted mature Dendriac polypeptide shown at positions 1-294 of SEQ ID NO:2 and SEQ ID NO:10.

Isolated nucleic acid molecules of the present invention also include DNA molecules comprising an open reading frame (ORF) with an initiation codon at positions 47-49 of the nucleotide sequence shown in FIGS. 2A and 2B (SEQ ID NO:3) and of SEQ ID NO: 11. Also included are DNA molecules comprising the coding sequence for the predicted mature Brainiac-3 polypeptide shown at positions 1-324 of SEQ ID NOs:2 and 12.

In addition, isolated nucleic acid molecules of the invention include DNA molecules which comprise a sequence substantially different from those described above but which, due to the degeneracy of the genetic code, still encode Dendriac or Brainiac-3 polypeptides of the invention. In specific embodiments, Dendriac or Brainiac-3 variants in which 5-10, 1-5, or 1-2 amino acids are substituted, deleted, or added in any combination are preferred. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another embodiment, the invention provides isolated nucleic acid molecules encoding the Dendriac polypeptide having an amino acid sequence encoded by the cDNA clone contained in the plasmid deposited as ATCC Deposit No.203056 on Jul. 9, 1998. Preferably, this nucleic acid molecule will encode the mature polypeptide encoded by the above-described deposited cDNA clone.

In yet another embodiment, the invention provides isolated nucleic acid molecules encoding the Dendriac polypeptide having an amino acid sequence encoded by the cDNA clone contained in the pooled plasmids deposited as ATCC Deposit No. 209627 on Feb. 12, 1998. Preferably, this nucleic acid molecule will encode the mature polypeptide encoded by the above-described deposited cDNA clone.

The invention further provides an isolated nucleic acid molecule having the nucleotide sequence shown in FIGS. 1A, 1B, and 1C (SEQ ID NO:1) or the nucleotide sequence of the Dendriac cDNA contained in the above-described deposited clone, or a nucleic acid molecule having a sequence complementary to one of the above sequences. The invention still further provides an isolated nucleic acid molecule having the nucleotide sequence shown in SEQ ID NO:9 or the nucleotide sequence of the Dendriac cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the Dendriac gene in human tissue, for instance, by Northern blot analysis.

In another embodiment, the invention provides isolated nucleic acid molecules encoding the Brainiac-3 polypeptide having an amino acid sequence encoded by the cDNA clone contained in the plasmid deposited as ATCC Deposit No. 203451 on Nov. 8, 1998. Preferably, this nucleic acid molecule will encode the mature polypeptide encoded by the above-described deposited cDNA clone.

In still another embodiment, the invention provides isolated nucleic acid molecules encoding the Brainiac-3 polypeptide having an amino acid sequence encoded by the cDNA clone contained in the plasmid deposited as ATCC Deposit No. 209463 on Nov. 14, 1997. Preferably, this nucleic acid molecule will encode the mature polypeptide encoded by the above-described deposited cDNA clone.

The invention further provides an isolated nucleic acid molecule having the nucleotide sequence shown in FIGS. 2A and 2B (SEQ ID NO:3) and in SEQ ID NO: 11 or the nucleotide sequence of the Brainiac-3 cDNA contained in the above-described deposited clones, or a nucleic acid molecule having a sequence complementary to one of the above sequences. Such isolated molecules, particularly DNA molecules, are useful as probes for gene mapping, by in situ hybridization with chromosomes, and for detecting expression of the Brainiac-3 gene in human tissue, for instance, by Northern blot analysis.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO:1 which consists of positions 1-1391 of SEQ ID NO: 1. Also in particular, the invention provides a polynucleotide having a nucleotide sequence representing the portion of SEQ ID NO:9 which consists of positions 1-1773 of SEQ ID NO:9. Further, the invention includes a polynucleotide comprising any portion of at least about 30 nucleotides, preferably at least about 50 nucleotides, of SEQ ID NO: 1 from positions 1 -1391 of SEQ ID NO: 1, excluding the sequences of the following related cDNA clones, and any subfragments therein: HCEPM92RB (SEQ ID NO:6). Also, the invention further includes a polynucleotide comprising any portion of at least about 30 nucleotides, preferably at least about 50 nucleotides, of SEQ ID NO:9 from positions 1-1773 of SEQ ID NO:9, excluding the sequences of the following related cDNA clones, and any subfragments therein: HCEPM92RB (SEQ ID NO:6).

Further, the invention includes a polynucleotide comprising any portion of at least about 25 nucleotides, preferably at least about 30 nucleotides, more preferably at least about 40 nucleotides, and even more preferably at least about 50 nucleotides, of SEQ ID NO:1 from residue 1-2168. More preferably, the invention includes a polynucleotide comprising nucleotides 50-2168, 100-2168, 150-2168, 200-2168, 250-2168, 300-2168, 350-2168, 400-2168, 450-2168, 500-2168, 550-2168, 600-2168, 650-2168, 700-2168, 750-2168, 800-2168, 850-2168, 900-2168, 950-2168, 1000-2168, 1050-2168, 1100-2168, 1150-2168, 1200-2168, 1250-2168, 1300-2168, 1350-2168, 1400-2168, 1450-2168, 1500-2168, 1550-2168, 1600-2168, 1650-2168, 1700-2168, 1750-2168, 1800-2168, 1850-2168, 1900-2168, 1950-2168, 2000-2168, 2050-2168, 2100-2168, 2150-2168, 50-2100, 100-2100, 150-2100, 200-2100, 250-2100, 300-2100, 350-2100, 400-2100, 450-2100,500-2100, 550-2100, 600-2100, 650-2100,700-2100, 750-2100, 800-2100, 850-2100, 900-2100, 950-2100, 1000-2100, 1050-2100, 1100-2100, 1150-2100, 1200-2100, 1250-2100, 1300-2100, 1350-2100, 1400-2100, 1450-2100, 1500-2100, 1550-2100, 1600-2100, 1650-2100, 1700-2100, 1750-2100, 1800-2100, 1850-2100, 1900-2100, 1950-2100, 2000-2100, 2050-2100, 50-2000, 100-2000, 150-2000, 200-2000, 250-2000, 300-2000, 350-2000, 400-2000, 450-2000, 500-2000, 550-2000, 600-2000, 650-2000, 700-2000, 750-2000, 800-2000, 850-2000, 900-2000, 950-2000, 1000-2000, 1050-2000, 1100-2000, 1150-2000, 1200-2000, 1250-2000, 1300-2000, 1350-2000, 1400-2000, 1450-2000, 1500-2000, 1550 -2000, 1600-2000, 1650-2000, 1700-2000, 17 50-2000, 1800-2000, 1850-2000, 1900-2000, 1950-2000, 50-1900, 100-1900, 150-1900, 200-1900, 250-1900, 300-1900, 1350-1900, 400-1900, 450-1900, 500-1900, 550-1900, 600-1900, 650-1900, 700-1900, 750-1900, 800-1900, 850-1900, 900-1900, 950-1900, 1000-1900, 1050-1900, 1100-1900, 1150-1900, 1200-1900, 1250-1900, 1300-1900, 1350-1900, 1400-1900, 1450-1900, 1500-1900, 1550 -1900, 1600-1900, 1650-1900, 1700-1900, 1750-1900, 1800-1900, 1850-1900, 50-1800, 100-1800, 150-1800, 200-1800, 250-1800, 300-1800, 350-1800, 400-1800, 450-1800, 500-1800, 550-1800, 600-1800, 650-1800, 700-1800, 750-1800, 800-1800, 850-1800, 900-1800, 950-1800, 1000-1800, 1050-1800, 1100-1800, 1150-1800, 1200-1800, 1250-1800, 1300-1800, 1350-1800, 1400-1800, 1450-1800, 1500-1800, 1550-1800, 1600-1800, 1650-1800, 1700-1800, 1750-1800, 50-1700, 100-1700, 150-1700, 200-1700, 250-1700, 300-1700, 350-1700, 400-1700, 450-1700, 500-1700, 550-1700, 600-1700, 650-1700, 700-1700, 750-1700, 800-1700, 850-1700, 900-1700, 950-1700, 1000-1700, 1050-1700, 1100-1700, 1150-1700, 1200-1700, 1250-1700, 1300-1700, 1350-1700, 1400-1700, 1450-1700, 1500-1700, 1550-1700, 1600-1700, 1650-1700, 50-1600, 100-1600, 150-1600, 200-1600, 250-1600, 300-1600, 350-1600, 400-1600, 450-1600, 500-1600, 550-1600, 600-1600, 650-1600, 700-1600, 750-1600, 800-1600, 850-1600, 900-1600, 950-1600, 1000-1600, 1050-1600, 1100-1600, 1150-1600, 1200-1600, 1250-1600, 1300-1600, 1350-1600, 1400-1600, 1450-1600, 1500-1600, 1550-1600, 50-1500, 100-1500, 150-1500, 200-1500, 250-1500, 300-1500, 350-1500, 400-1500, 450-1500, 500-1500, 550-1500, 600-1500, 650-1500, 700-1500, 750-1500, 800-1500, 850-1500, 900-1500, 950-1500, 1000-1500, 1050-1500, 1100-1500, 1150-1500, 1200-1500, 1250-1500, 1300-1500, 1350-1500, 1400-1500, 1450-1500, 50-1500, 100-1500, 150-1500, 200-1500, 250-1500, 300-1500, 350-1500, 400-1500, 450-1500, 500-1500, 550-1500, 600-1500, 650-1500, 700-1500, 750-1500, 800-1500, 850-1500, 900-1500, 950-1500, 1000-1500, 1050-1500, 1100-1500, 1150-1500, 1200-1500, 1250-1500, 1300-1500, 1350-1500, 50-1400, 100-1400, 150-1400, 200-1400, 250-1400, 300-1400, 350-1400, 400-1400, 450-1400, 500-1400, 550-1400, 600-1400, 650-1400, 700-1400, 750-1400, 800-1400, 850-1400, 900-1400, 950-1400, 1000-1400, 1050-1400, 1100-1400, 1150-1400, 1200-1400, 1250-1400, 1300-1400, 1350-1400, 50-1300, 100-1300, 150-1300, 200-1300, 250-1300, 300-1300, 350-1300, 400-1300, 450-1300, 500-1300, 550-1300, 600-1300, 650-1300, 700-1300, 750-1300, 800-1300, 850-1300, 900-1300, 950-1300, 1000-1300, 1050-1300, 1100-1300, 1150-1300, 1200-1300, 1250-1300, 50-1200, 100-1200, 150-1200, 200-1200, 250-1200, 300-1200, 350-1200, 400-1200, 450-1200, 500-1200, 550-1200, 600-1200, 650-1200, 700-1200, 750-1200, 800-1200, 850-1200, 900-1200, 950-1200, 1000-1200, 1050-1200, 1100-1200, 1150-1200, 50-1100, 100-1100, 150-1100, 200-1100, 250-1100, 300-1100, 350-1100, 400-1100, 450-1100, 500-1100, 550-1100, 600-1100, 650-1100, 700-1100, 750-1100, 800-1100, 850-1100, 900-1100, 950-1100, 1000-1100, 1050-1100, 50-1000, 100-1000, 150-1000, 200-1000, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 50-900, 100-900, 150-900, 200-900, 250-900, 300-900, 350-900, 400-900, 450-900, 500-900, 550-900, 600-900, 650-900, 700-900, 750-900, 800-900, 850-900, 50-800, 100-800, 150-800, 200-800, 250-800, 300-800, 350-800, 400-800, 450 -800, 500-800, 550-800, 600 -800, 650 -800, 700 -800, 750 -800, 50-700, 100-700, 150-700, 200-700, 250-700, 300-700, 3 50-700, 400-700, 450-700, 500-700, 550-700, 600-700, 650-700, 50-600, 100-600, 150-600, 200-600, 250-600, 300-600, 350-600, 400-600, 450-600, 500-600, 550-600, 50-500, 100-500, 150-500, 200-500, 250-500, 300-500, 350-500, 400-500, 450-500, 50-400, 100-400, 150-400, 200-400, 250-400, 300-400, 350-400, 50-300, 100-300, 150-300, 200-300, 250-300, 50-200, 100-200, 150-200, and 50-100 of SEQ ID NO:1.

Further, the invention includes a polynucleotide comprising any portion of at least about 25 nucleotides, preferably at least about 30 nucleotides, more preferably at least about 40 nucleotides, and even more preferably at least about 50 nucleotides, of SEQ ID NO:9 from residue 1-1773. More preferably, the invention includes a polynucleotide comprising nucleotides 50-1773, 100-1773, 150-1773, 200-1773, 250-1773, 300-1773, 350-1773, 400-1773, 450-1773, 500-1773, 550-1773, 600-1773, 650-1773, 700-1773, 750-1773, 800-1773, 850-1773, 900-1773, 950-1773, 1000-1773, 1050-1773, 1100-1773, 1150-1773, 1200-1773, 1250-1773, 1300-1773, 1350-1773, 1400-1773, 1450-1773, 1500-1773, 1550-1773, 1600-1773, 1650-1773, 1700-1773, 50-1700, 100-1700, 150-1700, 200-1700, 250-1700, 300-1700, 350-1700, 400-1700, 450-1700, 500-1700, 550-1700, 600-1700, 650-1700, 700-1700, 750-1700, 800-1700, 850-1700, 900-1700, 950-1700, 1000-1700, 1050-1700, 1100-1700, 1150-1700, 1200-1700, 1250-1700, 1300-1700, 1350-1700, 1400-1700, 1450-1700, 1500-1700, 1550-1700, 1600-1700, 1650-1700, 50-1600, 100-1600, 150-1600, 200-1600, 250-1600, 300-1600, 350-1600, 400-1600, 450-1600, 500-1600, 550-1600, 600-1600, 650-1600, 700-1600, 750-1600, 800-1600, 850-1600, 900-1600, 950-1600, 1000-1600, 1050-1600, 1100-1600, 1150-1600, 1200-1600, 1250-1600, 1300-1600, 1350-1600, 1400-1600, 1450-1600, 1500-1600, 1550-1600, 50-1500, 100-1500, 150-1500, 200-1500, 250-1500, 300-1500, 350-1500, 400-1500, 450-1500, 500-1500, 550-1500, 600-1500, 650-1500, 700-1500, 750-1500, 800-1500, 850-1500, 900-1500, 950-1500, 1000-1500, 1050-1500, 1100-1500, 1150-1500, 1200-1500, 1250-1500, 1300-1500, 1350-1500, 1400-1500, 1450-1500, 50-1500, 100-1500, 150-1500, 200-1500, 250-1500, 300-1500, 350-1500, 400-1500, 450-1500, 500-1500, 550-1500, 600-1500, 650-1500, 700-1500, 750-1500, 800-1500, 850-1500, 900-1500, 950-1500, 1000-1500, 1050-1500, 1100-1500, 1150-1500, 1200-1500, 1250-1500, 1300-1500, 1350-1500, 50-1400, 100-1400, 150-1400, 200-1400, 250-1400, 300-1400, 350-1400, 400-1400, 450-1400, 500-1400, 550-1400, 600-1400, 650-1400, 700-1400, 750-1400, 800-1400, 850-1400, 900-1400, 950-1400, 1000-1400, 1050-1400, 1100-1400, 1150-1400, 1200-1400, 1250-1400, 1300-1400, 1350-1400, 50-1300, 100-1300, 150-1300, 200-1300, 250-1300, 300-1300, 350-1300, 400-1300, 450-1300, 500-1300, 550-1300, 600-1300, 650-1300, 700-1300, 750-1300, 800-1300, 850-1300, 900-1300, 950-1300, 1000-1300, 1050-1300, 1100-1300, 1150-1300, 1200-1300, 1250-1300, 50-1200, 100-1200, 150-1200, 200-1200, 250-1200, 300-1200, 350-1200, 400-1200, 450-1200, 500-1200, 550-1200, 600-1200, 650-1200, 700-1200, 750-1200, 800-1200, 850-1200, 900-1200, 950-1200, 1000-1200, 1050-1200, 1100-1200, 1150-1200, 50-1100, 100-1100, 150-1100, 200-1100, 250-1100, 300-1100, 350-1100, 400-1100, 450-1100, 500-1100, 550-1100, 600-1100, 650-1100, 700-1100, 750-1100, 800-1100, 850-1100, 900-1100, 950-1100, 1000-1100, 1050-1100, 50-1000, 100-1000, 150-1000, 200-1000, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 50-900, 100-900, 150-900, 200-900, 250-900, 300-900, 350-900, 400-900, 450-900, 500-900, 550-900, 600-900, 650-900, 700-900, 750-900, 800-900, 850-900, 50-800, 100-800, 150-800, 200-800, 250-800, 300-800, 350-800, 400-800, 450 -800, 500-800, 550-800, 600 -800, 650 -800, 700 -800, 750 -800, 50-700, 100-700, 150-700, 200-700, 250-700, 300-700, 350-700, 400-700, 450-700, 500-700, 550-700, 600-700, 650-700, 50-600, 100-600, 150-600, 200-600, 250-600, 300-600, 350-600, 400-600, 450-600, 500-600, 550-600, 50-500, 100-500, 150-500, 200-500, 250-500, 300-500, 350-500, 400-500, 450-500, 50-400, 100-400, 150-400, 200-400, 250-400, 300-400, 350-400, 50-300, 100-300, 150-300, 200-300, 250-300, 50-200, 100-200, 150-200, and 50-100 of SEQ ID NO:9.

Also more preferably, the invention includes a polynucleotide comprising nucleotides 1-50, 51-100, 101-150, 151-200, 201-250, 251-300, 301-350, 351-400, 401-450, 451-500, 501-550, 551-600, 601-650, 651-700, or 701 to the end of SEQ ID NO:9 or the cDNA in the deposited clone. In this context, “about” includes the particularly recited ranges, larger or smaller by several (5, 4, 3, 2, or 1) nucleotides, at either terminus or at both termini. Preferably, these fragments encode a polypeptide which has biological activity.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO:3 which consists of positions 1-1102 of SEQ ID NO:3. Also in particular, the invention provides a polynucleotide having a nucleotide sequence representing the portion of SEQ ID NO: 11 which consists of positions 1 -1271 of SEQ ID NO: 11. Further, the invention includes a polynucleotide comprising any portion of at least about 30 nucleotides, preferably at least about 50 nucleotides, of SEQ ID NO:3 from positions 1-1102 of SEQ ID NO:3, excluding the sequences of the following related cDNA clones, and any subfragments therein: HE2EJ66R (SEQ ID NO:7) and HFCCQ50R (SEQ ID NO:8). Also, the invention includes a polynucleotide comprising any portion of at least about 30 nucleotides, preferably at least about 50 nucleotides, of SEQ ID NO: 11 from positions 1-1271 of SEQ ID NO:11, excluding the sequences of the following related cDNA clones, and any subfragments therein: HE2EJ66R (SEQ ID NO:7) and HFCCQ50R (SEQ ID NO:8).

Moreover, the invention includes a polynucleotide comprising any portion of at least about 25 nucleotides, preferably at least about 30 nucleotides, more preferably at least about 40 nucleotides, and even more preferably at least about 50 nucleotides, of SEQ ID NO:3 from residue 1 to 1253. More preferably, the invention includes a polynucleotide comprising nucleotides 50-1253, 100-1253, 150-1253, 200-1253, 250-1253, 300-1253, 350-1253, 400-1253, 450-1253, 500-1253, 550-1253, 600-1253, 650-1253, 700-1253, 750-1253, 800-1253, 850-1253, 900-1253, 950-1253, 1000-1253, 1050-1253, 1100-1253, 1150-1253, 1200-1253, 50-1200, 100-1200, 150-1200, 200-1200, 250-1200, 300-1200, 350-1200, 400-1200, 450-1200, 500-1200, 550-1200, 600-1200, 650-1200, 700-1200, 750-1200, 800-1200, 850-1200, 900-1200, 950-1200, 1000-1200, 1050-1200, 1100-1200, 1150-1200, 50-1150, 100-1150, 150-1150, 200-1150, 250-1150, 300-1150, 350-1150, 400-1150, 450-1150, 500-1150, 550-1150, 600-1150, 650-1150, 700-1150, 750-1150, 800-1150, 850-1150, 900-1150, 950-1150, 1000-1150, 1050-1150, 1100-1150, 50-1100, 100-1100, 150-1100, 200-1100, 250-1100, 300-1100, 350-1100, 400-1100, 450-1100, 500-1100, 550-1100, 600-1100, 650-1100, 700-1100, 750-1100, 800-1100, 850-1100, 900-1100, 950-1100, 1000-1100, 1050-1100, 50-1050, 100-1050, 150-1050, 200-1050, 250-1050, 300-1050, 350-1050, 400-1050, 450-1050, 500-1050, 550-1050, 600-1050, 650-1050, 700-1050, 750-1050, 800-1050, 850-1050, 900-1050, 950-1050, 1000-1050, 50-1050, 100-1050, 150-1050, 200-1050, 250-1050, 300-1050, 350-1050, 400-1050, 450-1050, 500-1050, 550-1050, 600-1050, 650-1050, 700-1050, 750-1050, 800-1050, 850-1050, 900-1050, 950-1050, 50-1000, 100-1000, 150-1000, 200-1000, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 50-950, 100-950, 150-950, 200-950, 250-950, 300-950, 350-950, 400-950, 450-950, 500-950, 550-950, 600-950, 650-950, 700-950, 750-950, 800-950, 850-950, 900-950, 50-900, 100-900, 150-900, 200-900, 250-900, 300-900, 350-900, 400-900, 450-900, 500-900, 550-900, 600-900, 650-900, 700-900, 750-900, 800-900, 850-900, 50-850, 100-850, 150-850, 200-850, 250-850, 300-850, 350-850, 400-850, 450-850, 500-850, 550-850, 600-850, 650-850, 700-850, 750-850, 800-850, 50-800, 100-800, 150-800, 200-800, 250-800, 300-800, 350-800, 400-800, 450-800, 500-800, 550-800, 600-800, 650-800, 700-800, 750-800, 50-750, 100-750, 150-750, 200-750, 250-750, 300-750, 350-750, 400-750, 450-750, 500-750, 550-750, 600-750, 650-750, 700-750, 50-700, 100-700, 150-700, 200-700, 250-700, 300-700, 350-700, 400-700, 450-700, 500-700, 550-700, 600-700, 650-700, 50-650, 100-650, 150-650, 200-650, 250-650, 300-650, 350-650, 400-650, 450-650, 500-650, 550-650, 600-650, 50-600, 100-600, 150-600, 200-600, 250-600, 300-600, 350-600, 400-600, 450-600, 500-600, 550-600, 50-550, 100-550, 150-550, 200-550, 250-550, 300-550, 350-550, 400-550, 450-550, 500-550, 50-500, 100-500, 150-500, 200-500, 250-500, 300-500, 350-500, 400-500, 450-500, 50-450, 100-450, 150-450, 200-450, 250-450, 300-450, 350-450, 400-450, 50-400, 100-400, 150-400, 200-400, 250-400, 300-400, 350-400, 50-400, 100-400, 150-400, 200-400, 250-400, 300-400, 50-350, 100-350, 150-350, 200-350, 250-350, 300-350, 50-300, 100-300, 150-300, 200-300, 250-300, 50-250, 100-250, 150-250, 200-250, 50-200, 100-200, 150-200, 50-150, 100-150, and 50-100 of SEQ ID NO:3.

Moreover, the invention includes a polynucleotide comprising any portion of at least about 25 nucleotides, preferably at least about 30 nucleotides, more preferably at least about 40 nucleotides, and even more preferably at least about 50 nucleotides, of SEQ ID NO:3 from residue 1 to 1253. More preferably, the invention includes a polynucleotide comprising nucleotides 50-1271, 100-1271, 150-1271, 200-1271, 250-1271, 300-1271, 350-1271, 400-1271, 450-1271, 500-1271, 550-1271, 600-1271, 650-1271, 700-1271, 750-1271, 800-1271, 850-1271, 900-1271, 950-1271, 1000-1271, 1050-1271, 1100-1271, 1150-1271, 1200-1271, 50-1250, 100-1250, 150-1250, 200-1250, 250-1250, 300-1250, 350-1250, 400-1250, 450-1250, 500-1250, 550-1250, 600-1250, 650-1250, 700-1250, 750-1250, 800-1250, 850-1250, 900-1250, 950-1250, 1000-1250, 1050-1250, 1100-1250, 1150-1250, 1200-1250, 50-1200, 100-1200, 150-1200, 200-1200, 250-1200, 300-1200, 350-1200, 400-1200, 450-1200, 500-1200, 550-1200, 600-1200, 650-1200, 700-1200, 750-1200, 800-1200, 850-1200, 900-1200, 950-1200, 1000-1200, 1050-1200, 1100-1200, 1150-1200, 50-1150, 100-1150, 150-1150, 200-1150, 250-1150, 300-1150, 350-1150, 400-1150, 450-1150, 500-1150, 550-1150, 600-1150, 650-1150, 700-1150, 750-1150, 800-1150, 850-1150, 900-1150, 950-1150, 1000-1150, 1050-1150, 1100-1150, 50-1100, 100-1100, 150-1100, 200-1100, 250-1100, 300-1100, 350-1100, 400-1100, 450-1100, 500-1100, 550-1100, 600-1100, 650-1100, 700-1100, 750-1100, 800-1100, 850-1100, 900-1100, 950-1100, 1000-1100, 1050-1100, 50-1050, 100-1050, 150-1050, 200-1050, 250-1050, 300-1050, 350-1050, 400-1050, 450-1050, 500-1050, 550-1050, 600-1050, 650-1050, 700-1050, 750-1050, 800-1050, 850-1050, 900-1050, 950-1050, 1000-1050, 50-1050, 100-1050, 150-1050, 200-1050, 250-1050, 300-1050, 350-1050, 400-1050, 450-1050, 500-1050, 550-1050, 600-1050, 650-1050, 700-1050, 750-1050, 800-1050, 850-1050, 900-1050, 950-1050, 50-1000, 100-1000, 150-1000, 200-1000, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 50-950, 100-950, 150-950, 200-950, 250-950, 300-950, 350-950, 400-950, 450-950, 500-950, 550-950, 600-950, 650-950, 700-950, 750-950, 800-950, 850-950, 900-950, 50-900, 100-900, 150-900, 200-900, 250-900, 300-900, 350-900, 400-900, 450-900, 500-900, 550-900, 600-900, 650-900, 700-900, 750-900, 800-900, 850-900, 50-850, 100-850, 150-850, 200-850, 250-850, 300-850, 350-850, 400-850, 450-850, 500-850, 550-850, 600-850, 650-850, 700-850, 750-850, 800-850, 50-800, 100-800, 150-800, 200-800, 250-800, 300-800, 350-800, 400-800, 450-800, 500-800, 550-800, 600-800, 650-800, 700-800, 750-800, 50-750, 100-750, 150-750, 200-750, 250-750, 300-750, 350-750, 400-750, 450-750, 500-750, 550-750, 600-750, 650-750, 700-750, 50-700, 100-700, 150-700, 200-700, 250-700, 300-700, 350-700, 400-700, 450-700, 500-700, 550-700, 600-700, 650-700, 50-650, 100-650, 150-650, 200-650, 250-650, 300-650, 350-650, 400-650, 450-650, 500-650, 550-650, 600-650, 50-600, 100-600, 150-600, 200-600, 250-600, 300-600, 350-600, 400-600, 450-600, 500-600, 550-600, 50-550, 100-550, 150-550, 200-550, 250-550, 300-550, 350-550, 400-550, 450-550, 500-550, 50-500, 100-500, 150-500, 200-500, 250-500, 300-500, 350-500, 400-500, 450-500, 50-450, 100-450, 150-450, 200-450, 250-450, 300-450, 350-450, 400-450, 50-400, 100-400, 150-400, 200-400, 250-400, 300-400, 350-400, 50-400, 100-400, 150-400, 200-400, 250-400, 300-400, 50-350, 100-350, 150-350, 200-350, 250-350, 300-350, 50-300, 100-300, 150-300, 200-300, 250-300, 50-250, 100-250, 150-250, 200-250, 50-200, 100-200, 150-200, 50-150, 100-150, and 50-100 of SEQ ID NO:11.

More generally, by a fragment of an isolated nucleic acid molecule having the nucleotide sequence of the deposited cDNAs or the nucleotide sequences shown in FIGS. 1A, 1B, and 1C (SEQ ID NO:1) and SEQ ID NO:9, and as shown in FIGS. 2A and 2B (SEQ ID NO:3) and SEQ ID NO: 11, is intended fragments at least about 15 nt, and more preferably at least about 20 nt, still more preferably at least about 30 nt, and even more preferably, at least about 40, 50, 60, 70, 80, 90, 100, 150, 200, 250, 300, 400, or 500 nt in length which are useful as diagnostic probes and primers as discussed herein. Of course, larger fragments 50-300 nt in length are also useful according to the present invention as are fragments corresponding to most, if not all, of the nucleotide sequence of the deposited cDNAs or as shown in FIGS. 1A, 1B, and 1C (SEQ ID NO:1) and SEQ ID NO:9, and as shown in FIGS. 2A and 2B (SEQ ID NO:3) and SEQ ID NO:11. By a fragment at least 20 nt in length, for example, is intended fragments which include 20 or more contiguous bases from the nucleotide sequence of the deposited cDNAs or the nucleotide sequence as shown in FIGS. 1A, 1B, and 1C (SEQ ID NO:1) and SEQ ID NO:9, and as shown in FIGS. 2A and 2B (SEQ ID NO:3) and SEQ ID NO:11. By about in the phrase at least about is meant several, a few, a small number, 5, 4, 3, 2 or 1. Preferred nucleic acid fragments of the present invention include nucleic acid molecules encoding epitope-bearing portions of the Dendriac or Brainiac-3 polypeptides as identified in FIGS. 4 and 5, respectively, and described in more detail below.

In additional embodiments, the polynucleotides of the invention encode functional attributes of Dendriac or Brainiac-3. Preferred embodiments of the invention in this regard include fragments that comprise alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of Dendriac or Brainiac-3 .

The data representing the structural or functional attributes of Dendriac or Brainiac-3 set forth in FIGS. 4 and 5 and/or Tables I and II, as described above, was generated using the various modules and algorithms of the DNA*STAR set on default parameters. In a preferred embodiment, the data presented in columns VIII, IX, XIII, and XIV of Tables I and II can be used to determine regions of Dendriac or Brainiac-3 which exhibit a high degree of potential for antigenicity. Regions of high antigenicity are determined from the data presented in columns VIII, IX, XIII, and/or IV by choosing values which represent regions of the polypeptide which are likely to be exposed on the surface of the polypeptide in an environment in which antigen recognition may occur in the process of initiation of an immune response.

Certain preferred regions in these regards are set out in FIGS. 4 and 5, but may, as shown in Tables I and II, respectively, be represented or identified by using tabular representations of the data presented in FIGS. 4 and 5. The DNA*STAR computer algorithm used to generate FIGS. 4 and 5 (set on the original default parameters) was used to present the data in FIGS. 4 and 5 in a tabular format (See Tables I and II, respectively). The tabular format of the data in FIG. 4 or in FIG. 5 may be used to easily determine specific boundaries of a preferred region.

The above-mentioned preferred regions set out in FIGS. 4 and 5 and in Tables I and II include, but are not limited to, regions of the aforementioned types identified by analysis of the amino acid sequence set out in FIGS. 1A, 1B, and 1C, and in FIGS. 2A and 2B. As set out in FIGS. 4 and 5 and in Tables I and II, such preferred regions include Gamier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index.

TABLE I

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | A | . | . | . | . | . | . | 0.04 | −0.03 | * | * | . | 0.50 | 0.80 |
| Ser | 2 | A | . | . | . | . | . | . | −0.38 | −0.07 | * | . | . | 0.50 | 0.84 |
| Leu | 3 | A | . | . | . | . | . | . | 0.06 | 0.19 | * | . | . | −0.10 | 0.54 |
| Arg | 4 | A | . | . | . | . | . | . | 0.16 | −0.24 | * | . | . | 0.65 | 1.10 |
| Ser | 5 | A | . | . | B | . | . | . | 0.24 | 0.06 | * | * | F | −0.15 | 0.86 |
| Leu | 6 | A | . | . | B | . | . | . | 0.03 | 0.06 | . | . | . | −0.15 | 1.40 |
| Lys | 7 | A | . | . | B | . | . | . | −0.48 | 0.06 | . | * | . | −0.30 | 0.59 |
| Trp | 8 | A | . | . | B | . | . | . | −0.48 | 0.74 | * | . | . | −0.60 | 0.36 |
| Ser | 9 | A | . | . | B | . | . | . | −1.40 | 1.04 | * | . | . | −0.60 | 0.36 |
| Leu | 10 | . | . | B | B | . | . | . | −1.40 | 1.04 | . | * | . | −0.60 | 0.15 |
| Leu | 11 | . | . | B | B | . | . | . | −1.40 | 1.43 | . | * | . | −0.60 | 0.19 |
| Leu | 12 | . | . | B | B | . | . | . | −2.26 | 1.20 | * | . | . | −0.60 | 0.12 |
| Leu | 13 | A | . | . | B | . | . | . | −2.27 | 1.50 | . | . | . | −0.60 | 0.12 |
| Ser | 14 | A | . | . | B | . | . | . | −2.67 | 1.20 | . | . | . | −0.60 | 0.19 |
| Leu | 15 | A | . | . | B | . | . | . | −2.56 | 1.30 | . | . | . | −0.60 | 0.20 |
| Leu | 16 | A | . | . | B | . | . | . | −2.60 | 1.40 | . | . | . | −0.60 | 0.21 |
| Ser | 17 | A | . | . | B | . | . | . | −2.39 | 1.36 | . | . | . | −0.60 | 0.12 |
| Phe | 18 | . | . | B | B | . | . | . | −1.87 | 1.59 | . | . | . | −0.60 | 0.14 |
| Phe | 19 | . | . | B | B | . | . | . | −1.81 | 1.81 | . | . | . | −0.60 | 0.18 |
| Val | 20 | . | . | B | B | . | . | . | −1.81 | 1.89 | . | . | . | −0.60 | 0.21 |
| Met | 21 | . | . | B | B | . | . | . | −1.30 | 2.19 | . | * | . | −0.60 | 0.20 |
| Trp | 22 | . | . | B | B | . | . | . | −1.81 | 1.79 | . | . | . | −0.60 | 0.31 |
| Tyr | 23 | . | . | B | B | . | . | . | −1.32 | 1.69 | . | . | . | −0.60 | 0.34 |
| Leu | 24 | . | . | . | B | T | . | . | −0.66 | 1.47 | . | . | . | −0.20 | 0.53 |
| Ser | 25 | . | . | . | B | . | . | C | −0.04 | 1.36 | . | . | . | −0.40 | 0.69 |
| Leu | 26 | . | . | . | . | . | T | C | 0.56 | 1.20 | . | . | . | 0.00 | 0.69 |
| Pro | 27 | . | . | . | . | . | T | C | −0.01 | 0.84 | . | . | . | 0.15 | 1.34 |
| His | 28 | . | . | . | . | T | T | . | −0.66 | 0.80 | . | . | . | 0.20 | 0.74 |
| Tyr | 29 | . | . | . | . | . | T | C | 0.16 | 1.10 | * | . | . | 0.00 | 0.63 |
| Asn | 30 | . | . | B | B | . | . | . | 0.57 | 0.41 | * | * | . | −0.60 | 0.71 |
| Val | 31 | . | . | B | B | . | . | . | 0.52 | −0.01 | * | * | . | 0.45 | 1.02 |
| Ile | 32 | . | . | B | B | . | . | . | 0.73 | 0.13 | * | * | . | −0.30 | 0.48 |
| Glu | 33 | . | . | B | B | . | . | . | 0.48 | −0.23 | * | * | . | 0.30 | 0.48 |
| Arg | 34 | . | . | B | B | . | . | . | 0.12 | 0.29 | * | * | . | −0.30 | 0.68 |
| Val | 35 | . | . | B | B | . | . | . | −0.12 | 0.26 | * | * | . | −0.30 | 0.97 |
| Asn | 36 | . | . | . | B | T | . | . | 0.03 | 0.33 | * | * | . | 0.10 | 0.87 |
| Trp | 37 | . | . | B | B | . | . | . | 0.68 | 1.11 | . | . | . | −0.60 | 0.39 |
| Met | 38 | . | . | B | B | . | . | . | 0.68 | 1.87 | . | * | . | −0.60 | 0.82 |
| Tyr | 39 | . | . | B | B | . | . | . | 0.32 | 1.23 | . | * | . | −0.60 | 0.88 |
| Phe | 40 | . | . | B | B | . | . | . | 1.18 | 1.59 | . | . | . | −0.45 | 1.31 |
| Tyr | 41 | . | . | B | . | . | . | . | 0.97 | 0.67 | . | . | . | −0.25 | 2.29 |
| Glu | 42 | . | . | . | . | T | . | . | 0.37 | 0.49 | . | . | . | 0.15 | 2.26 |
| Tyr | 43 | . | . | . | . | T | . | . | 0.72 | 0.41 | * | . | . | 0.15 | 1.83 |
| Glu | 44 | . | . | . | . | . | . | C | 1.08 | 0.39 | * | . | . | 0.25 | 1.83 |
| Pro | 45 | . | . | . | . | T | . | . | 1.78 | −0.37 | * | . | . | 1.05 | 2.07 |
| Ile | 46 | . | . | . | . | T | . | . | 2.02 | 0.03 | . | * | . | 0.45 | 2.29 |
| Tyr | 47 | A | . | . | . | . | . | . | 1.32 | −0.73 | * | * | F | 1.10 | 2.21 |
| Arg | 48 | A | . | . | . | . | . | . | 1.53 | 0.06 | * | * | F | 0.20 | 1.24 |
| Gln | 49 | A | . | . | . | . | . | . | 0.83 | 0.13 | * | * | F | 0.20 | 2.40 |
| Asp | 50 | A | . | . | . | . | . | . | 0.73 | 0.23 | . | * | F | 0.20 | 1.32 |
| Phe | 51 | A | . | . | B | . | . | . | 0.81 | −0.04 | * | * | . | 0.30 | 0.98 |
| His | 52 | . | . | B | B | . | . | . | 1.17 | 0.64 | * | * | . | −0.60 | 0.46 |
| Phe | 53 | . | . | B | B | . | . | . | 1.06 | 0.24 | * | * | . | −0.30 | 0.55 |
| Thr | 54 | . | . | B | B | . | . | . | 1.02 | 0.24 | * | * | . | −0.15 | 1.09 |
| Leu | 55 | A | . | . | B | . | . | . | 0.72 | −0.04 | * | * | . | 0.76 | 1.09 |
| Arg | 56 | A | . | . | B | . | . | . | 1.42 | −0.16 | * | * | . | 1.07 | 1.69 |
| Glu | 57 | . | . | . | . | T | . | . | 0.79 | −0.54 | . | * | F | 2.43 | 1.88 |
| His | 58 | . | . | . | . | T | T | . | 1.19 | −0.46 | . | * | F | 2.64 | 1.22 |
| Ser | 59 | . | . | . | . | T | T | . | 1.47 | −0.76 | * | * | F | 3.10 | 0.84 |
| Asn | 60 | . | . | . | . | T | T | . | 2.28 | −0.26 | * | * | . | 2.34 | 0.66 |
| Cys | 61 | . | . | . | . | T | T | . | 2.17 | 0.14 | . | . | . | 1.43 | 0.84 |
| Ser | 62 | . | . | . | . | T | . | . | 1.96 | 0.04 | . | . | . | 1.07 | 1.00 |
| His | 63 | . | . | . | . | T | . | . | 1.29 | 0.09 | . | . | F | 0.76 | 0.96 |
| Gln | 64 | . | . | . | . | . | . | C | 0.78 | 0.47 | . | . | F | 0.10 | 1.56 |
| Asn | 65 | . | . | . | . | . | T | C | −0.08 | 0.59 | . | . | F | 0.15 | 0.96 |
| Pro | 66 | . | . | B | . | . | T | . | −0.30 | 0.84 | . | . | F | −0.05 | 0.52 |
| Phe | 67 | . | . | B | . | . | T | . | −0.81 | 1.03 | . | . | . | −0.20 | 0.21 |
| Leu | 68 | . | . | B | . | . | T | . | −1.63 | 1.31 | . | . | . | −0.20 | 0.11 |
| Val | 69 | . | . | B | B | . | . | . | −1.94 | 1.56 | . | . | . | −0.60 | 0.05 |
| Ile | 70 | . | . | B | B | . | . | . | −2.24 | 1.61 | . | . | . | −0.60 | 0.09 |
| Leu | 71 | . | . | B | B | . | . | . | −2.07 | 1.21 | . | . | . | −0.60 | 0.14 |
| Val | 72 | . | . | B | B | . | . | . | −1.58 | 1.03 | . | . | . | −0.60 | 0.26 |
| Thr | 73 | . | . | B | B | . | . | . | −1.07 | 0.81 | . | . | . | −0.26 | 0.57 |
| Ser | 74 | . | . | . | B | . | . | C | −0.21 | 0.51 | . | . | F | 0.43 | 0.93 |
| His | 75 | . | . | . | . | . | T | C | −0.18 | −0.17 | . | * | F | 2.22 | 2.09 |
| Pro | 76 | . | . | . | . | . | T | C | 0.68 | −0.17 | . | * | F | 2.56 | 1.07 |
| Ser | 77 | . | . | . | . | T | T | . | 0.94 | −0.66 | . | * | F | 3.40 | 1.60 |
| Asp | 78 | A | . | . | . | . | T | . | 1.27 | −0.54 | . | * | F | 2.66 | −1.19 |
| Val | 79 | A | . | . | . | . | . | . | 1.67 | −1.04 | . | * | F | 2.12 | 1.51 |
| Lys | 80 | A | . | . | . | . | . | . | 1.11 | −1.07 | . | * | F | 1.78 | 1.95 |
| Ala | 81 | A | . | . | . | . | . | . | 0.43 | −0.96 | * | * | F | 1.44 | 1.18 |
| Arg | 82 | A | . | . | B | . | . | . | 0.84 | −0.27 | * | * | F | 0.60 | 1.11 |
| Gln | 83 | . | . | B | B | . | . | . | −0.01 | −0.91 | * | * | . | 0.75 | 1.09 |
| Ala | 84 | . | . | B | B | . | . | . | 0.53 | −0.27 | * | * | . | 0.30 | 0.80 |
| Ile | 85 | . | . | B | B | . | . | . | 0.20 | −0.29 | * | * | . | 0.30 | 0.59 |
| Arg | 86 | . | . | B | B | . | . | . | 0.44 | 0.63 | * | * | . | −0.60 | 0.36 |
| Val | 87 | . | . | B | B | . | . | . | 0.33 | 0.66 | * | * | . | −0.30 | 0.35 |
| Thr | 88 | . | . | B | B | . | . | . | 0.38 | 0.16 | * | * | . | 0.30 | 0.87 |
| Trp | 89 | . | . | . | B | . | . | C | 1.01 | −0.53 | . | * | . | 1.70 | 0.88 |
| Gly | 90 | . | . | . | B | . | . | C | 1.60 | −0.53 | * | * | F | 2.30 | 2.38 |
| Glu | 91 | . | . | . | . | T | . | . | 1.20 | −0.79 | . | * | F | 3.00 | 2.21 |
| Lys | 92 | . | . | . | . | T | T | . | 1.77 | −0.36 | . | . | F | 2.60 | 2.21 |
| Lys | 93 | . | . | . | . | T | T | . | 1.73 | −0.36 | . | . | F | 2.30 | 2.35 |
| Ser | 94 | . | . | . | . | T | T | . | 1.78 | −0.36 | . | . | F | 2.00 | 1.34 |
| Trp | 95 | . | . | . | . | T | T | . | 2.12 | 0.40 | . | . | . | 0.65 | 1.05 |
| Trp | 96 | . | . | . | B | . | . | C | 1.27 | 0.40 | . | . | . | −0.40 | 0.91 |
| Gly | 97 | . | . | . | B | . | . | C | 0.41 | 1.04 | . | . | . | −0.40 | 0.51 |
| Tyr | 98 | . | A | B | B | . | . | . | 0.06 | 1.34 | . | . | . | −0.60 | 0.40 |
| Glu | 99 | . | A | B | B | . | . | . | −0.34 | 0.91 | . | . | . | −0.60 | 0.54 |
| Val | 100 | . | A | B | B | . | . | . | −0.76 | 0.79 | . | . | . | −0.60 | 0.48 |
| Leu | 101 | . | A | B | B | . | . | . | −1.28 | 1.14 | . | . | . | −0.60 | 0.26 |
| Thr | 102 | . | A | B | B | . | . | . | −1.74 | 1.07 | . | . | . | −0.60 | 0.13 |
| Phe | 103 | . | A | B | B | . | . | . | −1.84 | 1.76 | . | . | . | −0.60 | 0.14 |
| Phe | 104 | A | A | . | B | . | . | . | −1.84 | 1.54 | . | . | . | −0.60 | 0.17 |
| Leu | 105 | A | A | . | B | . | . | . | −0.99 | 1.26 | . | . | . | −0.60 | 0.20 |
| Leu | 106 | A | A | . | B | . | . | . | −0.77 | 0.77 | . | . | . | −0.60 | 0.40 |
| Gly | 107 | A | A | . | B | . | . | . | −0.46 | 0.49 | * | . | F | −0.45 | 0.47 |
| Gin | 108 | A | A | . | . | . | . | . | 0.29 | −0.30 | * | . | F | 0.45 | 0.98 |
| Glu | 109 | A | A | . | . | . | . | . | 0.99 | −0.99 | . | . | F | 0.90 | 2.38 |
| Ala | 110 | A | A | . | . | . | . | . | 1.80 | −1.67 | * | . | F | 0.90 | 4.17 |
| Glu | 111 | A | A | . | . | . | . | . | 2.66 | −2.10 | * | . | F | 0.90 | 4.02 |
| Lys | 112 | A | A | . | . | . | . | . | 2.40 | −2.50 | * | . | F | 0.90 | 4.64 |
| Glu | 113 | A | A | . | . | . | . | . | 1.59 | −1.89 | * | . | F | 0.90 | 4.54 |
| Asp | 114 | A | A | . | . | . | . | . | 1.00 | −1.70 | . | . | F | 0.90 | 2.16 |
| Lys | 115 | A | A | . | . | . | . | . | 0.78 | −1.20 | . | . | F | 0.90 | 1.09 |
| Met | 116 | A | A | . | . | . | . | . | 0.48 | −0.51 | . | . | . | 0.60 | 0.52 |
| Leu | 117 | A | A | . | . | . | . | . | −0.38 | −0.13 | . | . | . | 0.30 | 0.42 |
| Ala | 118 | A | A | . | . | . | . | . | −0.38 | 0.56 | . | . | . | −0.60 | 0.17 |
| Leu | 119 | A | A | . | . | . | . | . | −0.38 | 0.56 | . | . | . | −0.60 | 0.30 |
| Ser | 120 | A | A | . | . | . | . | . | −0.42 | −0.06 | . | . | . | 0.30 | 0.61 |
| Leu | 121 | A | A | . | . | . | . | . | 0.14 | −0.74 | . | . | . | 0.75 | 1.05 |
| Glu | 122 | A | A | . | . | . | . | . | 0.14 | −0.74 | . | . | . | 0.75 | 1.73 |
| Asp | 123 | A | A | . | . | . | . | . | −0.08 | −0.74 | . | . | F | 0.90 | 1.06 |
| Glu | 124 | A | A | . | . | . | . | . | 0.49 | −0.44 | . | . | . | 0.45 | 1.06 |
| Bis | 125 | A | A | . | B | . | . | . | 0.44 | −0.37 | . | . | . | 0.30 | 0.96 |
| Leu | 126 | A | A | . | B | . | . | . | 1.26 | 0.06 | . | . | . | −0.30 | 0.57 |
| Leu | 127 | A | A | . | B | . | . | . | 0.37 | 0.06 | . | . | . | −0.30 | 0.55 |
| Tyr | 128 | A | A | . | B | . | . | . | −0.52 | 0.74 | * | . | . | −0.60 | 0.28 |
| Gly | 129 | A | . | . | B | . | . | . | −0.41 | 0.93 | * | . | . | −0.60 | 0.24 |
| Asp | 130 | A | . | . | B | . | . | . | −0.38 | 0.24 | * | . | . | −0.30 | 0.57 |
| Ile | 131 | . | . | B | B | . | . | . | 0.43 | −0.04 | * | . | . | 0.30 | 0.63 |
| Ile | 132 | . | . | B | B | . | . | . | 0.54 | −0.80 | * | . | . | 0.75 | 1.07 |
| Arg | 133 | . | . | B | B | . | . | . | −0.02 | −0.44 | * | . | F | 0.45 | −0.55 |
| Gln | 134 | . | . | B | B | . | . | . | 0.32 | 0.24 | * | . | F | −0.15 | 0.65 |
| Asp | 135 | . | . | B | B | . | . | . | 0.01 | −0.44 | * | . | F | 0.60 | 1.55 |
| Phe | 136 | . | . | B | B | . | . | . | 0.66 | −0.64 | * | . | F | 0.90 | 1.14 |
| Leu | 137 | . | . | B | B | . | . | . | 1.54 | 0.11 | * | * | F | 0.00 | 1.03 |
| Asp | 138 | . | . | . | . | T | T | . | 1.43 | 0.11 | * | . | F | 0.65 | 0.99 |
| Thr | 139 | . | . | . | . | T | T | . | 0.62 | 0.51 | * | . | F | 0.50 | 1.85 |
| Tyr | 140 | . | . | . | . | T | T | . | 0.31 | 0.41 | * | . | F | 0.50 | 1.85 |
| Asn | 141 | . | . | . | . | T | T | . | 0.20 | 0.21 | * | * | . | 0.65 | 1.60 |
| Asn | 142 | A | . | . | B | . | . | . | 1.06 | 0.90 | * | * | . | −0.60 | 0.91 |
| Leu | 143 | A | . | . | B | . | . | . | 0.74 | 0.41 | * | . | . | −0.45 | 1.16 |
| Thr | 144 | A | . | . | B | . | . | . | 0.17 | 0.14 | . | . | . | −0.15 | 1.04 |
| Leu | 145 | A | . | . | B | . | . | . | −0.19 | 0.43 | . | . | . | −0.60 | 0.46 |
| Lys | 146 | . | . | B | B | . | . | . | −0.78 | 0.64 | . | * | . | −0.60 | 0.55 |
| Thr | 147 | . | . | B | B | . | . | . | −1.48 | 0.46 | * | * | . | −0.60 | 0.38 |
| Ile | 148 | . | . | B | B | . | . | . | −0.56 | 0.76 | . | * | . | −0.60 | 0.40 |
| Met | 149 | . | . | B | B | . | . | . | −0.53 | 0.07 | . | * | . | −0.30 | 0.39 |
| Ala | 150 | A | . | . | B | . | . | . | −0.58 | 0.99 | . | * | . | −0.60 | 0.29 |
| Phe | 151 | A | . | . | B | . | . | . | −0.93 | 1.14 | * | * | . | −0.60 | 0.30 |
| Arg | 152 | A | . | . | B | . | . | . | −0.62 | 0.94 | * | * | . | −0.60 | 0.44 |
| Trp | 153 | . | . | B | B | . | . | . | −0.43 | 0.33 | * | * | . | −0.30 | 0.76 |
| Val | 154 | A | . | . | B | . | . | . | −0.50 | 0.61 | * | * | . | −0.60 | 0.76 |
| Thr | 155 | A | . | . | B | . | . | . | −0.12 | 0.40 | * | * | . | −0.30 | 0.21 |
| Glu | 156 | . | . | . | B | T | . | . | 0.58 | 0.83 | * | * | . | −0.20 | 0.31 |
| Phe | 157 | . | . | . | . | T | . | . | −0.12 | 0.31 | * | * | . | 0.30 | 0.66 |
| Cys | 158 | . | . | . | . | . | T | C | 0.21 | 0.17 | * | * | . | 0.30 | 0.46 |
| Pro | 159 | . | . | . | . | T | T | . | 0.82 | −0.31 | * | * | . | 1.10 | 0.53 |
| Asn | 160 | A | . | . | . | . | T | . | 0.28 | 0.44 | * | * | F | −0.05 | 0.97 |
| Ala | 161 | A | . | . | . | . | T | . | −0.32 | 0.30 | * | * | F | 0.40 | 1.34 |
| Lys | 162 | A | . | . | B | . | . | . | 0.42 | 0.34 | * | * | . | −0.30 | 0.86 |
| Tyr | 163 | A | . | . | B | . | . | . | 0.78 | −0.09 | . | * | . | 0.63 | 1.07 |
| Val | 164 | . | . | B | B | . | . | . | 0.99 | −0.00 | . | * | . | 0.81 | 1.52 |
| Met | 165 | . | . | B | B | . | . | . | 0.68 | −0.50 | * | * | . | 1.29 | 1.27 |
| Lys | 166 | . | . | B | B | . | . | . | 1.27 | −0.01 | * | * | F | 1.32 | 1.17 |
| Thr | 167 | . | . | B | B | . | . | . | 0.37 | −0.77 | * | * | F | 1.80 | 2.64 |
| Asp | 168 | . | . | B | . | . | T | . | −0.09 | −0.77 | . | * | F | 2.02 | 1.98 |
| Thr | 169 | . | . | B | . | . | T | . | −0.12 | −0.60 | * | * | F | 1.69 | 0.86 |
| Asp | 170 | . | . | B | . | . | T | . | 0.48 | 0.09 | * | * | F | 0.61 | 0.42 |
| Val | 171 | . | . | B | . | . | T | . | 0.12 | −0.00 | . | * | . | 0.88 | 0.40 |
| Phe | 172 | . | . | B | . | . | . | . | 0.09 | 0.49 | . | . | . | −0.40 | 0.40 |
| Ile | 173 | . | . | B | . | . | . | . | 0.09 | 0.43 | . | * | . | −0.40 | 0.24 |
| Asn | 174 | . | . | B | . | . | T | . | −0.41 | 0.83 | * | . | . | −0.20 | 0.51 |
| Thr | 175 | . | . | . | . | . | T | C | −1.27 | 0.87 | * | . | F | 0.15 | 0.49 |
| Gly | 176 | . | . | . | . | T | T | . | −0.37 | 0.73 | * | . | F | 0.35 | 0.52 |
| Asn | 177 | . | . | . | . | . | T | C | 0.09 | 0.04 | * | . | F | 0.45 | 0.64 |
| Leu | 178 | . | . | B | B | . | . | . | 0.17 | 0.40 | * | . | F | −0.15 | 0.70 |
| Val | 179 | . | . | B | B | . | . | . | −0.64 | 0.60 | * | . | . | −0.60 | 0.58 |
| Lys | 180 | . | . | B | B | . | . | . | −0.33 | 0.86 | * | * | . | −0.60 | 0.30 |
| Tyr | 181 | . | . | B | B | . | . | . | −0.80 | 0.86 | * | * | . | −0.60 | 0.58 |
| Leu | 182 | . | . | B | B | . | . | . | −0.80 | 0.86 | * | * | . | −0.60 | 0.65 |
| Leu | 183 | . | . | B | B | . | . | . | −0.02 | 0.61 | * | * | . | −0.60 | 0.52 |
| Asn | 184 | A | . | . | B | . | . | . | 0.53 | 1.11 | * | * | . | −0.60 | 0.45 |
| Leu | 185 | A | . | . | B | . | . | . | 0.49 | 0.74 | * | * | . | −0.60 | 0.74 |
| Asn | 186 | A | . | . | B | . | . | . | 0.78 | 0.06 | . | * | . | −0.15 | 1.54 |
| His | 187 | A | . | . | . | . | . | . | 0.89 | −0.63 | . | * | . | 0.95 | 1.92 |
| Ser | 188 | . | . | . | . | . | . | C | 1.00 | −0.24 | . | * | F | 1.00 | 2.02 |
| Glu | 189 | A | . | . | . | . | . | . | 0.69 | −0.14 | * | . | F | 0.80 | 1.09 |
| Lys | 190 | . | . | B | B | . | . | . | 1.16 | −0.06 | * | . | F | 0.60 | 1.15 |
| Phe | 191 | . | . | B | B | . | . | . | 0.91 | −0.13 | * | . | F | 0.45 | 0.85 |
| Phe | 192 | . | . | B | B | . | . | . | 0.73 | 0.24 | . | . | . | −0.30 | 0.77 |
| Thr | 193 | . | . | B | B | . | . | . | 0.22 | 0.67 | * | . | . | −0.60 | 0.59 |
| Gly | 194 | . | . | B | B | . | . | . | −0.67 | 1.36 | . | . | . | −0.60 | 0.57 |
| Tyr | 195 | . | . | B | B | . | . | . | −0.71 | 1.26 | * | . | . | −0.60 | 0.46 |
| Pro | 196 | . | . | . | B | . | . | C | −0.01 | 0.47 | * | . | . | −0.40 | 0.53 |
| Leu | 197 | . | . | . | B | T | . | . | 0.44 | 0.39 | . | . | . | 0.10 | 0.86 |
| Ile | 198 | . | . | B | B | . | . | . | 0.46 | 0.71 | . | . | . | −0.60 | 0.86 |
| Asp | 199 | . | . | B | B | . | . | . | 0.56 | 0.34 | * | . | . | −0.30 | 0.75 |
| Asn | 200 | . | . | B | . | . | T | . | 0.91 | 0.67 | * | * | . | −0.05 | 1.42 |
| Tyr | 201 | . | . | B | . | . | T | . | 0.78 | −0.01 | * | . | . | 0.85 | 3.97 |
| Ser | 202 | . | . | B | . | . | T | . | 0.89 | −0.27 | * | . | . | 0.85 | 2.35 |
| Tyr | 203 | . | . | . | . | T | T | . | 1.53 | 0.51 | * | * | . | 0.35 | 1.27 |
| Arg | 204 | . | . | . | B | T | . | . | 1.53 | 0.87 | . | . | . | −0.05 | 1.27 |
| Gly | 205 | . | . | . | B | T | . | . | 1.58 | 0.51 | * | * | . | −0.05 | 1.64 |
| Phe | 206 | . | . | B | B | . | . | . | 1.51 | 0.13 | * | . | . | −0.15 | 2.09 |
| Tyr | 207 | . | . | B | B | . | . | . | 1.78 | −0.14 | * | * | . | 0.45 | 1.54 |
| Gln | 208 | . | . | B | B | . | . | . | 1.13 | 0.36 | * | * | F | 0.00 | 2.12 |
| Lys | 209 | . | . | B | B | . | . | . | 0.72 | 0.61 | . | * | F | −0.30 | 1.71 |
| Thr | 210 | . | . | B | B | . | . | . | 0.82 | 0.21 | . | * | F | 0.00 | 1.46 |
| His | 211 | . | . | B | B | . | . | . | 1.52 | 0.21 | . | * | . | −0.15 | 1.33 |
| Ile | 212 | . | . | B | B | . | . | . | 1.77 | 0.21 | . | . | . | −0.15 | 1.15 |
| Ser | 213 | . | . | B | B | . | . | . | 1.52 | 0.21 | . | . | . | 0.15 | 1.38 |
| Tyr | 214 | . | . | B | B | . | . | . | 1.27 | 0.49 | . | * | . | −0.45 | 1.59 |
| Gln | 215 | . | . | B | B | . | . | . | 0.88 | 0.41 | . | * | . | −0.45 | 3.50 |
| Gln | 216 | . | . | B | B | . | . | . | 0.96 | 0.51 | . | * | . | −0.45 | 2.26 |
| Tyr | 217 | . | . | B | . | . | T | . | 0.99 | 0.13 | * | . | . | 0.25 | 2.88 |
| Pro | 218 | . | . | . | . | T | T | . | 0.59 | 0.01 | * | . | . | 0.65 | 1.24 |
| Phe | 219 | . | . | . | . | T | T | . | 0.62 | 0.40 | * | . | . | 0.50 | 0.62 |
| Lys | 220 | . | . | B | . | . | T | . | 0.41 | 0.83 | * | . | . | −0.20 | 0.61 |
| Val | 221 | . | . | B | . | . | . | . | 0.17 | 0.50 | * | * | . | −0.40 | 0.61 |
| Phe | 222 | . | . | B | . | . | . | . | −0.26 | 0.83 | * | . | . | −0.25 | 1.10 |
| Pro | 223 | . | . | B | . | . | T | . | −0.34 | 0.61 | * | . | . | −0.20 | 0.30 |
| Pro | 224 | . | . | . | . | T | T | . | 0.01 | 1.00 | * | . | . | 0.20 | 0.53 |
| Tyr | 225 | . | . | . | . | T | T | . | −0.84 | 0.79 | * | . | . | 0.20 | 0.61 |
| Cys | 226 | . | . | . | . | T | T | . | −0.33 | 0.69 | . | . | . | 0.20 | 0.33 |
| Ser | 227 | . | . | . | . | T | . | . | 0.12 | 0.69 | . | . | . | 0.00 | 0.21 |
| Gly | 228 | . | . | . | B | T | . | . | −0.56 | 1.01 | . | . | . | −0.20 | 0.21 |
| Leu | 229 | . | . | B | B | . | . | . | −0.94 | 0.94 | . | . | . | −0.60 | 0.27 |
| Gly | 230 | . | . | B | B | . | . | . | −1.00 | 0.99 | * | . | . | −0.60 | 0.20 |
| Tyr | 231 | . | . | B | B | . | . | . | −0.22 | 0.99 | * | . | . | −0.60 | 0.27 |
| Ile | 232 | . | . | B | B | . | . | . | 0.08 | 0.56 | * | . | . | −0.60 | 0.65 |
| Met | 233 | . | . | B | B | . | . | . | −0.39 | −0.13 | . | . | . | 0.45 | 1.09 |
| Ser | 234 | . | . | B | . | . | T | . | −0.43 | 0.13 | * | . | . | 0.25 | 0.57 |
| Arg | 235 | . | . | B | . | . | T | . | −0.30 | 0.01 | . | . | F | 0.55 | 0.61 |
| Asp | 236 | . | . | B | . | . | T | . | 0.06 | −0.24 | * | * | F | 1.30 | 0.95 |
| Leu | 237 | . | . | B | . | . | T | . | 0.06 | −0.86 | * | * | F | 1.90 | 1.39 |
| Val | 238 | . | . | B | B | . | . | . | 0.41 | −0.56 | * | . | F | 1.50 | 0.50 |
| Pro | 239 | . | . | B | B | . | . | . | 0.71 | 0.20 | * | . | . | 0.30 | 0.47 |
| Arg | 240 | . | . | B | B | . | . | . | 0.00 | 0.20 | * | . | . | 0.15 | 0.98 |
| Ile | 241 | A | . | . | B | . | . | . | −0.60 | 0.13 | * | * | . | 0.15 | 1.31 |
| Tyr | 242 | A | A | . | . | . | . | . | −0.13 | 0.10 | * | . | . | −0.15 | 0.84 |
| Glu | 243 | A | A | . | . | . | . | . | 0.69 | 0.10 | * | . | . | −0.30 | 0.42 |
| Met | 244 | A | A | . | . | . | . | . | 0.04 | 0.60 | * | * | . | −0.60 | 0.82 |
| Met | 245 | A | A | . | . | . | . | . | −0.02 | 0.56 | * | * | . | −0.60 | 0.39 |
| Gly | 246 | A | . | . | . | . | . | . | 0.66 | −0.20 | * | . | . | 0.50 | 0.45 |
| His | 247 | A | . | . | . | . | . | . | 0.01 | 0.23 | . | * | . | −0.10 | 0.70 |
| Val | 248 | A | . | . | . | . | . | . | 0.06 | 0.30 | . | * | . | −0.10 | 0.50 |
| Lys | 249 | A | . | . | . | . | . | . | −0.04 | −0.31 | . | * | F | 0.80 | 1.00 |
| Pro | 250 | A | . | . | . | . | . | . | 0.56 | 0.04 | . | . | F | 0.17 | 0.64 |
| Ile | 251 | A | . | . | . | . | . | . | 0.90 | −0.46 | . | . | F | 1.04 | 1.49 |
| Lys | 252 | A | . | . | . | . | . | . | 0.08 | −1.10 | . | . | F | 1.46 | 1.24 |
| Phe | 253 | . | . | B | B | . | . | . | 0.69 | −0.46 | . | . | F | 0.93 | 0.60 |
| Glu | 254 | . | . | B | B | . | . | . | −0.21 | −0.13 | . | . | F | 1.20 | 1.34 |
| Asp | 255 | . | . | B | B | . | . | . | −0.34 | −0.17 | . | * | . | 0.78 | 0.50 |
| Val | 256 | . | . | B | B | . | . | . | −0.34 | 0.26 | . | * | . | 0.06 | 0.57 |
| Tyr | 257 | . | . | B | B | . | . | . | −1.06 | 0.16 | . | * | . | −0.06 | 0.23 |
| Val | 258 | . | . | B | B | . | . | . | −1.17 | 0.73 | . | * | . | −0.48 | 0.07 |
| Gly | 259 | . | . | B | B | . | . | . | −1.17 | 1.41 | . | * | . | −0.60 | 0.08 |
| Ile | 260 | . | A | B | B | . | . | . | −1.98 | 1.17 | . | * | . | −0.60 | 0.08 |
| Cys | 261 | . | A | B | B | . | . | . | −1.93 | 1.10 | . | * | . | −0.60 | 0.09 |
| Leu | 262 | . | A | B | B | . | . | . | −1.64 | 1.14 | * | . | . | −0.60 | 0.08 |
| Asn | 263 | A | A | . | B | . | . | . | −1.64 | 0.71 | . | * | . | −0.60 | 0.22 |
| Leu | 264 | A | A | . | B | . | . | . | −1.30 | 0.67 | . | * | . | −0.60 | 0.31 |
| Leu | 265 | A | A | . | B | . | . | . | −1.30 | 0.50 | . | * | . | −0.60 | 0.60 |
| Lys | 266 | A | A | . | B | . | . | . | −0.67 | 0.50 | * | * | . | −0.60 | 0.26 |
| Val | 267 | . | A | B | B | . | . | . | −0.74 | 0.60 | . | * | . | −0.60 | 0.43 |
| Asn | 268 | . | A | B | B | . | . | . | −0.96 | 0.60 | * | * | . | −0.60 | 0.37 |
| Ile | 269 | . | A | B | B | . | . | . | −0.14 | 0.34 | * | * | . | −0.30 | 0.28 |
| His | 270 | . | A | B | B | . | . | . | 0.67 | 0.34 | . | * | . | −0.02 | 0.66 |
| Ile | 271 | . | . | B | B | . | . | . | 0.31 | −0.30 | . | * | . | 0.86 | 0.69 |
| Pro | 272 | . | . | B | B | . | . | . | 1.17 | −0.21 | . | * | F | 1.44 | 1.41 |
| Glu | 273 | . | . | . | . | T | . | . | 0.36 | −0.50 | . | * | F | 2.62 | 1.67 |
| Asp | 274 | . | . | . | . | T | T | . | 0.54 | −0.31 | . | * | F | 2.80 | 1.96 |
| Thr | 275 | A | . | . | . | . | T | . | −0.12 | −0.21 | . | . | F | 2.12 | 1.10 |
| Asn | 276 | A | . | . | . | . | T | . | −0.04 | 0.14 | . | . | F | 1.09 | 0.55 |
| Leu | 277 | A | . | . | . | . | T | . | −0.08 | 0.83 | . | * | . | 0.36 | 0.27 |
| Phe | 278 | A | . | . | B | . | . | . | 0.03 | 1.59 | . | * | . | −0.32 | 0.29 |
| Phe | 279 | A | . | . | B | . | . | . | −0.86 | 1.10 | . | * | . | −0.60 | 0.36 |
| Leu | 280 | A | . | . | B | . | . | . | −0.58 | 1.39 | . | * | . | 0.60 | 0.30 |
| Tyr | 281 | A | . | . | B | . | . | . | −1.39 | 1.20 | . | * | . | −0.60 | 0.48 |
| Arg | 282 | . | . | B | B | . | . | . | −0.58 | 1.10 | * | * | . | −0.60 | 0.46 |
| Ile | 283 | A | . | . | B | . | . | . | −0.73 | 0.31 | * | * | . | −0.30 | 0.92 |
| His | 284 | A | . | . | B | . | . | . | −0.70 | 0.27 | * | * | . | −0.30 | 0.44 |
| Leu | 285 | A | . | . | B | . | . | . | 0.11 | 0.09 | * | * | . | −0.30 | 0.12 |
| Asp | 286 | . | . | B | B | . | . | . | −0.46 | 0.49 | . | * | . | −0.60 | 0.30 |
| Val | 287 | . | . | B | B | . | . | . | −0.46 | 0.49 | * | * | . | −0.60 | 0.18 |
| Cys | 288 | . | . | B | B | . | . | . | 0.54 | −0.01 | * | . | . | 0.30 | 0.43 |
| Gln | 289 | A | . | . | B | . | . | . | −0.28 | −0.70 | * | . | . | 0.60 | 0.50 |
| Leu | 290 | A | . | . | B | . | . | . | −0.36 | −0.06 | * | . | . | 0.30 | 0.50 |
| Arg | 291 | A | . | . | B | . | . | . | −0.94 | −0.01 | * | . | . | 0.30 | 0.65 |
| Arg | 292 | . | . | B | B | . | . | . | −0.68 | −0.09 | * | . | . | 0.30 | 0.38 |
| Val | 293 | . | . | B | B | . | . | . | −0.04 | 0.01 | * | . | . | −0.30 | 0.47 |
| Ile | 294 | . | . | B | B | . | . | . | −0.39 | −0.17 | * | . | . | 0.30 | 0.32 |
| Ala | 295 | . | . | B | B | . | . | . | −0.28 | 0.26 | * | . | . | −0.30 | 0.16 |
| Ala | 296 | A | . | . | B | . | . | . | −0.69 | 1.04 | . | . | . | −0.60 | 0.19 |
| His | 297 | A | . | . | B | . | . | . | −1.10 | 0.79 | . | . | . | −0.39 | 0.37 |
| Gly | 298 | . | . | . | . | . | . | C | −0.20 | 0.49 | . | . | . | 0.22 | 0.48 |
| Phe | 299 | . | . | . | . | . | T | C | 0.69 | −0.01 | . | . | . | 1.53 | 0.96 |
| Ser | 300 | . | . | . | . | . | T | C | 0.39 | −0.51 | . | . | F | 2.34 | 1.22 |
| Ser | 301 | . | . | . | . | . | T | C | 0.09 | −0.33 | . | . | F | 2.10 | 0.87 |
| Lys | 302 | A | . | . | . | . | T | . | −0.19 | −0.07 | . | . | F | 1.69 | 0.70 |
| Glu | 303 | . | . | B | B | . | . | . | −0.54 | −0.37 | . | . | F | 1.08 | 0.75 |
| Ile | 304 | . | . | B | B | . | . | . | −0.13 | 0.03 | * | . | . | 0.12 | 0.49 |
| Ile | 305 | A | . | . | B | . | . | . | 0.17 | 0.56 | * | . | . | −0.39 | 0.26 |
| Thr | 306 | A | . | . | B | . | . | . | −0.39 | 0.96 | * | . | . | −0.60 | 0.26 |
| Phe | 307 | A | . | . | B | . | . | . | −1.03 | 1.60 | * | . | . | −0.60 | 0.27 |
| Trp | 308 | A | . | . | B | . | . | . | −1.84 | 1.53 | * | * | . | −0.60 | 0.38 |
| Gln | 309 | A | . | . | B | . | . | . | −0.84 | 1.53 | * | * | . | −0.60 | 0.22 |
| Val | 310 | . | . | B | B | . | . | . | 0.04 | 1.04 | * | * | . | −0.60 | 0.50 |
| Met | 311 | . | . | . | B | . | . | C | 0.04 | 0.66 | * | * | . | −0.40 | 0.76 |
| Leu | 312 | . | . | . | B | T | . | . | 0.43 | 0.23 | * | * | . | 0.10 | 0.63 |
| Arg | 313 | . | . | . | B | T | . | . | 0.06 | 0.31 | * | * | F | 0.40 | 1.23 |
| Asn | 314 | . | . | . | . | T | T | . | 0.02 | 0.24 | . | * | F | 0.65 | 0.66 |
| Thr | 315 | . | . | . | . | T | T | . | 0.63 | 0.13 | . | . | F | 0.80 | 1.10 |
| Thr | 316 | . | . | . | . | T | T | . | 0.84 | 0.20 | * | . | F | 0.65 | 0.88 |
| Cys | 317 | . | . | B | . | . | T | . | 1.27 | 0.63 | . | . | . | −0.20 | 0.70 |
| His | 318 | . | . | B | . | . | . | . | 0.77 | 0.66 | . | . | . | −0.40 | 0.62 |
| Tyr | 319 | . | . | B | . | . | . | . | 0.38 | 0.60 | . | . | . | −0.40 | 0.55 |

TABLE II

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | A | B | . | . | . | . | −0.83 | 0.16 | * | * | . | −0.30 | 0.32 |
| Leu | 2 | . | A | B | . | . | . | . | −1.11 | 0.42 | * | * | . | −0.60 | 0.21 |
| Cys | 3 | . | A | B | . | . | . | . | −1.01 | 0.56 | . | * | . | −0.60 | 0.09 |
| Arg | 4 | A | A | . | . | . | . | . | −1.43 | 1.04 | * | * | . | −0.60 | 0.09 |
| Leu | 5 | . | A | B | . | . | . | . | −1.90 | 1.11 | * | * | . | −0.60 | 0.09 |
| Cys | 6 | . | A | B | . | . | . | . | −1.60 | 1.07 | * | * | . | −0.60 | 0.13 |
| Trp | 7 | . | A | B | . | . | . | . | −1.03 | 0.89 | * | * | . | −0.60 | 0.09 |
| Leu | 8 | . | A | B | . | . | . | . | −0.67 | 1.64 | * | * | . | −0.60 | 0.17 |
| Val | 9 | . | A | B | . | . | . | . | −1.59 | 1.34 | * | * | . | −0.60 | 0.42 |
| Ser | 10 | . | A | B | . | . | . | . | −1.37 | 1.46 | . | * | . | −0.60 | 0.33 |
| Tyr | 11 | . | A | B | . | . | . | . | −1.56 | 1.04 | . | . | . | −0.60 | 0.40 |
| Ser | 12 | . | A | B | . | . | . | . | −2.08 | 1.00 | . | . | . | −0.60 | 0.40 |
| Leu | 13 | . | A | B | . | . | . | . | −2.08 | 1.04 | . | . | . | −0.60 | 0.25 |
| Ala | 14 | . | A | B | . | . | . | . | −2.03 | 1.34 | . | . | . | −0.60 | 0.13 |
| Val | 15 | . | A | B | . | . | . | . | −2.08 | 1.27 | . | . | . | −0.60 | 0.08 |
| Leu | 16 | . | A | B | . | . | . | . | −2.50 | 1.31 | . | . | . | −0.60 | 0.10 |
| Leu | 17 | . | A | B | . | . | . | . | −3.01 | 1.20 | . | . | . | −0.60 | 0.05 |
| Leu | 18 | A | A | . | . | . | . | . | −3.01 | 1.39 | . | . | . | −0.60 | 0.06 |
| Gly | 19 | A | A | . | . | . | . | . | −3.12 | 1.43 | . | . | . | −0.60 | 0.06 |
| Cys | 20 | A | A | . | . | . | . | . | −3.08 | 1.53 | . | * | . | −0.60 | 0.06 |
| Leu | 21 | A | A | . | . | . | . | . | −2.16 | 1.53 | * | . | . | −0.60 | 0.06 |
| Leu | 22 | A | A | . | . | . | . | . | −1.30 | 0.84 | * | . | . | −0.60 | 0.12 |
| Phe | 23 | A | A | . | . | . | . | . | −1.08 | 0.41 | . | . | . | −0.60 | −0.44 |
| Leu | 24 | A | A | . | . | . | . | . | −1.32 | 0.34 | * | . | . | −0.30 | 0.53 |
| Arg | 25 | A | A | . | . | . | . | . | −0.61 | 0.16 | * | * | . | −0.30 | 0.65 |
| Lys | 26 | A | A | . | . | . | . | . | −0.01 | −0.53 | * | . | F | 0.90 | 1.51 |
| Ala | 27 | A | A | . | . | . | . | . | 0.21 | −0.89 | * | * | F | 0.90 | 2.83 |
| Ala | 28 | . | A | . | . | . | . | C | 0.91 | −1.07 | * | . | F | 1.10 | 1.46 |
| Lys | 29 | . | A | . | . | . | . | C | 1.41 | −1.07 | * | * | F | 1.10 | 1.27 |
| Pro | 30 | . | A | . | . | . | . | C | 1.09 | −0.59 | * | * | F | 1.10 | 1.81 |
| Ala | 31 | . | A | . | . | T | . | . | 1.16 | −0.66 | . | * | F | 1.30 | 2.77 |
| Glu | 32 | . | A | B | . | . | . | . | 1.53 | −1.16 | . | * | F | 0.90 | 2.71 |
| Thr | 33 | . | . | B | . | . | T | . | 1.81 | −0.73 | . | * | F | 1.54 | 2.71 |
| Pro | 34 | . | . | B | . | . | T | . | 1.47 | −0.67 | . | . | F | 1.78 | 3.87 |
| Arg | 35 | . | . | B | . | . | T | . | 0.87 | −0.79 | . | . | F | 2.02 | 3.00 |
| Pro | 36 | . | . | B | . | . | T | . | 1.16 | −0.10 | . | . | F | 1.96 | 1.71 |
| Thr | 37 | . | . | . | . | T | . | . | 0.81 | −0.20 | . | . | F | 2.40 | 1.48 |
| Ser | 38 | . | . | B | . | . | . | . | 0.53 | −0.20 | . | . | F | 1.61 | 0.75 |
| Leu | 39 | . | . | B | . | . | T | . | 0.53 | −0.30 | . | . | F | 0.97 | 0.49 |
| Ser | 40 | . | . | . | . | T | T | . | 0.21 | −0.30 | . | . | F | 1.13 | 0.52 |
| Gly | 41 | . | . | . | . | . | T | C | 0.11 | −0.24 | . | . | F | 0.69 | 0.61 |
| Ala | 42 | . | . | . | . | . | T | C | 0.21 | −0.34 | . | * | F | 0.60 | 1.06 |
| Pro | 43 | . | . | . | . | . | T | C | 0.62 | −0.09 | * | . | F | 0.60 | 1.22 |
| Pro | 44 | . | . | . | . | . | T | C | 1.40 | −0.30 | * | * | F | 1.20 | 2.42 |
| Thr | 45 | . | . | B | . | . | T | . | 1.40 | −0.23 | * | . | F | 1.34 | 3.26 |
| Pro | 46 | . | . | B | . | . | T | . | 1.86 | −0.34 | * | . | F | 1.68 | 2.83 |
| Arg | 47 | . | . | . | . | T | . | . | 1.78 | −0.77 | * | . | F | 2.52 | 3.58 |
| His | 48 | . | . | B | . | . | T | . | 1.78 | −0.63 | * | . | F | 2.66 | 1.33 |
| Ser | 49 | . | . | . | . | T | T | . | 1.78 | −0.69 | * | . | F | 3.40 | 1.33 |
| Arg | 50 | . | . | . | . | T | T | . | 2.09 | −0.69 | * | . | F | 3.06 | 1.05 |
| Cys | 51 | . | . | B | . | . | T | . | 2.27 | −0.29 | * | . | F | 2.02 | 1.24 |
| Pro | 52 | . | . | . | . | . | T | C | 1.84 | −0.29 | * | * | F | 1.88 | 1.26 |
| Pro | 53 | . | . | . | . | T | T | . | 1.02 | −0.19 | . | * | F | 1.59 | 0.93 |
| Asn | 54 | . | . | . | . | T | T | . | 1.02 | 0.46 | . | * | F | 0.50 | 1.28 |
| His | 55 | . | . | B | . | . | T | . | 0.61 | 0.27 | . | * | . | 0.25 | 1.11 |
| Thr | 56 | . | . | B | . | . | . | . | 0.69 | 0.23 | . | . | . | −0.10 | 0.96 |
| Val | 57 | . | . | B | . | . | . | . | 0.60 | 0.30 | . | . | . | −0.10 | 0.61 |
| Ser | 58 | . | . | B | . | . | . | . | −0.00 | 0.29 | . | . | F | 0.05 | 0.60 |
| Ser | 59 | . | . | B | . | . | . | . | −0.30 | 0.47 | . | * | F | −0.25 | 0.34 |
| Ala | 60 | . | . | B | . | . | . | . | −1.08 | 0.37 | . | . | F | 0.05 | 0.62 |
| Ser | 61 | . | . | B | . | . | . | . | −0.98 | 0.41 | . | . | . | −0.40 | 0.38 |
| Leu | 62 | . | . | . | . | . | . | C | −0.42 | 0.46 | . | * | . | −0.20 | 0.44 |
| Ser | 63 | . | . | . | . | . | . | C | −0.01 | 0.46 | . | * | . | 0.00 | 0.58 |
| Leu | 64 | . | . | B | . | . | T | . | 0.26 | −0.04 | . | * | F | 1.25 | 0.85 |
| Pro | 65 | . | . | B | . | . | T | . | 0.96 | 0.07 | . | * | F | 1.00 | 1.40 |
| Ser | 66 | . | . | . | . | T | T | . | 0.44 | −0.61 | . | * | F | 2.50 | 2.04 |
| Arg | 67 | . | . | B | . | . | T | . | 0.56 | −0.31 | . | * | F | 2.00 | 2.04 |
| His | 68 | . | A | B | . | . | . | . | 0.04 | −0.21 | . | * | . | 1.25 | 1.15 |
| Arg | 69 | . | A | B | . | . | . | . | 0.54 | 0.04 | . | * | . | 0.30 | 0.70 |
| Leu | 70 | . | A | B | . | . | . | . | 0.51 | 0.14 | . | * | . | 0.10 | 0.52 |
| Phe | 71 | . | A | B | . | . | . | . | 0.92 | 0.90 | . | * | . | −0.40 | 0.60 |
| Leu | 72 | . | A | B | . | . | . | . | 0.78 | 0.40 | . | * | . | −0.60 | 0.60 |
| Thr | 73 | . | A | B | . | . | . | . | 0.14 | 0.90 | . | * | . | −0.60 | 0.99 |
| Tyr | 74 | . | . | B | B | . | . | . | 0.14 | 0.79 | * | * | . | −0.60 | 0.61 |
| Arg | 75 | . | . | . | B | T | . | . | 0.96 | 0.00 | * | . | . | 0.25 | 1.45 |
| His | 76 | . | . | . | B | T | . | . | 0.96 | −0.29 | * | . | . | 0.85 | 1.62 |
| Cys | 77 | . | . | . | . | T | T | . | 1.47 | 0.01 | * | * | . | 0.50 | 0.89 |
| Arg | 78 | . | . | . | . | T | T | . | 0.89 | −0.36 | * | . | . | 1.10 | −0.61 |
| Asn | 79 | . | . | . | . | T | T | . | 0.32 | 0.33 | * | . | . | 0.50 | 0.31 |
| Phe | 80 | . | . | B | . | . | T | . | −0.60 | 0.51 | * | . | . | −0.20 | 0.48 |
| Ser | 81 | . | A | B | . | . | . | . | −0.57 | 0.63 | * | * | . | −0.60 | 0.20 |
| Ile | 82 | . | A | B | . | . | . | . | −0.11 | 0.63 | * | * | . | −0.47 | 0.22 |
| Leu | 83 | . | A | B | . | . | . | . | −0.52 | 0.66 | * | * | . | −0.34 | 0.39 |
| Leu | 84 | . | A | B | . | . | . | . | −0.87 | 0.26 | . | * | . | 0.09 | 0.39 |
| Glu | 85 | . | A | B | . | . | . | . | −0.83 | 0.30 | . | * | F | 0.37 | 0.55 |
| Pro | 86 | . | . | . | . | T | T | . | −0.83 | 0.19 | . | * | F | 1.30 | 0.36 |
| Ser | 87 | . | . | . | . | T | T | . | 0.10 | −0.11 | . | * | F | 1.77 | 0.58 |
| Gly | 88 | . | . | . | . | T | T | . | 0.91 | −0.80 | . | . | F | 1.94 | 0.68 |
| Cys | 89 | . | . | . | . | T | T | . | 1.41 | −0.80 | . | * | F | 1.81 | 0.73 |
| Ser | 90 | . | . | . | . | T | T | . | 0.71 | −0.74 | . | . | F | 1.68 | 0.79 |
| Lys | 91 | A | . | . | . | . | T | . | 0.11 | −0.34 | . | . | F | 0.85 | 0.69 |
| Asp | 92 | A | . | . | . | . | T | . | −0.40 | −0.09 | . | . | F | 1.00 | 1.06 |
| Thr | 93 | A | . | . | . | . | T | . | −0.87 | 0.03 | . | . | F | 0.25 | 0.65 |
| Phe | 94 | A | A | . | . | . | . | . | −0.79 | 0.33 | . | . | . | −0.30 | 0.27 |
| Leu | 95 | A | A | . | . | . | . | . | −1.38 | 0.83 | . | * | . | −0.60 | 0.16 |
| Leu | 96 | A | A | . | . | . | . | . | −1.38 | 1.51 | . | * | . | −0.60 | 0.08 |
| Leu | 97 | A | A | . | . | . | . | . | −1.68 | 1.03 | . | * | . | −0.60 | 0.18 |
| Ala | 98 | A | A | . | . | . | . | . | −1.37 | 0.63 | . | * | . | −0.60 | 0.30 |
| Ile | 99 | A | A | . | . | . | . | . | −0.88 | 0.34 | . | * | . | −0.30 | 0.62 |
| Lys | 100 | A | A | . | . | . | . | . | −0.41 | 0.09 | . | * | F | 0.00 | 1.17 |
| Ser | 101 | . | A | . | . | . | . | C | 0.37 | −0.17 | . | . | F | 1.10 | 1.14 |
| Gln | 102 | . | . | . | . | . | T | C | 0.32 | −0.17 | . | * | F | 1.80 | 2.22 |
| Pro | 103 | . | . | . | . | . | T | C | 0.91 | −0.21 | * | . | F | 1.95 | 0.82 |
| Gly | 104 | . | . | . | . | . | T | C | 1.91 | −0.21 | * | * | F | 2.40 | 1.06 |
| His | 105 | . | . | . | . | . | T | C | 1.98 | −0.60 | * | * | F | 3.00 | 1.20 |
| Val | 106 | . | A | B | . | . | . | . | 1.69 | −1.00 | * | . | . | 1.95 | 1.52 |
| Glu | 107 | . | A | B | . | . | . | . | 1.10 | −0.93 | * | * | . | 1.65 | 1.55 |
| Arg | 108 | A | A | . | . | . | . | . | 0.42 | −0.86 | * | * | . | 1.35 | 1.15 |
| Arg | 109 | A | A | . | . | . | . | . | 0.88 | −0.67 | * | * | . | 1.05 | 1.09 |
| Ala | 110 | A | A | . | . | . | . | . | 0.61 | −1.31 | . | * | . | 0.75 | 1.23 |
| Ala | 111 | A | A | . | . | . | . | . | 1.16 | −0.93 | . | * | . | 0.60 | 0.84 |
| Ile | 112 | . | A | B | . | . | . | . | 0.87 | −0.44 | . | * | . | 0.30 | 0.62 |
| Arg | 113 | . | A | B | . | . | . | . | 0.41 | 0.47 | . | * | F | −0.45 | 0.65 |
| Ser | 114 | . | . | B | . | . | T | . | 0.41 | 0.40 | * | * | F | −0.05 | 0.63 |
| Thr | 115 | . | . | . | . | T | T | . | 0.71 | −0.10 | * | * | F | 1.40 | 1.77 |
| Trp | 116 | . | . | . | . | T | T | . | 0.96 | 0.13 | * | . | F | 0.65 | 0.95 |
| Gly | 117 | . | . | . | . | . | T | C | 1.84 | 0.56 | * | . | F | 0.40 | 0.70 |
| Arg | 118 | . | . | . | . | T | . | . | 1.39 | 0.17 | * | . | F | 0.95 | 0.81 |
| Trp | 119 | . | . | . | . | T | T | . | 0.88 | 0.11 | * | . | F | 1.40 | 0.76 |
| Gly | 120 | . | . | . | . | . | T | C | 0.84 | −0.11 | * | . | F | 2.05 | 0.64 |
| Asp | 121 | . | . | . | . | T | T | . | 0.92 | −0.11 | * | . | F | 2.50 | 0.32 |
| Gly | 122 | . | . | . | . | . | T | C | 0.68 | 0.31 | * | . | F | 1.45 | 0.47 |
| Leu | 123 | . | . | . | . | . | . | C | −0.24 | −0.10 | * | . | F | 1.60 | 0.48 |
| Gly | 124 | . | . | . | . | . | T | C | 0.09 | 0.16 | * | * | F | 0.95 | 0.24 |
| Pro | 125 | . | . | . | . | . | T | C | −0.38 | 0.16 | * | . | F | 0.70 | 0.48 |
| Ala | 126 | A | . | . | . | . | T | . | −1.23 | 0.41 | * | * | F | −0.05 | 0.48 |
| Leu | 127 | A | . | . | . | . | T | . | −1.59 | 0.37 | . | . | . | 0.10 | 0.36 |
| Lys | 128 | . | A | B | . | . | . | . | −1.59 | 0.73 | * | * | . | −0.60 | 0.20 |
| Leu | 129 | . | A | B | . | . | . | . | −2.06 | 0.99 | . | . | . | −0.60 | 0.17 |
| Val | 130 | . | A | B | . | . | . | . | −2.19 | 1.17 | . | * | . | −0.60 | 0.17 |
| Phe | 131 | . | A | B | . | . | . | . | −2.46 | 0.91 | . | * | . | −0.60 | 0.08 |
| Leu | 132 | . | A | B | . | . | . | . | −2.23 | 1.56 | . | * | . | −0.60 | 0.07 |
| Leu | 133 | . | A | B | . | . | . | . | −2.62 | 1.37 | . | * | . | −0.60 | 0.10 |
| Gly | 134 | . | . | B | B | . | . | . | −2.11 | 1.16 | . | . | . | −0.60 | 0.11 |
| Val | 135 | . | . | B | B | . | . | . | −1.84 | 0.76 | . | . | . | −0.60 | 0.19 |
| Ala | 136 | . | . | . | B | . | . | C | −1.36 | 0.57 | . | . | . | −0.40 | 0.23 |
| Gly | 137 | . | . | . | B | . | . | C | −0.76 | 0.31 | . | . | F | 0.05 | 0.36 |
| Ser | 138 | . | . | . | . | . | . | C | −0.53 | 0.31 | . | . | F | 0.25 | 0.74 |
| Ala | 139 | . | . | . | . | . | . | C | −0.19 | 0.17 | . | . | F | 0.25 | 0.74 |
| Pro | 140 | . | . | . | . | . | T | C | −0.14 | 0.07 | . | . | F | 0.60 | 1.30 |
| Pro | 141 | A | . | . | . | . | T | . | −0.37 | 0.33 | . | . | F | 0.25 | 0.80 |
| Ala | 142 | A | . | . | . | . | T | . | −0.61 | 0.63 | . | . | F | −0.05 | 0.65 |
| Gin | 143 | A | . | . | . | . | T | . | −0.56 | 0.63 | . | . | . | −0.20 | 0.43 |
| Leu | 144 | . | A | B | . | . | . | . | 0.03 | 0.96 | . | . | . | −0.60 | 0.43 |
| Leu | 145 | A | A | . | . | . | . | . | −0.06 | 0.53 | * | * | . | −0.60 | 0.74 |
| Ala | 146 | A | A | . | . | . | . | . | 0.27 | 0.41 | * | . | . | −0.60 | 0.57 |
| Tyr | 147 | A | A | . | . | . | . | . | 0.86 | 0.01 | * | . | . | −0.15 | 1.36 |
| Glu | 148 | A | A | . | . | . | . | . | 0.16 | −0.67 | * | . | F | 0.90 | 2.86 |
| Ser | 149 | A | A | . | . | . | . | . | 0.97 | −0.57 | * | . | F | 0.90 | 2.45 |
| Arg | 150 | A | A | . | . | . | . | . | 1.78 | −1.07 | * | . | F | 0.90 | 2.61 |
| Gln | 151 | A | A | . | . | . | . | . | 1.48 | −1.83 | * | . | F | 0.90 | 2.52 |
| Phe | 152 | A | A | . | . | . | . | . | 0.91 | −1.14 | * | . | F | 0.90 | 1.32 |
| Asp | 153 | A | A | . | . | . | . | . | 0.91 | −0.84 | * | . | F | 0.75 | 0.55 |
| Asp | 154 | A | A | . | . | . | . | . | 0.92 | −0.44 | * | * | F | 0.45 | 0.55 |
| Ile | 155 | A | A | . | . | . | . | . | 0.81 | 0.47 | * | * | . | −0.60 | 0.67 |
| Leu | 156 | A | A | . | . | . | . | . | 0.11 | −0.31 | * | * | . | 0.30 | 0.67 |
| Gln | 157 | A | A | . | . | . | . | . | 0.50 | 0.47 | * | * | . | −0.60 | 0.35 |
| Trp | 158 | A | A | . | . | . | . | . | 0.50 | 0.96 | . | * | . | −0.60 | 0.72 |
| Asp | 159 | A | A | . | . | . | . | . | 0.50 | 0.27 | . | * | . | −0.15 | 1.51 |
| Phe | 160 | A | A | . | . | . | . | . | 0.69 | −0.41 | . | * | . | 0.45 | 1.45 |
| Thr | 161 | A | A | . | . | . | . | . | 0.80 | −0.03 | * | * | F | 0.60 | 1.20 |
| Glu | 162 | A | A | . | . | . | . | . | 0.80 | −0.16 | * | * | F | 0.45 | 0.62 |
| Asp | 163 | A | A | . | . | . | . | . | 0.28 | 0.24 | * | * | F | 0.00 | 1.15 |
| Phe | 164 | A | A | . | . | . | . | . | −0.03 | 0.14 | * | * | . | −0.30 | 0.66 |
| Phe | 165 | A | A | . | . | . | . | . | −0.14 | 0.14 | * | * | . | −0.30 | 0.55 |
| Asn | 166 | A | A | . | . | . | . | . | 0.21 | 0.83 | * | * | . | −0.60 | 0.27 |
| Leu | 167 | A | A | . | . | . | . | . | 0.22 | 0.83 | * | * | . | −0.60 | 0.63 |
| Thr | 168 | A | A | . | . | . | . | . | −0.60 | 0.04 | * | . | . | −0.15 | 1.25 |
| Leu | 169 | A | A | . | . | . | . | . | 0.07 | −0.06 | * | * | . | 0.30 | 0.64 |
| Lys | 170 | A | A | . | . | . | . | . | −0.04 | 0.04 | . | * | . | −0.15 | 1.06 |
| Glu | 171 | A | A | . | . | . | . | . | −0.04 | 0.04 | . | . | . | −0.30 | 0.61 |
| Leu | 172 | A | A | . | . | . | . | . | 0.88 | −0.04 | . | . | . | 0.45 | 1.27 |
| His | 173 | A | A | . | . | . | . | . | 0.90 | −0.73 | . | . | . | 0.75 | 1.25 |
| Leu | 174 | A | A | . | . | . | . | . | 0.86 | 0.19 | . | . | . | −0.30 | 0.76 |
| Gln | 175 | A | A | . | . | . | . | . | −0.04 | 0.83 | . | . | . | −0.60 | 0.68 |
| Arg | 176 | A | A | . | . | . | . | . | −0.63 | 0.79 | . | . | . | −0.60 | 0.37 |
| Trp | 177 | A | A | . | . | . | . | . | −0.41 | 0.79 | * | . | . | −0.60 | 0.46 |
| Val | 178 | . | A | B | . | . | . | . | −1.04 | 0.60 | * | . | . | −0.60 | 0.27 |
| Val | 179 | . | A | B | . | . | . | . | −0.44 | 0.77 | * | . | . | −0.60 | 0.07 |
| Ala | 180 | . | A | B | . | . | . | . | −0.44 | 1.20 | * | * | . | −0.60 | 0.11 |
| Ala | 181 | . | A | B | . | . | . | . | −1.14 | 0.69 | * | * | . | −0.60 | 0.25 |
| Cys | 182 | A | . | . | . | . | T | . | −0.89 | 0.54 | . | * | . | −0.20 | 0.34 |
| Pro | 183 | A | . | . | . | . | T | . | −0.73 | 0.40 | . | . | . | −0.20 | 0.46 |
| Gin | 184 | A | . | . | . | . | T | . | −0.48 | 0.69 | . | * | . | −0.20 | 0.39 |
| Ala | 185 | A | . | . | . | . | T | . | −0.70 | 0.80 | . | * | . | −0.20 | 0.72 |
| His | 186 | A | A | . | . | . | . | . | −0.07 | 0.91 | . | * | . | −0.60 | 0.39 |
| Phe | 187 | A | A | . | . | . | . | . | 0.26 | 0.49 | . | * | . | −0.26 | 0.45 |
| Net | 188 | . | A | B | . | . | . | . | 0.47 | 0.51 | . | * | . | 0.08 | −0.44 |
| Leu | 189 | . | A | B | . | . | . | . | 0.47 | 0.01 | . | * | . | 0.72 | 0.54 |
| Lys | 190 | . | A | . | . | T | . | . | 1.06 | −0.49 | * | * | F | 2.36 | 1.03 |
| Gly | 191 | . | . | . | . | T | T | . | 0.23 | −1.27 | * | * | F | 3.40 | 1.74 |
| Asp | 192 | A | . | . | . | . | T | . | 0.23 | −1.24 | . | * | F | 2.66 | 1.57 |
| Asp | 193 | A | . | . | . | . | T | . | −0.02 | −1.14 | * | * | F | 2.17 | 0.68 |
| Asp | 194 | A | . | . | . | . | T | . | 0.76 | −0.50 | * | * | F | 1.53 | 0.51 |
| Val | 195 | . | . | B | B | . | . | . | −0.14 | −0.43 | * | * | . | 0.64 | 0.41 |
| Phe | 196 | . | . | B | B | . | . | . | −0.01 | 0.21 | . | * | . | −0.30 | 0.18 |
| Val | 197 | . | . | B | B | . | . | . | −0.01 | 0.64 | . | . | . | −0.60 | 0.17 |
| His | 198 | . | . | B | B | . | . | . | −0.87 | 1.04 | * | . | . | −0.60 | 0.37 |
| Val | 199 | . | . | B | B | . | . | . | −1.68 | 1.04 | * | . | . | −0.60 | 0.32 |
| Pro | 200 | . | . | . | B | . | . | C | −0.82 | 0.94 | * | * | . | −0.40 | 0.35 |
| Asn | 201 | . | A | . | B | . | . | C | −0.82 | 0.30 | * | . | . | −0.10 | 0.45 |
| Val | 202 | A | A | . | B | . | . | . | −0.78 | 0.59 | * | . | . | −0.60 | 0.52 |
| Leu | 203 | . | A | B | B | . | . | . | −0.74 | 0.63 | * | . | . | −0.60 | 0.28 |
| Glu | 204 | . | A | B | B | . | . | . | −0.23 | 0.20 | * | . | . | −0.30 | 0.29 |
| Phe | 205 | . | A | B | . | . | . | . | −0.31 | 0.23 | * | . | . | −0.06 | 0.39 |
| Leu | 206 | . | . | B | . | . | T | . | −0.31 | 0.50 | * | . | . | 0.28 | 0.49 |
| Asp | 207 | . | . | . | . | T | T | . | 0.33 | −0.19 | * | . | F | 1.97 | 0.48 |
| Gly | 208 | . | . | . | . | T | T | . | 0.56 | 0.24 | . | . | F | 1.61 | 0.85 |
| Trp | 209 | . | . | . | . | . | T | C | 0.56 | −0.04 | * | . | F | 2.40 | 1.04 |
| Asp | 210 | . | . | . | . | . | T | C | 1.26 | −0.33 | * | . | F | 2.16 | 1.08 |
| Pro | 211 | A | . | . | . | . | T | . | 1.26 | −0.33 | * | . | F | 1.72 | 1.82 |
| Ala | 212 | A | . | . | . | . | T | . | 0.44 | −0.07 | * | . | F | 1.48 | 1.43 |
| Gln | 213 | A | . | . | . | . | T | . | −0.07 | −0.30 | * | . | F | 1.09 | 0.70 |
| Asp | 214 | . | A | B | B | . | . | . | −0.12 | 0.34 | . | . | F | −0.15 | 0.34 |
| Leu | 215 | . | A | B | B | . | . | . | −0.12 | 0.34 | . | . | . | −0.30 | 0.33 |
| Leu | 216 | . | A | B | B | . | . | . | −0.77 | −0.16 | . | . | . | 0.30 | 0.32 |
| Val | 217 | . | A | B | B | . | . | . | −1.07 | 0.09 | * | . | . | −0.30 | 0.14 |
| Gly | 218 | . | . | B | B | . | . | . | −0.96 | 0.77 | * | . | . | −0.60 | 0.12 |
| Asp | 219 | . | . | B | B | . | . | . | −0.96 | 0.09 | * | . | . | −0.30 | 0.29 |
| Val | 220 | . | . | B | B | . | . | . | −0.73 | −0.20 | * | . | . | 0.30 | 0.67 |
| Ile | 221 | . | . | B | B | . | . | . | −0.73 | −0.34 | * | . | . | 0.30 | 0.68 |
| Arg | 222 | . | . | B | B | . | . | . | −0.09 | −0.09 | * | . | . | 0.30 | 0.34 |
| Gln | 223 | . | . | B | B | . | . | . | 0.26 | 0.34 | . | * | . | −0.30 | 0.70 |
| Ala | 224 | . | . | B | B | . | . | . | 0.37 | 0.10 | * | . | . | 0.19 | 1.6 |
| Leu | 225 | . | . | . | . | . | T | C | 1.22 | −0.59 | . | * | F | 2.18 | 1.61 |
| Pro | 226 | . | . | . | . | . | T | C | 1.80 | −0.19 | . | * | F | 2.22 | 1.50 |
| Asn | 227 | . | . | . | . | T | T | . | 1.73 | −0.10 | . | * | F | 2.76 | 2.14 |
| Arg | 228 | . | . | . | . | T | T | . | 0.88 | −0.60 | . | * | F | 3.40 | 5.18 |
| Asn | 229 | . | . | . | B | T | . | . | 1.51 | −0.64 | . | * | F | 2.66 | 2.49 |
| Thr | 230 | . | . | . | B | T | . | . | 2.08 | −1.07 | . | . | F | 2.32 | 3.09 |
| Lys | 231 | . | . | B | B | . | . | . | 1.59 | −0.71 | . | * | F | 1.58 | 2.47 |
| Val | 232 | . | . | B | B | . | . | . | 0.70 | 0.07 | . | * | F | 0.34 | 1.33 |
| Lys | 233 | . | . | B | B | . | . | . | 0.38 | 0.36 | . | * | . | −0.30 | 0.65 |
| Tyr | 234 | . | . | B | B | . | . | . | 0.17 | 0.30 | . | * | . | −0.30 | 0.50 |
| Phe | 235 | . | . | B | B | . | . | . | 0.18 | 0.73 | . | * | . | −0.45 | 1.04 |
| Ile | 236 | . | . | B | B | . | . | . | −0.47 | 0.47 | . | * | . | −0.60 | 0.70 |
| Pro | 237 | . | . | B | . | . | T | . | 0.14 | 1.09 | . | * | . | −0.20 | 0.44 |
| Pro | 238 | . | . | . | . | T | T | . | 0.21 | 1.09 | . | * | . | 0.20 | 0.80 |
| Ser | 239 | . | . | . | . | T | T | . | −0.13 | 0.30 | * | . | . | 0.65 | 2.23 |
| Met | 240 | . | . | B | . | . | T | . | 0.26 | 0.11 | * | . | . | 0.25 | 1.46 |
| Tyr | 241 | . | . | B | . | . | . | . | 1.11 | 0.17 | * | . | . | 0.05 | 1.36 |
| Arg | 242 | . | . | B | . | . | . | . | 1.08 | 0.24 | * | . | . | 0.05 | 1.38 |
| Ala | 243 | . | . | B | . | . | . | . | 1.08 | 0.61 | * | . | . | −0.25 | 2.19 |
| Thr | 244 | . | . | B | . | . | . | . | 1.17 | 0.43 | * | . | . | −0.25 | 2.16 |
| His | 245 | . | . | B | . | . | . | . | 1.52 | 0.10 | * | . | . | 0.05 | 1.70 |
| Tyr | 246 | . | . | B | . | . | . | . | 1.18 | 0.86 | * | * | . | −0.25 | 2.64 |
| Pro | 247 | . | . | B | . | . | T | . | 0.72 | 0.86 | . | * | F | 0.10 | 1.85 |
| Pro | 248 | . | . | B | . | . | T | . | 0.97 | 0.80 | . | . | F | 0.10 | 1.35 |
| Tyr | 249 | . | . | . | . | T | T | . | 0.93 | 0.73 | . | . | F | 0.35 | 0.85 |
| Ala | 250 | . | . | . | . | T | T | . | 0.62 | 0.40 | . | . | F | 0.35 | 0.54 |
| Gly | 251 | . | . | . | . | T | T | . | 0.62 | 0.40 | . | . | F | 0.35 | 0.35 |
| Gly | 252 | . | . | . | . | T | T | . | −0.02 | 0.73 | . | . | F | 0.35 | 0.35 |
| Gly | 253 | . | . | B | . | . | T | . | −0.41 | 0.61 | . | . | F | −0.05 | 0.26 |
| Gly | 254 | . | . | B | . | . | T | . | −0.47 | 0.73 | * | * | F | −0.05 | 0.26 |
| Tyr | 255 | . | . | B | B | . | . | . | 0.23 | 0.69 | * | * | . | −0.60 | 0.35 |
| Val | 256 | . | . | B | B | . | . | . | −0.01 | 0.26 | . | * | . | −0.30 | 0.69 |
| Met | 257 | . | . | B | B | . | . | . | 0.02 | 0.33 | . | * | . | −0.30 | 0.70 |
| Ser | 258 | . | . | B | B | . | . | . | −0.49 | 0.39 | * | * | . | −0.30 | 0.64 |
| Arg | 259 | . | . | B | B | . | . | . | −0.03 | 0.27 | * | * | . | −0.30 | 0.64 |
| Ala | 260 | . | . | B | B | . | . | . | 0.32 | −0.37 | * | * | . | 0.45 | 1.28 |
| Thr | 261 | A | . | . | B | . | . | . | 0.37 | −0.99 | * | * | . | 0.75 | 1.86 |
| Val | 262 | A | A | . | . | . | . | . | 0.97 | −0.59 | * | * | . | 0.60 | 0.78 |
| Arg | 263 | A | A | . | . | . | . | . | 0.68 | −0.29 | * | * | . | 0.45 | 1.35 |
| Arg | 264 | A | A | . | . | . | . | . | −0.32 | −0.29 | * | * | . | 0.30 | 0.94 |
| Leu | 265 | A | A | . | . | . | . | . | −0.33 | −0.09 | * | . | . | 0.30 | 0.89 |
| Gin | 266 | A | A | . | . | . | . | . | −0.02 | −0.11 | * | . | . | 0.30 | 0.45 |
| Ala | 267 | A | A | . | . | . | . | . | 0.83 | −0.11 | * | . | . | 0.30 | 0.40 |
| Ile | 268 | A | A | . | . | . | . | . | 0.13 | −0.11 | * | . | . | 0.30 | 0.80 |
| Met | 269 | A | A | . | . | . | . | . | 0.02 | −0.30 | . | * | . | 0.30 | 0.47 |
| Glu | 270 | A | A | . | . | . | . | . | 0.02 | −0.70 | * | . | . | 0.60 | 0.80 |
| Asp | 271 | A | A | . | . | . | . | . | −0.68 | −0.51 | * | . | F | 0.75 | 0.95 |
| Ala | 272 | A | A | . | . | . | . | . | −0.30 | −0.41 | . | . | . | 0.30 | 0.83 |
| Glu | 273 | A | A | . | . | . | . | . | −0.30 | −0.60 | . | . | . | 0.60 | 0.74 |
| Leu | 274 | A | A | . | . | . | . | . | 0.30 | 0.09 | . | . | . | −0.30 | 0.31 |
| Phe | 275 | A | A | . | . | . | . | . | 0.30 | 0.09 | . | . | . | −0.30 | 0.51 |
| Pro | 276 | A | A | . | . | . | . | . | −0.56 | −0.41 | . | . | . | 0.30 | 0.49 |
| Ile | 277 | A | . | . | B | . | . | . | −0.67 | 0.23 | . | . | . | −0.30 | 0.45 |
| Asp | 278 | A | . | . | B | . | . | . | −1.52 | 0.33 | . | . | F | −0.15 | 0.45 |
| Asp | 279 | A | . | . | B | . | . | . | −1.06 | 0.19 | . | . | F | −0.15 | 0.21 |
| Val | 280 | A | . | . | B | . | . | . | −0.96 | 0.19 | . | . | . | −0.30 | 0.30 |
| Phe | 281 | A | . | . | B | . | . | . | −1.41 | 0.11 | . | . | . | −0.30 | 0.18 |
| Val | 282 | . | . | B | B | . | . | . | −1.33 | 0.69 | . | * | . | −0.60 | 0.06 |
| Gly | 283 | . | . | B | B | . | . | . | −1.22 | 1.37 | . | . | . | −0.60 | 0.06 |
| Met | 284 | . | . | B | B | . | . | . | −1.11 | 0.73 | * | * | . | −0.60 | 0.14 |
| Cys | 285 | . | . | B | B | . | . | . | −1.07 | −0.06 | * | . | . | 0.30 | 0.38 |
| Leu | 286 | . | . | B | B | . | . | . | −0.71 | −0.01 | * | . | . | 0.30 | 0.32 |
| Arg | 287 | . | . | B | B | . | . | . | −0.67 | −0.01 | * | . | . | 0.30 | 0.32 |
| Arg | 288 | A | . | . | B | . | . | . | −0.62 | 0.06 | * | . | . | −0.30 | 0.49 |
| Leu | 289 | . | . | . | B | T | . | . | −0.23 | −0.13 | * | . | . | 0.70 | 0.79 |
| Gly | 290 | . | . | . | . | T | . | . | −0.17 | −0.39 | * | . | . | 0.90 | 0.63 |
| Leu | 291 | . | . | . | . | . | . | C | 0.61 | 0.23 | * | . | . | 0.10 | 0.32 |
| Ser | 292 | . | . | . | . | . | . | C | 0.47 | 0.73 | * | . | . | −0.20 | 0.52 |
| Pro | 293 | . | . | B | . | . | . | . | −0.23 | 0.54 | * | * | . | −0.40 | 0.72 |
| Met | 294 | . | . | B | . | . | . | . | 0.23 | 0.61 | . | . | . | −0.40 | 0.88 |
| His | 295 | A | . | . | . | . | . | . | −0.12 | 0.36 | * | * | . | −0.10 | 0.65 |
| His | 296 | A | . | . | . | . | . | . | 0.73 | 0.76 | * | * | . | −0.40 | 0.36 |
| Ala | 297 | A | . | . | . | . | . | . | 0.72 | 0.33 | * | . | . | −0.10 | 0.73 |
| Gly | 298 | A | . | . | B | . | . | . | 0.23 | 0.20 | . | . | . | −0.30 | 0.78 |
| Phe | 299 | A | . | . | B | . | . | . | 0.49 | 0.49 | . | . | . | −0.60 | 0.50 |
| Lys | 300 | A | . | . | B | . | . | . | −0.37 | 0.41 | . | * | . | −0.60 | 0.49 |
| Thr | 301 | . | . | B | B | . | . | . | −0.22 | 0.60 | . | . | . | −0.60 | 0.34 |
| Phe | 302 | . | . | B | B | . | . | . | 0.48 | 0.17 | . | . | . | −0.30 | 0.78 |
| Gly | 303 | . | . | B | B | . | . | . | 0.61 | −0.61 | . | . | . | 0.90 | 0.76 |
| Ile | 304 | . | . | B | B | . | . | . | 0.50 | −0.19 | . | * | . | 0.90 | 0.82 |
| Arg | 305 | . | . | B | B | . | . | . | 0.46 | 0.01 | * | * | F | 0.75 | 0.78 |
| Arg | 306 | . | . | B | . | . | . | . | 0.56 | −0.77 | * | * | F | 2.30 | 1.31 |
| Pro | 307 | . | . | . | . | T | . | . | 0.44 | −0.77 | * | * | F | 3.00 | 2.90 |
| Leu | 308 | . | . | . | . | T | . | . | 0.79 | −0.77 | * | . | F | 2.70 | 1.22 |
| Asp | 309 | . | . | . | . | . | . | C | 1.47 | −0.77 | * | . | F | 2.20 | 1.04 |
| Pro | 310 | . | . | . | . | T | . | . | 0.69 | −0.34 | * | . | F | 1.80 | 1.04 |
| Leu | 311 | . | . | . | . | T | . | . | −0.23 | −0.20 | * | . | F | 1.35 | 0.68 |
| Asp | 312 | . | . | B | . | . | T | . | −0.27 | −0.20 | * | . | F | 0.85 | 0.33 |
| Pro | 313 | . | . | B | . | . | T | . | 0.66 | 0.56 | * | . | F | −0.05 | 0.34 |
| Cys | 314 | . | . | B | . | . | T | . | 0.31 | 0.13 | * | . | . | 0.10 | 0.80 |
| Leu | 315 | . | . | B | . | . | T | . | −0.29 | −0.13 | * | . | . | 0.70 | 0.48 |
| Tyr | 316 | . | . | B | B | . | . | . | −0.29 | 0.56 | * | . | . | −0.60 | 0.25 |
| Arg | 317 | . | . | B | B | . | . | . | −1.10 | 0.81 | * | . | . | −0.60 | 0.39 |
| Gly | 318 | . | . | B | B | . | . | . | −1.74 | 0.93 | * | . | . | −0.60 | 0.39 |
| Leu | 319 | . | . | B | B | . | . | . | −1.11 | 0.89 | * | . | . | −0.60 | 0.18 |
| Leu | 320 | . | . | B | B | . | . | . | −0.19 | 0.63 | * | * | . | −0.60 | 0.13 |
| Leu | 321 | . | . | B | B | . | . | . | −0.76 | 0.63 | * | * | . | −0.60 | 0.25 |
| Val | 322 | . | . | B | B | . | . | . | −1.17 | 0.89 | * | * | . | −0.60 | 0.25 |
| His | 323 | . | . | B | B | . | . | . | −1.03 | 0.59 | * | . | . | −0.42 | 0.41 |
| Arg | 324 | . | . | B | B | . | . | . | −1.03 | 0.33 | . | . | . | 0.06 | 0.77 |
| Leu | 325 | . | . | . | B | . | . | C | −0.22 | 0.33 | . | . | . | 0.44 | 0.86 |
| Ser | 326 | . | . | . | . | . | T | C | −0.01 | −0.31 | . | . | . | 1.77 | 1.10 |
| Pro | 327 | . | . | . | . | . | T | C | 0.56 | −0.20 | . | . | . | 1.80 | 0.55 |
| Leu | 328 | A | . | . | . | . | T | . | 0.28 | 0.71 | . | . | . | 0.52 | 0.71 |
| Glu | 329 | A | . | . | . | . | T | . | −0.43 | 0.51 | . | * | . | 0.34 | 0.76 |
| Met | 330 | A | . | . | B | . | . | . | 0.09 | 0.74 | . | . | . | −0.24 | 0.49 |
| Trp | 331 | A | . | . | B | . | . | . | −0.20 | 1.23 | . | . | . | −0.42 | 0.62 |
| Thr | 332 | A | . | . | B | . | . | . | −0.80 | 1.04 | . | . | . | −0.60 | 0.36 |
| Met | 333 | A | . | . | B | . | . | . | −0.84 | 1.73 | . | . | . | −0.60 | 0.30 |
| Trp | 334 | A | . | . | B | . | . | . | −1.16 | 1.76 | . | . | . | −0.60 | 0.21 |
| Ala | 335 | A | . | . | B | . | . | . | −0.56 | 1.33 | . | . | . | −0.60 | 0.21 |
| Leu | 336 | A | . | . | B | . | . | . | −0.27 | 0.84 | . | . | . | −0.60 | 0.36 |
| Val | 337 | A | . | . | B | . | . | . | −0.30 | 0.23 | . | . | . | −0.30 | 0.59 |
| Thr | 338 | A | . | . | . | . | T | . | −0.51 | −0.26 | * | . | F | 0.85 | 0.58 |
| Asp | 339 | A | . | . | . | . | T | . | −0.18 | −0.07 | * | . | F | 0.85 | 0.58 |
| Glu | 340 | A | . | . | . | . | T | . | −0.26 | −0.76 | * | . | F | 1.30 | 1.56 |
| Gly | 341 | A | . | . | . | . | T | . | −0.03 | −0.83 | . | . | F | 1.15 | 0.58 |
| Leu | 342 | A | A | . | . | . | . | . | 0.23 | −0.81 | * | . | F | 0.75 | 0.35 |
| Lys | 343 | A | A | . | . | . | . | . | 0.20 | −0.31 | * | . | . | 0.30 | 0.20 |
| Cys | 344 | . | A | B | . | . | . | . | −0.01 | 0.11 | * | . | . | −0.30 | 0.20 |
| Ala | 345 | . | A | . | . | T | . | . | −0.90 | 0.11 | * | . | . | 0.10 | 0.38 |
| Ala | 346 | . | A | B | . | . | . | . | −0.77 | 0.11 | * | . | . | −0.30 | 0.13 |
| Gly | 347 | . | A | B | . | . | . | . | 0.04 | 0.54 | * | * | F | −0.45 | 0.39 |
| Pro | 348 | . | . | B | . | . | . | . | 0.11 | 0.37 | . | * | F | 0.05 | 0.67 |
| Ile | 349 | . | . | B | . | . | . | . | 0.39 | −0.13 | . | * | F | 0.80 | 1.29 |
| Pro | 350 | . | . | B | . | . | . | . | 0.59 | −0.20 | . | * | . | 0.65 | 1.67 |
| Gln | 351 | . | . | B | . | . | . | . | 0.79 | −0.20 | * | * | . | 0.65 | 1.38 |
| Arg | 352 | . | . | B | . | . | . | . | 0.74 | −0.20 | . | * | . | 0.65 | 2.51 |

Among highly preferred fragments in this regard are those that comprise regions of Dendriac or Brainiac-3 that combine several structural features, such as several of the features set out above.

In another embodiment, the invention provides an isolated nucleic acid molecule comprising a polynucleotide which hybridizes under stringent hybridization conditions to a portion of the polynucleotide in a nucleic acid molecule of the invention described above, for instance, the Dendriac cDNA clone contained in ATCC Deposit Nos. 203056 and 209627 or the Brainiac-3 cDNA clone contained in ATCC Deposit Nos. 203451 and 209463. By “stringent hybridization conditions” is intended overnight incubation at 42° C. in a solution comprising: 50% formamide, 5× SSC (750 mM NaCl, 75 mM trisodium citrate), 50 mM sodium phosphate (pH 7.6), 5× Denhardts solution, 10% dextran sulfate, and 20 μg/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNAs or the nucleotide sequence as shown in FIGS. 1A, 1B, and 1C (SEQ ID NO:1) and SEQ ID NO:9, and FIGS. 2A and 2B (SEQ ID NO:3) and SEQ ID NO:11). Of course, a polynucleotide which hybridizes only to a poly A sequence (such as the 3′ terminal poly(A) tract of the Dendriac or Brainiac-3 cDNAs shown in FIGS. 1A, 1B, and 1C (SEQ ID NO:1) and SEQ ID NO:9, and FIGS. 2A and 2B (SEQ ID NO:3) and SEQ ID NO:11, respectively), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a Dendriac polypeptide may include, but are not limited to those encoding the amino acid sequence of the mature polypeptide, by itself; and the coding sequence for the mature polypeptide and additional sequences, such as those encoding the about 25 amino acid residue leader or secretory sequence, such as a pre-, or pro- or prepro-protein sequence; the coding sequence of the mature polypeptide, with or without the aforementioned additional coding sequences.

Also as indicated, nucleic acid molecules of the present invention which encode a Brainiac-3 polypeptide may include, but are not limited to those encoding the amino acid sequence of the mature polypeptide, by itself; and the coding sequence for the mature polypeptide and additional sequences, such as those encoding the about 28 amino acid residue leader or secretory sequence, such as a pre-, or pro- or prepro-protein sequence; the coding sequence of the mature polypeptide, with or without the aforementioned additional coding sequences.

Also encoded by nucleic acids of the invention are the above polypeptide sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and colleagues (Proc. Natl. Acad. Sci. USA 86:821-824 (1989)), for instance, hexa-histidine provides for convenient purification of the fusion protein. The “HA” tag is another peptide useful for purification which corresponds to an epitope derived from the influenza hemagglutinin protein, which has been described by Wilson and coworkers (Cell 37:767 (1984)). As discussed below, other such fusion proteins include the Dendriac or Brainiac-3 polypeptides fused to Fc at the N- or C-terminus.

Variant and Mutant Polynucleotides

The present invention further relates to variants of the nucleic acid molecules of the present invention, which encode portions, analogs or derivatives of the Dendriac and Brainiac-3 polypeptides. Variants may occur naturally, such as a natural allelic variant. By an “allelic variant” is intended one of several alternate forms of a gene occupying a given locus on a chromosome of an organism (Genes II, Lewin, B., ed., John Wiley & Sons, New York (1985)). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the Dendriac and Brainiac-3 polypeptides or portions thereof. Also especially preferred in this regard are conservative substitutions.

Most highly preferred are nucleic acid molecules encoding the mature polypeptide having the amino acid sequence shown in SEQ ID NO:2 and SEQ ID NO:10 or the mature Dendriac amino acid sequence encoded by the deposited cDNA clones.

Also most highly preferred are nucleic acid molecules encoding the mature polypeptide having the amino acid sequence shown in SEQ ID NO:4 and SEQ ID NO:12 or the mature Brainiac-3 amino acid sequence encoded by the deposited cDNA clones.

Thus, one embodiment of the invention provides an isolated nucleic acid molecule comprising a polynucleotide having a nucleotide sequence selected from the group consisting of: (a) a nucleotide sequence encoding the Dendriac polypeptide having the complete amino acid sequence in SEQ ID NO:2 and SEQ ID NO:10 (i.e., positions −25 to 294 of SEQ ID NO:2 and SEQ ID NO:10); (b) a nucleotide sequence encoding the Dendriac polypeptide having the complete amino acid sequence in SEQ ID NO:2 and SEQ ID NO:10 excepting the N-terminal methionine (i.e., positions −24 to 294 of SEQ ID NO:2 and SEQ ID NO:10); (c) a nucleotide sequence encoding the predicted mature Dendriac polypeptide having the amino acid sequence at positions 1-294 in SEQ ID NO:2 and SEQ ID NO:10; (d) a nucleotide sequence encoding the Dendriac polypeptide having the complete amino acid sequence encoded by the cDNA clone contained in ATCC Deposit Nos. 203056 and 209627; (e) a nucleotide sequence encoding the Dendriac polypeptide having the complete amino acid sequence excepting the N-terminal methionine encoded by the cDNA clone contained in ATCC Deposit Nos. 203056 and 209627; (f) a nucleotide sequence encoding the mature Dendriac polypeptide having the amino acid sequence encoded by the cDNA clone contained in ATCC Deposit Nos. 203056 and 209627; and (g) a nucleotide sequence complementary to any of the nucleotide sequences in (a), (b), (c), (d), (e) or (f), above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e), (f) or (g), above, or a polynucleotide which hybridizes under stringent hybridization conditions to a polynucleotide in (a), (b), (c), (d), (e), (f) or (g), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a Dendriac polypeptide having an amino acid sequence in (a), (b), (c), (d), (e) or (f), above. A further nucleic acid embodiment of the invention relates to an isolated nucleic acid molecule comprising a polynucleotide which encodes the amino acid sequence of a Dendriac polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a polynucleotide which encodes the amino acid sequence of a Dendriac polypeptide to have an amino acid sequence which contains not more than 10, 9, 8, 7, 6, 5, 4, 3, 2 or 1 conservative amino acid substitutions.

The present invention also relates to recombinant vectors, which include the isolated nucleic acid molecules of the present invention, and to host cells containing the recombinant vectors, as well as to methods of making such vectors and host cells and for using them for production of Dendriac polypeptides or peptides by recombinant techniques.

In addition, a further embodiment of the invention provides an isolated nucleic acid molecule comprising a polynucleotide having a nucleotide sequence selected from the group consisting of: (a) a nucleotide sequence encoding the Brainiac-3 polypeptide having the complete amino acid sequence in SEQ ID NO:4 and SEQ ID NO:12 (i.e., positions −28 to 324 of SEQ ID NO:4 and SEQ ID NO:12); (b) a nucleotide sequence encoding the Brainiac-3 polypeptide having the complete amino acid sequence in SEQ ID NO:4 and SEQ ID NO:12 excepting the N-terminal methionine (i.e., positions −27 to 324 of SEQ ID NO:4 and SEQ ID NO: 12); (c) a nucleotide sequence encoding the predicted mature Brainiac-3 polypeptide having the amino acid sequence at positions 1 to 324 in SEQ ID NO:4 and SEQ ID NO:12; (d) a nucleotide sequence encoding the Brainiac-3 polypeptide having the complete amino acid sequence encoded by the cDNA clone contained in ATCC Deposit Nos. 203451 and 209463; (e) a nucleotide sequence encoding the Brainiac-3 polypeptide having the complete amino acid sequence excepting the N-terminal methionine encoded by the cDNA clone contained in ATCC Deposit Nos. 203451 and 209463; (f) a nucleotide sequence encoding the mature Brainiac-3 polypeptide having the amino acid sequence encoded by the cDNA clone contained in ATCC Deposit Nos. 203451 and 209463; and (g) a nucleotide sequence complementary to any of the nucleotide sequences in (a), (b), (c), (d), (e) or (f), above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e), (f) or (g), above, or a polynucleotide which hybridizes under stringent hybridization conditions to a polynucleotide in (a), (b), (c), (d), (e), (f) or (g), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a Brainiac-3 polypeptide having an amino acid sequence in (a), (b), (c), (d), (e) or (f), above. A further nucleic acid embodiment of the invention relates to an isolated nucleic acid molecule comprising a polynucleotide which encodes the amino acid sequence of a Brainiac-3 polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a polynucleotide which encodes the amino acid sequence of a Brainiac-3 polypeptide to have an amino acid sequence which contains not more than 10, 9, 8, 7, 6, 5, 4, 3, 2 or 1 conservative amino acid substitutions.

The present invention also relates to recombinant vectors, which include the isolated nucleic acid molecules of the present invention, and to host cells containing the recombinant vectors, as well as to methods of making such vectors and host cells and for using them for production of Brainiac-3 polypeptides or peptides by recombinant techniques.

By a polynucleotide having a nucleotide sequence at least, for example, 95% identical to a reference nucleotide sequence encoding a Dendriac or Brainiac-3 polypeptide is intended that the nucleotide sequence of the polynucleotide is identical to the reference sequence except that the polynucleotide sequence may include up to five point mutations per each 100 nucleotides of the reference nucleotide sequences encoding the Dendriac or Brainiac-3 polypeptides. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequences shown in FIGS. 1A, 1B, and 1C or 2A and 2B or to the nucleotides sequence of the deposited cDNA clones can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman to find the best segment of homology between two sequences (Advances in Applied Mathematics 2:482-489 (1981)). When using Bestfit or any other sequence alignment program to determine whether a particular sequence is, for instance, 95% identical to a reference sequence according to the present invention, the parameters are set, of course, such that the percentage of identity is calculated over the fill length of the reference nucleotide sequence and that gaps in homology of up to 5% of the total number of nucleotides in the reference sequence are allowed. A preferred method for determining the best overall match between a query sequence (a sequence of the present invention) and a subject sequence, also referred to as a global sequence alignment, can be determined using the FASTDB computer program based on the algorithm of Brutlag and colleagues (Comp. App. Biosci. 6:237-245 (1990)). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=l, Joining Penalty=30, Randomization Group Length=0, Cutoff Score=l, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

The present application is directed to nucleic acid molecules at least 90%, 95%, 96%, 97%, 98% or 99% identical to the nucleic acid sequence shown in FIGS. 1A, 1B, and 1C (SEQ ID NO: 1) and SEQ ID NO:9 or to the nucleic acid sequence of the deposited cDNA, irrespective of whether they encode a polypeptide having Dendriac activity. This is because even where a particular nucleic acid molecule does not encode a polypeptide having Dendriac activity, one of skill in the art would still know how to use the nucleic acid molecule, for instance, as a hybridization probe or a polymerase chain reaction (PCR) primer. Uses of the nucleic acid molecules of the present invention that do not encode a polypeptide having Dendriac activity include, inter alia, (1) isolating the Dendriac gene or allelic variants thereof in a cDNA library; (2) in situ hybridization (e.g., “FISH”) to metaphase chromosomal spreads to provide precise chromosomal location of the Dendriac gene, as described by Verma and colleagues (Human Chromosomes: A Manual of Basic Techniques, Pergamon Press, New York (1988)); and Northern Blot analysis for detecting Dendriac MRNA expression in specific tissues.

The present application is also directed to nucleic acid molecules at least 90%, 95%, 96%, 97%, 98% or 99% identical to the nucleic acid sequence shown in FIGS. 2A and 2B (SEQ ID NO:3) and SEQ ID NO:11 or to the nucleic acid sequence of the deposited cDNA, irrespective of whether they encode a polypeptide having Brainiac-3 activity. This is because even where a particular nucleic acid molecule does not encode a polypeptide having Brainiac-3 activity, one of skill in the art would still know how to use the nucleic acid molecule, for instance, as a hybridization probe or a polymerase chain reaction (PCR) primer. Uses of the nucleic acid molecules of the present invention that do not encode a polypeptide having Brainiac-3 activity include, inter alia, (1) isolating the Brainiac-3 gene or allelic variants thereof in a cDNA library; (2) in situ hybridization (e.g., “FISH”) to metaphase chromosomal spreads to provide precise chromosomal location of the Brainiac-3 gene, as described by Verma and colleagues (Human Chromosomes: A Manual of Basic Techniques, Pergamon Press, New York (1988)); and Northern Blot analysis for detecting Brainiac-3 MRNA expression in specific tissues.

Preferred, however, are nucleic acid molecules having sequences at least 90%, 95%, 96%, 97%, 98% or 99% identical to the nucleic acid sequence shown in FIGS. 1A, 1B, and 1C (SEQ ID NO:1) and SEQ ID NO:9 or to the nucleic acid sequence of the deposited cDNAs which do, in fact, encode a polypeptide having Dendriac polypeptide activity. By “a polypeptide having Dendriac polypeptide activity” is intended polypeptides exhibiting activity similar, but not necessarily identical, to an activity of the mature Dendriac polypeptide of the invention, as measured in a particular biological assay.

Also preferred are nucleic acid molecules having sequences at least 90%, 95%, 96%, 97%, 98% or 99% identical to the nucleic acid sequence shown in FIGS. 2A and 2B (SEQ ID NO:3) and SEQ ID NO: 11 or to the nucleic acid sequence of the deposited cDNAs which do, in fact, encode a polypeptide having Brainiac-3 polypeptide activity. By “a polypeptide having Brainiac-3 polypeptide activity” is intended polypeptides exhibiting activity similar, but not necessarily identical, to an activity of the mature Brainiac-3 polypeptide of the invention, as measured in a particular biological assay.

For example, the Dendriac and Brainiac-3 polypeptides of the present invention modulate cellular growth and differentiation. Thus, biological activity of Dendriac and/or Brainiac-3 polypeptides can be examined in organ culture assays or in colony assay systems in agarose culture. Stimulation or inhibition of cellular proliferation may be measured by a variety of assays. For observing cell growth inhibition, one can use a solid or liquid medium. In a solid medium, cells undergoing growth inhibition can easily be selected from the subject cell group by comparing the sizes of colonies formed. In a liquid medium, growth inhibition can be screened by measuring culture broth turbity or incorporation of labeled thymidine in DNA. Typically, the incorporation of a nucleoside analog into newly synthesized DNA is employed to measure proliferation (i.e., active cell growth) in a population of cells. For example, bromodeoxyuridine (BrdU) can be employed as a DNA labeling reagent and anti-BrdU mouse monoclonal antibody (clone BMC 9318 IgG₁) can be employed as a detection reagent. This antibody binds only to cells containing DNA which has incorporated bromodeoxyuridine. A number of detection methods may be used in conjunction with this assay including immunofluorescence, imnmunohistochemical, ELISA, and calorimetric methods. Kits that include bromodeoxyuridine (BrdU) and anti-BrdU mouse monoclonal antibody are commercially available from Boehringer Mannheim (Indianapolis, Ind.).

The effect upon cellular differentiation can be measured by contacting embryonic cells with various amounts of a Dendriac and/or Brainiac-3 polypeptide and observing the effect upon differentiation of the embryonic cells. Tissue-specific antibodies and microscopy may be used to identify the resulting cells.

Dendriac polypeptides modulate immune and/or nervous system cell proliferation and differentiation in a dose-dependent manner in the above-described assays. Thus, “a polypeptide having Dendriac polypeptide activity” includes polypeptides that also exhibit any of the same growth and differentiation regulating activities in the above-described assays in a dose-dependent manner. Although the degree of dose-dependent activity need not be identical to that of the Dendriac polypeptide, preferably, a polypeptide having Dendriac polypeptide activity will exhibit substantially similar dose-dependence in a given activity as compared to the Dendriac polypeptide (i.e., the candidate polypeptide will exhibit greater activity or not more than about 25-fold less and, preferably, not more than about tenfold less activity relative to the reference Dendriac polypeptide).

Brainiac-3 polypeptides modulates immune and/or nervous system cell proliferation and differentiation in a dose-dependent manner in the above-described assay. Thus, “a polypeptide having Brainiac-3 activity” includes polypeptides that also exhibit any of the same growth and differentiation regulating activities in the above-described assays in a dose-dependent manner. Although the degree of dose-dependent activity need not be identical to that of the Brainiac-3 polypeptide, preferably, “a polypeptide having Brainiac-3 polypeptide activity will” exhibit substantially similar dose-dependence in a given activity as compared to the Brainiac-3 polypeptide (i.e., the candidate polypeptide will exhibit greater activity or not more than about 25-fold less and, preferably, not more than about tenfold less activity relative to the reference Brainiac-3 polypeptide).

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNAs or the nucleic acid sequence shown in FIGS. 1A, 1B, and 1C (SEQ ID NO:1) and SEQ ID NO:9 will encode a polypeptide having Dendriac polypeptide activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having Dendriac polypeptide activity. One of ordinary skill in the art will also immediately recognize that a large number of the nucleic acid molecules having a sequence at least 90%, 95%, 96%, 97%, 98%, or 99% identical to the nucleic acid sequence of the deposited cDNAs or the nucleic acid sequence shown in FIGS. 2A and 2B (SEQ ID NO:3) and SEQ ID NO: 11 will encode a polypeptide “having Brainiac-3 polypeptide activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having either Dendriac or Brainiac-3 polypeptide activity. This is because the skilled artisan is fully aware of amino acid substitutions that are either less likely or not likely to significantly effect polypeptide function (e.g., replacing one aliphatic amino acid with a second aliphatic amino acid), as further described below.

Vectors and Host Cells

The present invention also relates to vectors which include the isolated DNA molecules of the present invention, host cells which are genetically engineered with the recombinant vectors, and the production of Dendriac or Brainiac-3 polypeptides or fragments thereof by recombinant techniques. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp, phoa and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Vectors preferred for use in bacteria include pHE4-5 (ATCC Accession No. 209311; and variations thereof), pQE70, pQE60 and pQE-9 (QIAGEN, Inc., supra); pBS vectors, Phagescript vectors, Bluescript vectors, pNH8A, pNH16a, pNH18A, pNH46A (Stratagene); and ptrc99a, pKK223-3, pKK233-3, pDR540, pRIT5 (Pharmacia). Among preferred eukaryotic vectors are pWLNEO, pSV2CAT, pOG44, pXT1, and pSG (Stratagene); and pSVK3, pBPV, pMSG and pSVL (Pharmacia). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals (for example, Davis, et al, Basic Methods In Molecular Biology (1986)).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify polypeptides. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5 (Bennett, D., et al., J. Molecular Recognition 8:52-58 (1995); Johanson, K., et al., J. Biol. Chem. 270:9459-9471 (1995)).

The Dendriac and Brainiac-3 polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

### Polypeptides and Fragments

The invention further provides an isolated Dendriac polypeptide having the amino acid sequence encoded by the deposited cDNAs, or the amino acid sequence in SEQ ID NO:2 and SEQ ID NO:10, or a peptide or polypeptide comprising a portion of the above polypeptides. The invention also provides an isolated Brainiac-3 polypeptide having the amino acid sequence encoded by the deposited cDNAs, or the amino acid sequence in SEQ ID NO:4 and SEQ ID NO:12, or a peptide or polypeptide comprising a portion of the above polypeptides.

Variant and Mutant Polypeptides

To improve or alter the characteristics of Dendriac and Brainiac-3 polypeptides, protein engineering may be employed. Recombinant DNA technology known to those skilled in the art can be used to create novel mutant polypeptides or muteins including single or multiple amino acid substitutions, deletions, additions or fusion polypeptides. Such modified polypeptides can show, e.g., enhanced activity or increased stability. In addition, they may be purified in higher yields and show better solubility than the corresponding natural polypeptide, at least under certain purification and storage conditions.

N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron and colleagues (J. Biol. Chem., 268:2984-2988 (1993)) reported modified KGF proteins that had heparin binding activity even if 3, 8, or 27 N-terminal amino acid residues were missing.

In the present case, since the Dendriac polypeptide of the invention is a member of the Brainiac polypeptide family, deletions of N-terminal amino acids up to the arginine at position 57 of SEQ ID NO:2 and SEQ ID NO:10 may retain some biological activity such as the ability to modulate cell growth and differentiation. Polypeptides having further N-terminal deletions including the arginine-57 residue in SEQ ID NO:2 and SEQ ID NO:10 would not be expected to retain such biological activities because it is known that this residue in a Brainiac-related polypeptide is in the beginning of the conserved domain believed to be required for biological activities.

However, even if deletion of one or more amino acids from the N-terminus of a protein results in modification or loss of one or more biological functions of the protein, other biological activities may still be retained. Thus, the ability of the shortened polypeptide to induce and/or bind to antibodies which recognize the complete or mature form of the polypeptide generally will be retained when less than the majority of the residues of the complete or mature form of the polypeptide are removed from the N-terminus. Whether a particular polypeptide lacking N-terminal residues of a complete polypeptide retains such immunologic activities can readily be determined by routine methods described herein and otherwise known in the art.

Accordingly, the present invention further provides polypeptides having one or more residues deleted from the amino terminus of the amino acid sequence of the Dendriac polypeptide shown in SEQ ID NO:2 and in SEQ ID NO:10, up to the arginine residue at position number 57, and polynucleotides encoding such polypeptides. In particular, the present invention provides polypeptides comprising the amino acid sequence of residues n¹-294 of SEQ ID NO:2 and SEQ ID NO:10, where n¹ is an integer in the range of −25 to 56, and 57 is the position of the first residue from the N-terminus of the complete Dendriac polypeptide (shown in SEQ ID NO:2 and in SEQ ID NO:10) believed to be required for modulation of cell growth and differentiation activity of the Dendriac polypeptide.

More in particular, the invention provides polynucleotides encoding polypeptides having the amino acid sequence of residues of −25-294, −24-294, −23-294, −22-294, −21-294, −20-294, −19-294, −18-294, −17-294, −16-294, −15-294, −14-294, −13-294, −12-294, −11-294, −10-294, −9-294, −8-294, −7-294, −6-294, −5-294, −4-294, −3-294, −2-294, −1-294, 1-294, 2-294, 3-294, 4-294, 5-294, 6-294, 7-294, 8-294, 9-294, 10-294, 11-294, 12-294, 13-294, 14-294, 15-294, 16-294, 17-294, 18-294, 19-294, 20-294, 21-294, 22-294, 23-294, 24-294, 25-294, 26-294, 27-294, 28-294, 29-294, 30-294, 31-294, 32-294, ;33-294, 34-294, 35-294, 36-294, 37-294, 38-294, 39-294, 40-294, 41-294, 42-294, 43-294, 44-294, 45-294, 46-294, 47-294, 48-294, 49-294, 50-294, 51-294, 52-294, 53-294, 54-294, 55-294, and 56-294 of SEQ ID NO:2 and of SEQ ID NO: 10. Polynucleotides encoding these polypeptides also are provided.

Similarly, many examples of biologically functional C-terminal deletion muteins are known. For instance, Interferon gamma shows up to ten times higher activities by deleting 8-10 amino acid residues from the carboxy terminus of the protein (Dobeli, et al, J. Biotechnology 7:199-216 (1988)).

In the present case, since the Dendriac polypeptide of the invention is a member of the Brainiac polypeptide family, deletions of C-terminal amino acids up to the cysteine at position 292 of SEQ ID NO:2 and SEQ ID NO:10 may retain some biological activity such as the ability to modulate cell growth and differentiation. Polypeptides having further C-terminal deletions including the cysteine residue at position 292 of SEQ ID NO:2 and SEQ ID NO: 10 would not be expected to retain such biological activities because this residue is in the beginning of the conserved domain required for biological activities.

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the polypeptide generally will be retained when less than the majority of the residues of the complete or mature form of the polypeptide are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues from the carboxy terminus of the amino acid sequence of the Dendriac polypeptide shown in SEQ ID NO:2 and in SEQ ID NO:10, up to the cysteine residue at position 292 of SEQ ID NO:2 and of SEQ ID NO: 10, and polynucleotides encoding such polypeptides. In particular, the present invention provides polypeptides having the amino acid sequence of residues −25-m¹ of the amino acid sequence in SEQ ID NO:2 and in SEQ ID NO:10, where m¹ is any integer in the range of 292 to 294, and residue 292 is the position of the first residue from the C-terminus of the complete Dendriac polypeptide (shown in SEQ ID NO:2 and in SEQ ID NO:10) believed to be required for the cell growth and differentiation modulatory activities of the Dendriac polypeptide.

More in particular, the invention provides polynucleotides encoding polypeptides having the amino acid sequence of residues −25-292, −25-293, and −25-294 of SEQ ID NO:2 and of SEQ ID NO:10. Polynucleotides encoding these polypeptides also are provided.

The invention also provides polypeptides having one or more amino acids deleted from both the amino and the carboxyl termini, which may be described generally as having residues n¹ -m¹ of SEQ ID NO:2 and SEQ ID NO: 10, where n¹ and m¹ are integers as described above.

Also included are a nucleotide sequence encoding a polypeptide consisting of a portion of the complete Dendriac amino acid sequence encoded by the cDNA clone contained in ATCC Deposit Nos. 203056 and 209627, where this portion excludes from 1 to about 81 amino acids from the amino terminus of the complete amino acid sequence encoded by the cDNA clone contained in ATCC Deposit Nos. 203056 and 209627, or from 1 to about 3 amino acids from the carboxy terminus, or any combination of the above amino terminal and carboxy terminal deletions, of the complete amino acid sequence encoded by the cDNA clones contained in ATCC Deposit Nos. 203056 and 209627.

Also in the present case, since the Brainiac-3 polypeptide of the invention is a member of the Brainiac polypeptide family, deletions of N-terminal amino acids up to the arginine at position 81 of SEQ ID NO:4 and SEQ ID NO: 12 may retain some biological activity such as the ability to modulate cell growth and differentiation. Polypeptides having further N-terminal deletions including the arginine-81 residue in SEQ ID NO:4 and SEQ ID NO:12 would not be expected to retain such biological activities because it is known that this residue in a Brainiac-related polypeptide is in the beginning of the conserved domain believed to be required for biological activities.

Accordingly, the present invention further provides polypeptides having one or more residues deleted from the amino terminus of the amino acid sequence of the Brainiac-3 polypeptide shown in SEQ ID NO:4 and in SEQ ID NO:12, up to the arginine residue at position number 81, and polynucleotides encoding such polypeptides. In particular, the present invention provides polypeptides comprising the amino acid sequence of residues n²-324 of SEQ ID NO:4 and SEQ ID NO:12, where n² is an integer in the range of −28 to 80, and 81 is the position of the first residue from the N-terminus of the complete Brainiac-3 polypeptide (shown in SEQ ID NO:4 and in SEQ ID NO: 12) believed to be required for modulation of cell growth and differentiation activity of the Brainiac-3 polypeptide.

More in particular, the invention provides polynucleotides encoding polypeptides having the amino acid sequence of residues of −28-324, −27-324, −26-324, −25-324, −24-324, −23-324, −22-324, −21-324, −20-324, −19-324, −18-324, −17-324, −16-324, −15-324, −14-324, −13-324, −12-324, −11-324, −10-324, −9-324, −8-324, −7-324, −6-324, −5-324, −4-324, −3-324, −2-324, −1-324, 1-324, 2-324, 3-324, 4-324, 5-324, 6-324, 7-324, 8-324, 9-324, 10-324, 11-324, 12-324, 13-324, 14-324, 15-324, 16-324, 17-324, 18-324, 19-324, 20-324, 21-324, 22-324, 23-324, 24-324, 25-324, 26-324, 27-324, 28-324, 29-324, 30-324, 31-324, 32-324, 33-324, 34-324, 35-324, 36-324, 37-324, 38-324, 39-324, 40-324, 41-324, 42-324, 43-324, 44-324, 45-324, 46-324, 47-324, 48-324, 49-324, 50-324, 51-324, 52-324, 53-324, 54-324, 55-324, 56-324, 57-324, 58-324, 59-324, 60-324, 61-324, 62-324, 63-324, 64-324, 65-324, 66-324, 67-324, 68-324, 69-324, 70-324, 71-324, 72-324, 73-324, 74-324, 75-324, 76-324, 77-324, 78-324, 79-324, and 80-324 of SEQ ID NO:4 and of SEQ ID NO:12. Polynucleotides encoding these polypeptides also are provided.

Also, in the present case, since the Brainiac-3 polypeptide of the invention is a member of the Brainiac polypeptide family, deletions of C-terminal amino acids up to the cysteine at position 316 of SEQ ID NO:4 and SEQ ID NO: 12 may retain some biological activity such as the ability to modulate cell growth and differentiation. Polypeptides having further C-terminal deletions including the cysteine residue at position 316 of SEQ ID NO:4 and SEQ ID NO:12 would not be expected to retain such biological activities because this residue is in the beginning of the conserved domain required for biological activities.

Accordingly, the present invention further provides polypeptides having one or more residues from the carboxy terminus of the amino acid sequence of the Brainiac-3 polypeptide shown in SEQ ID NO:4 and in SEQ ID NO:12, up to the cysteine residue at position 316 of SEQ ID NO:4 and of SEQ ID NO:12, and polynucleotides encoding such polypeptides. In particular, the present invention provides polypeptides having the amino acid sequence of residues −28-m² of the amino acid sequence in SEQ ID NO:4 and SEQ ID NO:12, where m² is any integer in the range of316 to 324, and residue 316 is the position of the first residue from the C-terminus of the complete Brainiac-3 polypeptide (shown in SEQ ID NO:4 and in SEQ ID NO:12) believed to be required for the cell growth and differentiation modulatory activities of the Brainiac-3 polypeptide.

More in particular, the invention provides polynucleotides encoding polypeptides having the amino acid sequence of residues −28-324, −28-323, −28-322, −28-321, −28-320, −28-319, −28-318, and -28-317 of SEQ ID NO:4 and of SEQ ID NO:12. Polynucleotides encoding these polypeptides also are provided.

The invention also provides polypeptides having one or more amino acids deleted from both the amino and the carboxyl termini, which may be described generally as having residues n²-m² of SEQ ID NO:4 and SEQ ID NO: 12, where n² and m² are integers as described above.

Also included are a nucleotide sequence encoding a polypeptide consisting of a portion of the complete Brainiac-3 amino acid sequence encoded by the cDNA clone contained in ATCC Deposit Nos. 203451 and 209463, where this portion excludes from 1 to about 108 amino acids from the amino terminus of the complete amino acid sequence encoded by the cDNA clone contained in ATCC Deposit Nos. 203451 and 209463, or from 1 to about 8 amino acids from the carboxy terminus, or any combination of the above amino terminal and carboxy terminal deletions, of the complete amino acid sequence encoded by the cDNA clones contained in ATCC Deposit Nos. 203451 and 209463. Polynucleotides encoding all of the above deletion mutant polypeptide forms also are provided.

As mentioned above, even if deletion of one or more amino acids from the N-terminus of a protein results in modification of loss of one or more biological functions of the protein, other biological activities may still be retained. Thus, the ability of shortened Dendriac and Brainiac-3 muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a Dendriac or Brainiac-3 mutein with a large number of deleted N-terminal amino acid residues may retain some biological or immunogenic activities. In fact, peptides composed of as few as six Dendriac or Brainiac-3 amino acid residues may often evoke an immune response.

Accordingly, the present invention further provides polypeptides having one or more residues deleted from the amino terminus of the Dendriac amino acid sequence shown in FIGS. 1A, 1B, and 1C (i.e., SEQ ID NO:2 and in SEQ ID NO:10), up to the asparagine residue at position number 314 and polynucleotides encoding such polypeptides. In particular, the present invention provides polypeptides comprising the amino acid sequence of residues n³-314 of FIGS. 1A, 1B, and 1C (SEQ ID NO:2 and SEQ ID NO:10), where n³ is an integer in the range of 2 to 314, and 315 is the position of the first residue from the N-terminus of the complete Dendriac polypeptide believed to be required for at least immunogenic activity of the Dendriac polypeptide.

More in particular, the invention provides polynucleotides encoding polypeptides comprising, or alternatively consisting of, the amino acid sequence of residues of S-2 to Y-319; L-3 to Y-319; R-4 to Y-319; S-5 to Y-319; L-6 to Y-319; K-7 to Y-319; W-8 to Y-319; S-9 to Y-319; L-10 to Y-319; L-11 to Y-319; L-12 to Y-319; L-13 to Y-319; S-14 to Y-319; L-15 to Y-319; L-16 to Y-319; S-17 to Y-319; F-18 to Y-319; F-19 to Y-319; V-20 to Y-319; M-21 to Y-319; W-22 to Y-319; Y-23 to Y-319; L-24 to Y-319; S-25 to Y-319; L-26 to Y-319; P-27 to Y-319; H-28 to Y-319; Y-29 to Y-319; N-30 to Y-319; V-31 to Y-319; I-32 to Y-319; E-33 to Y-319; R-34 to Y-319; V-35 to Y-319; N-36 to Y-319; W-37 to Y-319; M-38 to Y-319; Y-39 to Y-319; F-40 to Y-319; Y-41 to Y-319; E-42 to Y-319; Y-43 to Y-319; E-44 to Y-319; P-45 to Y-319; I-46 to Y-319; Y-47 to Y-319; R-48 to Y-319; Q-49 to Y-319; D-50 to Y-319; F-51 to Y-319; H-52 to Y-319; F-53 to Y-319; T-54 to Y-319; L-55 to Y-319; R-56 to Y-319; E-57 to Y-319; H-58 to Y-319; S-59 to Y-319; N-60 to Y-319; C-61 to Y-319; S-62 to Y-319; H-63 to Y-319; Q-64 to Y-319; N-65 to Y-319; P-66 to Y-319; F-67 to Y-319; L-68 to Y-319; V-69 to Y-319; I-70 to Y-319; L-71 to Y-319; V-72 to Y-319; T-73 to Y-319; S-74 to Y-319; H-75 to Y-319; P-76 to Y-319; S-77 to Y-319; D-78 to Y-319; V-79 to Y-319; K-80 to Y-319; A-81 to Y-319; R-82 to Y-319; Q-83 to Y-319; A-84 to Y-319; I-85 to Y-319; R-86 to Y-319; V-87 to Y-319; T-88 to Y-319; W-89 to Y-319; G-90 to Y-319; E-91 to Y-319; K-92 to Y-319; K-93 to Y-319; S-94 to Y-319; W-95 to Y-319; W-96 to Y-319; G-97 to Y-319; Y-98 to Y-319; E-99 to Y-319; V-100 to Y-319; L-101 to Y-319; T-102 to Y-319; F-103 to Y-319; F-104 to Y-319; L-1O5 to Y-319; L-106 to Y-319; G-107 to Y-319; Q-108 to Y-319; E-109 to Y-319; A-110 to Y-319; E-111 to Y-319; K-112 to Y-319; E-113 to Y-319; D-114 to Y-319; K-115 to Y-319; M-116 to Y-319; L-117 to Y-319; A-118 to Y-319; L-119 to Y-319; S-120 to Y-319; L-121 to Y-319; E-122 to Y-319; D-123 to Y-319; E-124 to Y-319; H-125 to Y-319; L-126 to Y-319; L-127 to Y-319; Y-128 to Y-319; G-129 to Y-319; D-130 to Y-319; I-131 to Y-319; 1-132 to Y-319; R-133 to Y-319; Q-134 to Y-319; D-135 to Y-319; F-136 to Y-319; L-137 to Y-319; D-138 to Y-319; T-139 to Y-319; Y-140 to Y-319; N-141 to Y-319; N-142 to Y-319; L-143 to Y-319; T-144 to Y-319; L-145 to Y-319; K-146 to Y-319; T-147 to Y-319; I-148 to Y-319; M-149 to Y-319; A-150 to Y-319; F-151 to Y-319; R-152 to Y-319; W-153 to Y-319; V-154 to Y-319; T-155 to Y-319; E-156 to Y-319; F-157 to Y-319; C-158 to Y-319; P-159 to Y-319; N-160 to Y-319; A-161 to Y-319; K-162 to Y-319; Y-163 to Y-319; V-164 to Y-319; M-165 to Y-319; K-166 to Y-319; T-167 to Y-319; D-168 to Y-319; T-169 to Y-319; D-170 to Y-319; V-171 to Y-319; F-172 to Y-319; I-173 to Y-319; N-174 to Y-319; T-175 to Y-319; G-176 to Y-319; N-177 to Y-319; L-178 to Y-319; V-179 to Y-319; K-190 to Y-319; Y-181 to Y-319; L-182 to Y-319; L-183 to Y-319; N-184 to Y-319; L-185 to Y-319; N-186 to Y-319; H-187 to Y-319; S-188 to Y-319; E-189 to Y-319; K-190 to Y-319; F-191 to Y-319; F-192 to Y-319; T-193 to Y-319; G-194 to Y-319; Y-195 to Y-319; P-196 to Y-319; L-197 to Y-319; I-198 to Y-319; D-199 to Y-319; N-200 to Y-319; Y-201 to Y-319; S-202 to Y-319; Y-203 to Y-319; R-204 to Y-319; G-205 to Y-319; F-206 to Y-319; Y-207 to Y-319; Q-208 to Y-319; K-209 to Y-319; T-210 to Y-319; H-211 to Y-319; I-212 to Y-319; S-213 to Y-319; Y-214 to Y-319; Q-215 to Y-319; E-216 to Y-319; Y-217 to Y-319; P-218 to Y-319; F-219 to Y-319; K-220 to Y-319; V-221 to Y-319; F-222 to Y-319; P-223 to Y-319; P -224 to Y-319; Y-225 to Y-319; C-226 to Y-319; S-227 to Y-319; G-228 to Y-319; L-229 to Y-319; G-230 to Y-319; Y-231 to Y-319; I-232 to Y-319; M-233 to Y-319; S-234 to Y-319; R-235 to Y-319; D-236 to Y-319; L-237 to Y-319; V-238 to Y-319; P-239 to Y-319; R-240 to Y-319; 1-241 to Y-319; Y-242 to Y-319; E-243 to Y-319; M-244 to Y-319; M-245 to Y-319; G-246 to Y-319; H-247 to Y-319; V-248 to Y-319; K-249 to Y-319; P-250 to Y-319; I-251 to Y-319; K-252 to Y-319; F-253 to Y-319; E-254 to Y-319; D-255 to Y-319; V-256 to Y-319; Y-257 to Y-319; V-258 to Y-319; G-259 to Y-319; I-260 to Y-319; C-261 to Y-319; L-262 to Y-319; N-263 to Y-319; L-264 to Y-319; L-265 to Y-319; K-266 to Y-319; V-267 to Y-319; N-268 to Y-319; I-269 to Y-319; H-270 to Y-319; I-271 to Y-319; P-272 to Y-319; E-273 to Y-319; D-274 to Y-319; T-275 to Y-319; N-276 to Y-319; L-277 to Y-319; F-278 to Y-319; F-279 to Y-319; L-280 to Y-319; Y-281 to Y-319; R-282 to Y-319; I-283 to Y-319; H-284 to Y-319; L-285 to Y-319; D-286 to Y-319; V-287 to Y-319; C-288 to Y-319; Q-289 to Y-319; L-290 to Y-319; R-291 to Y-319; R-292 to Y-319; V-293 to Y-319; 1-294 to Y-319; A-295 to Y-319; A-296 to Y-319; H-297 to Y-319; G-298 to Y-319; F-299 to Y-319; S-300 to Y-319; S-301 to Y-319; K-302 to Y-319; E-303 to Y-319; I-304 to Y-319; 1-305 to Y-319; T-306 to Y-319; F-307 to Y-319; W-308 to Y-319; Q-309 to Y-319; V-310 to Y-319; M-311 to Y-319; L-312 to Y-319; R-313 to Y-319; and N-314 to Y-319 of the Dendriac sequence shown in FIGS. 1A, 1B, and 1C (which is identical to the sequence shown as SEQ ID NO:2 and in SEQ ID NO: 10, with the exception that the amino acid residues in FIGS. 1A, 1B, and 1C are numbered consecutively from 1 through 319 from the N-terminus to the C-terminus, while the amino acid residues in SEQ ID NO:2 and in SEQ ID NO:10 are numbered consecutively from −25 through 294 to reflect the position of the predicted signal peptide). Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened Dendriac mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a Dendriac mutein with a large number of deleted C-terminal amino acid residues may retain some biological or immunogenic activities. In fact, peptides composed of as few as six Dendriac amino acid residues may often evoke an immune response.

Accordingly, the present invention further provides polypeptides having one or more residues deleted from the carboxy terminus of the amino acid sequence of the Dendriac polypeptide shown in FIGS. 1A, 1B, and 1C (SEQ ID NO:2 and SEQ ID NO: 10), up to the leucine residue at position number 6, and polynucleotides encoding such polypeptides. In particular, the present invention provides polypeptides comprising the amino acid sequence of residues 1-m³ of FIGS. 1A, 1B, and 1C (i.e., SEQ ID NO:2 and SEQ ID NO: 10), where m³ is an integer in the range of 6 to 319, and 6 is the position of the first residue from the C-terminus of the complete Dendriac polypeptide believed to be required for at least immunogenic activity of the Dendriac polypeptide.

More in particular, the invention provides polynucleotides encoding polypeptides comprising, or alternatively consisting of, the amino acid sequence of residues M-1 to H-318; M-1 to C-317; M-1 to T-316; M-1 to T-315; M-1 to N-314; M-1 to R-313; M-1 to L-312; M-1 to M-311; M-1 to V-310; M-1 to Q-309; M-1 to W-308; M-1 to F-307; M-1 to T-306; M-1 to I-305; M-1 to I-304; M-1 to E-303; M-1 to K-302; M-1 to S-301; M-1 to S-300; M-1 to F-299; M-1 to G-298; M-1 to H-297; M-1 to A-296; M-1 to A-295; M-1 to I-294; M-1 to V-293; M-1 to R-292; M-1 to R-291; M-1 to L-290; M-1 to Q-289; M-1 to C-288; M-1 to V-287; M-1 to D-286; M-1 to L-285; M-1 to H-284; M-1 to I-283; M-1 to R-282; M-1 to Y-281; M-1 to L-280; M-1 to F-279; M-1 to F-278; M-1 to L-277; M-1 to N-276; M-1 to T-275; M-1 to D-274; M-1 to E-273; M-1 to P-272; M-1 to I-271; M-1 to H-270; M-1 to I-269; M-1 to N-268; M-1 to V-267; M-1 to K-266; M-1 to L-265; M-1 to L-264; M-1 to N-263; M-1 to L-262; M-1 to C-261; M-1 to I-260; M-1 to G-259; M-1 to V-258; M-1 to Y-257; M-1 to V-256; M-1 to D-255; M-1 to E-254; M-1 to F-253; M-1 to K-252; M-1 to I-251; M-1 to P-250; M-1 to K-249; M-1 to V-248; M-1 to H-247; M-1 to G-246; M-1 to M-245; M-1 to M-244; M-1 to E-243; M-1 to Y-242; M-1 to I-241; M-1 to R-240; M-1 to P-239; M-1 to V-238; M-1 to L-237; M-1 to D-236; M-1 to R-235; M-1 to S-234; M-1 to M-233; M-1 to I-232; M-1 to Y-231; M-1 to G-230; M-1 to L-229; M-1 to G-228; M-1 to S-227; M-1 to C-226; M-1 to Y-225; M-1 to P-224; M-1 to P-223; M-1 to F-222; M-1 to V-221; M-1 to K-220; M-1 to F-219; M-1 to P-218; M-1 to Y-217; M-1 to E-216; M-1 to Q-215; M-1 to Y-214; M-1 to S-213; M-1 to I-212; M-1 to H-211; M-1 to T-210; M-1 to K-209; M-1 to Q-208; M-1 to Y-207; M-1 to F-206; M-1 to G-205; M-1 to R-204; M-1 to Y-203; M-1 to S-202; M-1 to Y-201; M-1 to N-200; M-1 to D-199; M-1 to I-198; M-1 to L-197; M-1 to P-196; M-1 to Y-195; M-1 to G-194; M-1 to T-193; M-1 to F-192; M-1 to F-191; M-1 to K-190; M-1 to E-189; M-1 to S-188; M-1 to H-187; M-1 to N-186; M-1 to L-185; M-1 to N-184; M-1 to L-183; M-1 to L-182; M-1 to Y-181; M-1 to K-180; M-1 to V-179; M-1 to L-178; M-1 to N-177; M-1 to G-176; M-1 to T-175; M-1 to N-174; M-1 to I-173; M-1 to F-172; M-1 to V-171; M-1 to D-170; M-1 to T-169; M-1 to D-168; M-1 to T-167; M-1 to K-166; M-1 to M-165; M-1 to V-164; M-1 to Y-163; M-1 to K-162; M-1 to A-161; M-1 to N-160; M-1 to P-159; M-1 to C-158; M-1 to F-157; M-1 to E-156; M-1 to T-155; M-1 to V-154; M-1 to W-153; M-1 to R-152; M-1 to F-151; M-1 to A-150; M-1 to M-149; M-1 to I-148; M-1 to T-147; M-1 to K-146; M-1 to L-145; M-1 to T-144; M-1 to L-143; M-1 to N-142; M-1 to N-141; M-1 to Y-140; M-1 to T-139; M-1 to D-138; M-1 to L-137; M-1 to F-136; M-1 to D-135; M-1 to Q-134; M-1 to R-133; M-1 to I-132; M-1 to I-131; M-1 to D-130; M-1 to G-129; M-1 to Y-128; M-1 to L-127; M-1 to L-126; M-1 to H-125; M-1 to E-124; M-1 to D-123; M-1 to E-122; M-1 to L-121; M-1 to S-120; M-1 to L-119; M-1 to A-118; M-1 to L-117; M-1 to M-116; M-1 to K-115; M-1 to D-114; M-1 to E-113; M-1 to K-112; M-1 to E-111; M-1 to A-110; M-1 to E-109; M-1 to Q-108; M-1 to G-107; M-1 to L-106; M-1 to L-105; M-1 to F-104; M-1 to F-103; M-1 to T-102; M-1 to L-101; M-1 to V-100; M-1 to E-99; M-1 to Y-98; M-1 to G-97; M-1 to W-96; M-1 to W-95; M-1 to S-94; M-1 to K-93; M-1 to K-92; M-1 to E-91; M-1 to G-90; M-1 to W-89; M-1 to T-88; M-1 to V-87; M-1 to R-86; M-1 to I-85; M-1 to A-84; M-1 to Q-83; M-1 to R-82; M-1 to A-81; M-1 to K-80; M-1 to V-79; M-1 to D-78; M-1 to S-77; M-1 to P-76; M-1 to H-75; M-1 to S-74; M-1 to T-73; M-1 to V-72; M-1 to L-71; M-1 to I-70; M-1 to V-69; M-1 to L-68; M-1 to F-67; M-1 to P-66; M-1 to N-65; M-I to Q-64; M-I to H-63; M-1 to S-62; M-1 to C-61; M-1 to N-60; M-1 to S-59; M-1 to H-58; M-1 to E-57; M-1 to R-56; M-1 to L-55; M-1 to T-54; M-1 to F-53; M-1 to H-52; M-1 to F-51; M-1 to D-50; M-1 to Q-49; M-1 to R-48; M-1 to Y-47; M-1 to I-46; M-1 to P-45; M-1 to E-44; M-1 to Y-43; M-1 to E-42; M-1 to Y-41; M-1 to F-40; M-1 to Y-39; M-1 to M-38; M-1 to W-37; M-1 to N-36; M-1 to V-35; M-1 to R-34; M-1 to E-33; M-1 to 1-32; M-1 to V-31; M-1 to N-30; M-1 to Y-29; M-1 to H-28; M-1 to P-27; M-1 to L-26; M-1 to S-25; M-1 to L-24; M-1 to Y-23; M-1 to W-22; M-1 to M-21; M-1 to V-20; M-1 to F-19; M-1 to F-18; M-1 to S-17; M-1 to L-16; M-1 to L-15; M-1 to S-14; M-1 to L-13; M-1 to L-12; M-1 to L-11; M-1 to L-10; M-1 to S-9; M-1 to W-8; M-1 to K-7; and M-1 to L-6 of the sequence of the Dendriac sequence shown in FIGS. 1A, 1B, and 1C (which is identical to the sequence shown as SEQ ID NO:2 and as SEQ ID NO: 10, with the exception that the amino acid residues in FIGS. 1A, 1B, and 1C are numbered consecutively from 1 through 319 from the N-terminus to the C-terminus, while the amino acid residues in SEQ ID NO:2 and in SEQ ID NO: 10 are numbered consecutively from −25 through 294 to reflect the position of the predicted signal peptide). Polynucleotides encoding these polypeptides also are provided.

The invention also provides polypeptides having one or more amino acids deleted from both the amino and the carboxyl termini of a Dendriac polypeptide, which may be described generally as having residues n³-m³ of FIGS. 1A, 1B, and 1C (i.e., SEQ ID NO:2 and SEQ ID NO:10), where n³ and m³ are integers as described above.

Again as mentioned above, even if deletion of one or more amino acids from the N-terminus of a protein results in modification of loss of one or more biological functions of the protein, other biological activities may still be retained. Thus, the ability of the shortened Brainiac-3 mutein to induce and/or bind to antibodies which recognize the complete or mature form of the polypeptide generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a Brainiac-3 mutein with a large number of deleted N-terminal amino acid residues may retain some biological or immunogenic activities. In fact, peptides composed of as few as six Brainiac-3 amino acid residues may often evoke an immune response.

Accordingly, the present invention further provides polypeptides having one or more residues deleted from the amino terminus of the Brainiac-3 amino acid sequence shown in FIGS. 2A and 2B (SEQ ID NO:4 and in SEQ ID NO:12), up to the glycine residue at position number 347 and polynucleotides encoding such polypeptides. In particular, the present invention provides polypeptides comprising the amino acid sequence of residues n⁴-352 of FIGS. 2A and B (SEQ ID NO:4 and SEQ ID NO:12), where n⁴ is an integer in the range of 2 to 347, and 348 is the position of the first residue from the N-terminus of the complete Brainiac-3 polypeptide believed to be required for at least immunogenic activity of the Brainiac-3 polypeptide.

More in particular, the invention provides polynucleotides encoding polypeptides comprising, or alternatively consisting of, the amino acid sequence of residues of L-2 to R-352; C-3 to R-352; R-4 to R-352; L-5 to R-352; C-6 to R-352; W-7 to R-352; L-8 to R-352; V-9 to R-352; S-10 to R-352; Y-11 to R-352; S-12 to R-352; L-13 to R-352; A-14 to R-352; V-15 to R-352; L-16 to R-352; L-17 to R-352; L-18 to R-352; G-19 to R-352; C-20 to R-352; L-21 to R-352; L-22 to R-352; F-23 to R-352; L-24 to R-352; R-25 to R-352; K-26 to R-352; A-27 to R-352; A-28 to R-352; K-29 to R-352; P-30 to R-352; A-31 to R-352; E-32 to R-352; T-33 to R-352; P-34 to R-352; R-35 to R-352; P-36 to R-352; T-37 to R-352; S-38 to R-352; L-39 to R-352; S-40 to R-352; G-41 to R-352; A-42 to R-352; P-43 to R-352; P-44 to R-352; T-45 to R-352; P-46 to R-352; R-47 to R-352; H-48 to R-352; S-49 to R-352; R-50 to R-352; C-51 to R-352; P-52 to R-352; P-53 to R-352; N-54 to R-352; H-55 to R-352; T-56 to R-352; V-57 to R-352; S-58 to R-352; S-59 to R-352; A-60 to R-352; S-61 to R-352; L-62 to R-352; S-63 to R-352; L-64 to R-352; P-65 to R-352; S-66 to R-352; R-67 to R-352; H-68 to R-352; R-69 to R-352; L-70 to R-352; F-71 to R-352; L-72 to R-352; T-73 to R-352; Y-74 to R-352; R-75 to R-352; H-76 to R-352; C-77 to R-352; R-78 to R-352; N-79 to R-352; F-80 to R-352; S-81 to R-352; 1-82 to R-352; L-83 to R-352; L-84 to R-352; E-85 to R-352; P-86 to R-352; S-87 to R-352; G-88 to R-352; C-89 to R-352; S-90 to R-352; K-91 to R-352; D-92 to R-352; T-93 to R-352; F-94 to R-352; L-95 to R-352; L-96 to R-352; L-97 to R-352; A-98 to R-352; I-99 to R-352; K-100 to R-352; S-101 to R-352; Q-102 to R-352; P-103 to R-352; G-104 to R-352; H-105 to R-352; V-106 to R-352; E-107 to R-352; R-108 to R-352; R-109 to R-352; A-i 10 to R-352; A-111 to R-352; I-112 to R-352; R-113 to R-352; S-114 to R-352; T-115 to R-352; W-1 16 to R-352; G-1 17 to R-352; R-1 18 to R-352; W-l 19 to R-352; G-120 to R-352; D-121 to R-352; G-122 to R-352; L-123 to R-352; G-124 to R-352; P-125 to R-352; A-126 to R-352; L-127 to R-352; K-128 to R-352; L-129 to R-352; V-130 to R-352; F-131 to R-352; L-132 to R-352; L-133 to R-352; G-134 to R-352; V-135 to R-352; A-136 to R-352; G-137 to R-352; S-1138 to R-352; A-139 to R-352; P-140 to R-352; P-141 to R-352; A-142 to R-352; Q-143 to R-352; L-144 to R-352; L-145 to R-352; A-146 to R-352; Y-147 to R-352; E-148 to R-352; S-149 to R-352; R-150 to R-352; E-151 to R-352; F-i52 to R-352; D-153 to R-352; D-154 to R-352; 1-155 to R-352; L-156 to R-352; Q-157 to R-352; W-158 to R-352; D-159 to R-352; F-160 to R-352; T-161 to R-352; E-162 to R-352; D-163 to R-352; F-164 to R-352; F-165 to R-352; N-166 to R-352; L-167 to R-352; T-168 to R-352; L-169 to R-352; K-170 to R-352; E-171 to R-352; L-172 to R-352; H-173 to R-352; L-174 to R-352; Q-175 to R-352; R-176 to R-352; W-177 to R-352; V-178 to R-352; V-179 to R-352; A-180 to R-352; A-181 to R-352; C-182 to R-352; P-183 to R-352; Q-184 to R-352; A -185 to R-352; H-186 to R-352; F-187 to R-352; M-188 to R-352; L-189 to R-352; K-190 to R-352; G-191 to R-352; D-192 to R-352; D-193 to R-352; D-194 to R-352; V-195 to R-352; F-196 to R-352; V-197 to R-352; H-198 to R-352; V-199 to R-352; P-200 to R-352; N-201 to R-352; V-202 to R-352; L-203 to R-352; E-204 to R-352; F-205 to R-352; L-206 to R-352; D-207 to R-352; G-208 to R-352; W-209 to R-352; D-210 to R-352; P-211 to R-352; A-212 to R-352; Q-213 to R-352; D-214 to R-352; L-215 to R-352; L-216 to R-352; V-217 to R-352; G-218 to R-352; D-219 to R-352; V-220 to R-352; I-221 to R-352; R-222 to R-352; Q-223 to R-352; A-224 to R-352; L-225 to R-352; P-226 to R-352; N-227 to R-352; R-228 to R-352; N-229 to R-352; T-230 to R-352; K-231 to R-352; V-232 to R-352; K-233 to R-352; Y-234 to R-352; F-235 to R-352; 1-236 to R-352; P-237 to R-352; P-238 to R-352; S-239 to R-352; M-240 to R-352; Y-241 to R-352; R-242 to R-352; A-243 to R-352; T-244 to R-352; H-245 to R-352; Y-246 to R-352; P-247 to R-352; P-248 to R-352; Y-249 to R-352; A-250 to R-352; G-251 to R-352; G-252 to R-352; G-253 to R-352; G-254 to R-352; Y-255 to R-352; V-256 to R-352; M-257 to R-352; S-258 to R-352; R-259 to R-352; A-260 to R-352; T-261 to R-352; V-262 to R-352; R-263 to R-352; R-264 to R-352; L-265 to R-352; Q-266 to R-352; A-267 to R-352; 1-268 to R-352; M-269 to R-352; E-270 to R-352; D-271 to R-352; A-272 to R-352; E-273 to R-352; L-274 to R-352; F-275 to R-352; P-276 to R-352; I-277 to R-352; D-278 to R-352; D-279 to R-352; V-280 to R-352; F-281 to R-352; V-282 to R-352; G-283 to R-352; M-284 to R-352; C-285 to R-352; L-286 to R-352; R-287 to R-352; R-288 to R-352; L-289 to R-352; G-290 to R-352; L-291 to R-352; S-292 to R-352; P-293 to R-352; M-294 to R-352; H-295 to R-352; H-296 to R-352; A-297 to R-352; G-298 to R-352; F-299 to R-352; K-300 to R-352; T-301 to R-352; F-302 to R-352; G-303 to R-352; 1-304 to R-352; R-305 to R-352; R-306 to R-352; P-307 to R-352; L-308 to R-352; D-309 to R-352; P-310 to R-352; L-311 to R-352; D-312 to R-352; P-313 to R-352; C-314 to R-352; L-315 to R-352; Y-316 to R-352; R-317 to R-352; G-318 to R-352; L-319 to R-352; L-320 to R-352; L-321 to R-352; V-322 to R-352; H-323 to R-352; R-324 to R-352; L-325 to R-352; S-326 to R-352; P-327 to R-352; L-328 to R-352; E-329 to R-352; M-330 to R-352; W-331 to R-352; T-332 to R-352; M-333 to R-352; W-334 to R-352; A-335 to R-352; L-336 to R-352; V-337 to R-352; T-338 to R-352; D-339 to R-352; E-340 to R-352; G-341 to R-352; L-342 to R-352; K-343 to R-352; C-344 to R-352; A-345 to R-352; A-346 to R-352; and G-347 to R-352 of the Brainiac-3 sequence shown in FIGS. 2A and 2B (which is identical to the sequence shown as SEQ ID NO:4 and in SEQ ID NO: 12, with the exception that the amino acid residues in FIGS. 2A and 2B are numbered consecutively from 1 through 352 from the N-terminus to the C-terminus, while the amino acid residues in SEQ ID NO:4 and in SEQ ID NO: 12 are numbered consecutively from −28 through 324 to reflect the position of the predicted signal peptide). Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened Brainiac-3 mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a Brainiac-3 mutein with a large number of deleted C-terminal amino acid residues may retain some biological or immunogenic activities. In fact, peptides composed of as few as six Brainiac-3 amino acid residues may often evoke an immune response.

Accordingly, the present invention further provides polypeptides having one or more residues deleted from the carboxy terminus of the amino acid sequence of the Brainiac-3 shown in FIGS. 2A and 2B (i.e., SEQ ID NO:4 and SEQ ID NO: 12), up to the cysteine residue at position number 6, and polynucleotides encoding such polypeptides. In particular, the present invention provides polypeptides comprising the amino acid sequence of residues 1-m⁴ of FIGS. 2A and 2B (i.e., SEQ ID NO:4 and SEQ ID NO: 12), where m⁴ is an integer in the range of 6 to 351, and 6 is the position of the first residue from the C-terminus of the complete Brainiac-3 polypeptide believed to be required for at least immunogenic activity of the Brainiac-3 polypeptide.

More in particular, the invention provides polynucleotides encoding polypeptides comprising, or alternatively consisting of, the amino acid sequence of residues M-1 to Q-351; M-1 to P-350; M-1 to I-349; M-1 to P-348; M-1 to G-347; M-1 to A-346; M-1 to A-345; M-1 to C-344; M-1 to K-343; M-1 to L-342; M-1 to G-341; M-1 to E-340; M-1 to D-339; M-1 to T-338; M-1 to V-337; M-1 to L-336; M-1 to A-335; M-1 to W-334; M-1 to M-333; M-1 to T-332; M-1 to W-331; M-1 to M-330; M-1 to E-329; M-1 to L-328; M-1 to P-327; M-1 to S-326; M-1 to L-325; M-1 to R-324; M-1 to H-323; M-1 to V-322; M-1 to L-321; M-1 to L-320; M-1 to L-319; M-1 to G-318; M-1 to R-317; M-1 to Y-316; M-1 to L-315; M-1 to C-314; M-1 to P-313; M-1 to D-312; M-1 to L-311; M-1 to P-310; M-1 to D-309; M-1 to L-308; M-1 to P-307; M-1 to R-306; M-1 to R-305; M-1 to I-304; M-1 to G-303; M-1 to F-302; M-1 to T-301; M-1 to K-300; M-1 to F-299; M-1 to G-298; M-1 to A-297; M-1 to H-296; M-1 to H-295; M-1 to M-294; M-1 to P-293; M-1 to S-292; M-1 to L-291; M-1 to G-290; M-1 to L-289; M-1 to R-288; M-1 to R-287; M-1 to L-286; M-1 to C-285; M-1 to M-284; M-1 to G-283; M-1 to V-282; M-1 to F-281; M-1 to V-280; M-1 to D-279; M-1 to D-278; M-1 to I-277; M-1 to P-276; M-1 to F-275; M-1 to L-274; M-1 to E-273; M-1 to A-272; M-1 to D-271; M-1 to E-270; M-1 to M-269; M-1 to I-268; M-1 to A-267; M-1 to Q-266; M-1 to L-265; M-1 to R-264; M-1 to R-263; M-1 to V-262; M-1 to T-261; M-1 to A-260; M-1 to R-259; M-1 to S-258; M-1 to M-257; M-1 to V-256; M-1 to Y-255; M-1 to G-254; M-1 to G-253; M-1 to G-252; M-1 to G-251; M-1 to A-250; M-1 to Y-249; M-1 to P-248; M-1 to P-247; M-1 to Y-246; M-1 to H-245; M-1 to T-244; M-1 to A-243; M-1 to R-242; M-1 to Y-241; M-1 to M-240; M-1 to S-239; M-1 to P-238; M-1 to P-237; M-1 to I-236; M-1 to F-235; M-1 to Y-234; M-1 to K-233; M-1 to V-232; M-1 to K-231; M-1 to T-230; M-1 to N-229; M-1 to R-228; M-1 to N-227; M-1 to P-226; M-1 to L-225; M-1 to A-224; M-1 to Q-223; M-1 to R-222; M-1 to I-221; M-1 to V-220; M-1 to D-219; M-1 to G-218; M-1 to V-217; M-1 to L-216; M-1 to L-215; M-1 to D-214; M-1 to Q-213; M-1 to A-212; M-1 to P-21 1; M-1 to D-210; M-I to W-209; M-1 to G-208; M-1 to D-207; M-1 to L-206; M-1 to F-205; M-1 to E-204; M-1 to L-203; M-1 to V-202; M-1 to N-201; M-1 to P-200; M-1 to V-199; M-1 to H-198; M-1 to V-197; M-1 to F-196; M-1 to V-195; M-1 to D-194; M-1 to D-193; M-1 to D-192; M-1 to G-191; M-1 to K-190; M-1 to L-189; M-1 to M-188; M-1 to F-187; M-1 to H-186; M-1 to A-185; M-1 to Q-184; M-1 to P-183; M-1 to C-182; M-1 to A-181; M-1 to A-180; M-1 to V-179; M-1 to V-178; M-1 to W-177; M-1 to R-176; M-1 to Q-175; M-1 to L-174; M-1 to H-173; M-1 to L-172; M-1 to E-171; M-1 to K-170; M-1 to L-169; M-1 to T-168; M-1 to L-167; M-1 to N-166; M-1 to F-165; M-1 to F-164; M-1 to D-163; M-1 to E-162; M-1 to T-161; M-1 to F-160; M-1 to D-159; M-1 to W-158; M-1 to Q-157; M-1 to L-156; M-1 to I-155; M-1 to D-154; M-1 to D-153; M-1 to F-152; M-1 to E-151; M-1 to R-150; M-1 to S-149; M-1 to E-148; M-1 to Y-147; M-1 to A-146; M-1 to L-145; M-1 to L-144; M-1 to Q-143; M-1 to A-142; M-1 to P-141; M-1 to P-140; M-1 to A-139; M-1 to S-138; M-1 to G-137; M-1 to A-136; M-1 to V-135; M-1 to G-134; M-1 to L-133; M-1 to L-132; M-1 to F-131; M-1 to V-130; M-1 to L-129; M-1 to K-128; M-1 to L-127; M-1 to A-126; M-1 to P-125; M-1 to G-124; M-1 to L-123; M-1 to G-122; M-1 to D-121; M-1 to G-120; M-1 to W-119; M-1 to R-118; M-1 to G-117; M-1 to W-116; M-1 to T-115; M-1 to S-114; M-1 to R-113; M-1 to I-112; M-1 to A-111; M-1 to A-110; M-1 to R-109; M-1 to R-108; M-1 to E-107; M-1 to V-106; M-1 to H-105; M-1 to G-104-M-1 to P-103; M-1 to Q-102; M-1 to S-101; M-1 to K-100; M-1 to 1-99; M-1 to A-98; M-1 to L-97; M-1 to L-96; M-1 to L-95; M-1 to F-94; M-1 to T-93; M-1 to D-92; M-1 to K-91; M-1 to S-90; M-1 to C-89; M-1 to G-88; M-1 to S-87; M-1 to P-86; M-1 to E-85; M-1 to L-84; M-1 to L-83; M-1 to I-82; M-1 to S-81; M-1 to F-80; M-1 to N-79; M-1 to R-78; M-1 to C-77; M-1 to H-76; M-1 to R-75; M-1 to Y-74; M-1 to T-73; M-1 to L-72; M-1 to F-71; M-1 to L-70; M-1 to R-69; M-1 to H-68; M-1 to R-67; M-1 to S-66; M-1 to P-65; M-1 to L-64; M-1 to S-63; M-1 to L-62; M-1 to S-61; M-1 to A-60; M-1 to S-59; M-1 to S-58; M-1 to V-57; M-1 to T-56; M-1 to H-55; M-1 to N-54; M-1 to P-53; M-1 to P-52; M-1 to C-51; M-1 to R-50; M-1 to S-49; M-1 to H-48; M-1 to R-47; M-1 to P-46; M-1 to T-45; M-1 to P-44; M-1 to P-43; M-1 to A-42; M-1 to G-41; M-1 to S-40; M-1 to L-39; M-1 to S-38; M-1 to T-37; M-1 to P-36; M-1 to R-35; M-1 to P-34; M-1 to T-33; M-1 to E-32; M-1 to A-31; M-1 to P-30; M-1 to K-29; M-1 to A-28; M-1 to A-27; M-1 to K-26; M-1 to R-25; M-1 to L-24; M-1 to F-23; M-1 to L-22; M-1 to L-21; M-1 to C-20; M-1 to G-19; M-1 to L-18; M-1 to L-17; M-1 to L-16; M-1 to V-15; M-1 to A-14; M-1 to L-13; M-1 to S-12; M-1 to Y-11; M-1 to S-10; M-1 to V-9; M-1 to L-8; M-1 to W-7; and M-1 to C-6 of the sequence of the Brainiac-3 sequence shown in FIGS. 2A and 2B (which is identical to the sequence shown as SEQ ID NO:4 and as SEQ ID NO: 12, with the exception that the amino acid residues in FIGS. 2A and 2B are numbered consecutively from 1 through 352 from the N-terminus to the C-terminus, while the amino acid residues in SEQ ID NO:4 and in SEQ ID NO:12 are numbered consecutively from −28 through 324 to reflect the position of the predicted signal peptide). Polynucleotides encoding these polypeptides also are provided.

The invention also provides polypeptides having one or more amino acids deleted from both the amino and the carboxyl termini of a Brainiac-3 polypeptide, which may be described generally as having residues n⁴-m⁴ of FIGS. 2A and B (SEQ ID NO:4 and SEQ ID NO:12), where n⁴ and m⁴ are integers as described above.

Other Mutants

In addition to terminal deletion forms of the protein discussed above, it also will be recognized by one of ordinary skill in the art that some amino acid sequences of the Dendriac and Brainiac-3 polypeptides can be varied without significant effect of the structure or function of the protein. If such differences in sequence are contemplated, it should be remembered that there will be critical areas on the protein which determine activity.

Thus, the invention further includes variations of the Dendriac and Brainiac-3 polypeptides which show substantial Dendriac or Brainiac-3 polypeptide activity or which include regions of Dendriac and Brainiac-3 polypeptides such as the polypeptide portions discussed below. Such mutants include deletions, insertions, inversions, repeats, and type substitutions selected according to general rules known in the art so as have little effect on activity. For example, guidance concerning how to make phenotypically silent amino acid substitutions is provided wherein the authors indicate that there are two main approaches for studying the tolerance of an amino acid sequence to change (Bowie, J. U., et al, Science 247:1 306-1310 (1990)),. The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described by Bowie and coworkers (supra) and the references cited therein. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptide of SEQ ID NO:2, SEQ ID NO:4, SEQ ID NO:10, SEQ ID NO:12, or those encoded by the deposited cDNAs, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which either the Dendriac or Brainiac-3 mature polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein.

Thus, the Dendriac and Brainiac-3 polypeptides of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table III).

TABLE III
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the Dendriac and/or Brainiac-3 polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard, et al., Clin. Exp. Immunol. 2:331-340 (1967); Robbins, et al., Diabetes 36:838-845 (1987); Cleland, et al., Crit. Rev. Therapeutic Drug Carrier Systems 10:307-377 (1993)).

Replacement of amino acids can also change the selectivity of the binding of a ligand to cell surface receptors (for example, Ostade, et al., Nature 361:266-268 (1993)) describes certain mutations resulting in selective binding of TNF-α to only one of the two known types of TNF receptors. Sites that are critical for ligand-receptor binding can also be determined by structural analysis such as crystallization, nuclear magnetic resonance or photoaffinity labeling (Smith, et al., J. Mol. Biol. 224:899-904 (1992); de Vos, et al. Science 255:306-312 (1992)).

The polypeptides of the present invention are preferably provided in an isolated form, and preferably are substantially purified. Recombinantly produced versions of the Dendriac and/or Brainiac-3 polypeptides can be substantially purified by the one-step method described by Smith and Johnson (Gene 67:31-40 (1988)). Polypeptides of the invention also can be purified from natural or recombinant sources using anti-Dendriac and/or anti-Brainiac-3 antibodies of the invention in methods which are well known in the art of protein purification.

The invention also provides an isolated Dendriac polypeptide comprising an amino acid sequence selected from the group consisting of: (a) the amino acid sequence of the full-length Dendriac polypeptide having the complete amino acid sequence shown in SEQ ID NO:2 and SEQ ID NO: 10 (i.e., positions 1 -319 of SEQ ID NO:2 and SEQ ID NO: 10); (b) the amino acid sequence of the full-length Dendriac polypeptide having the complete amino acid sequence shown in SEQ ID NO:2 and SEQ ID NO:10 excepting the N-terminal methionine (i.e., positions 2-319 of SEQ ID NO:2 and SEQ ID NO: 10); (c) the amino acid sequence of the predicted mature Dendriac polypeptide having the amino acid sequence at positions 26-319 in SEQ ID NO:2 and SEQ ID NO:10; (d) the complete amino acid sequence encoded by the cDNA clone contained in the ATCC Deposit No. 203056 and in ATCC Deposit No. 209627; (e) the complete amino acid sequence excepting the N-terminal methionine encoded by the cDNA clone contained in the ATCC Deposit No. 203056 and in ATCC Deposit No. 209627; and (f) the complete amino acid sequence of the predicted mature Dendriac polypeptide encoded by the cDNA clone contained in the ATCC Deposit No. 203056 and in ATCC Deposit No. 209627. The polypeptides of the present invention also include polypeptides having an amino acid sequence at least 80% identical, more preferably at least 90% identical, and still more preferably 95%, 96%, 97%, 98% or 99% identical to those described in (a), (b), (c), (d), (e) or (f), above, as well as polypeptides having an amino acid sequence with at least 90% similarity, and more preferably at least 95% similarity, to those above.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNA or to the polypeptide of SEQ ID NO:2 and SEQ ID NO: 10, and also include portions of such polypeptides with at least 15 amino acids, more preferably at least 30 amino acids, even more preferably at least 40 amino acids, still even more preferably at least 50 amino acids, still more preferably at least 60 amino acids, and yet even more preferably at least 75 amino acids.

In addition, the invention provides an isolated Brainiac-3 polypeptide comprising an amino acid sequence selected from the group consisting of: (a) the amino acid sequence of the full-length Brainiac-3 polypeptide having the complete amino acid sequence shown in SEQ ID NO:4 and SEQ ID NO:12 (i.e., positions 1-352 of SEQ ID NO:4 and SEQ ID NO:12); (b) the amino acid sequence of the fill-length Brainiac-3 polypeptide having the complete amino acid sequence shown in SEQ ID NO:4 and SEQ ID NO:12 excepting the N-terminal methionine (i.e., positions 2-352 of SEQ ID NO:4 and SEQ ID NO: 12); (c) the amino acid sequence of the predicted mature Brainiac-3 lo polypeptide having the amino acid sequence at positions 29-352 in SEQ ID NO:4 and SEQ ID NO:12; (d) the complete amino acid sequence encoded by the cDNA clone contained in the ATCC Deposit No. 203451 and in ATCC Deposit No. 209463; (e) the complete amino acid sequence excepting the N-terminal methionine encoded by the cDNA clone contained in the ATCC Deposit No. 203451 and in ATCC Deposit No. 209463; and (f) the complete amino acid sequence of the predicted mature Brainiac-3 polypeptide encoded by the cDNA clone contained in the ATCC Deposit No. 203451 and in ATCC Deposit No. 209463. The polypeptides of the present invention also include polypeptides having an amino acid sequence at least 80% identical, more preferably at least 90% identical, and still more preferably 95%, 96%, 97%, 98% or 99% identical to those described in (a), (b), (c), (d), (e) or (f), above, as well as polypeptides having an amino acid sequence with at least 90% similarity, and more preferably at least 95% similarity, to those above.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNA or to the polypeptide of SEQ ID NO:4 and SEQ ID NO: 12, and also include portions of such polypeptides with at least 15 amino acids, more preferably at least 30 amino acids, even more preferably at least 40 amino acids, still even more preferably at least 50 amino acids, still more preferably at least 60 amino acids, and yet even more preferably at least 75 amino acids.

A further embodiment of the invention relates to a peptide or polypeptide which comprises the amino acid sequence of a Dendriac or Brainiac-3 polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a peptide or polypeptide to have an amino acid sequence which comprises the amino acid sequence of a Dendriac or Brainiac-3 polypeptide, which contains at least one, but not more than 10, 9, 8, 7, 6, 5, 4, 3, 2 or 1 conservative amino acid substitutions.

Representative examples of polypeptide fragments of the invention, include, for example, fragments from about amino acid number 1-20, 21-40, 41-60, 61-80, 81-100, 101-120, 121-140, 141-160, or 161 to the end of the coding region of SEQ ID NO:2, SEQ ID NO:4, SEQ ID NO: 10, SEQ ID NO: 12 or to a polypeptide expressed from any of the deposited cDNA clones which express Dendriac or Brainiac-3. Moreover, polypeptide fragments can be about 20, 30, 40, 50, 60, 70, 80, 90, 100, 110, 120, 130, 140, or 150 amino acids in length. In this context about includes the particularly recited ranges, larger or smaller by several (5, 4, 3, 2, or 1) amino acids, at either extreme or at both extremes. The invention also provides an isolated polypeptide comprising an amino acid sequence at least 90% identical to a sequence of at least about 10, 30 or 100 contiguous amino acids in the amino acid sequence of SEQ ID NO:2, SEQ ID NO:4, SEQ ID NO:10 or SEQ ID NO: 12.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

By a polypeptide having an amino acid sequence at least, for example, 95% “identical” to a reference amino acid sequence of a Dendriac or Brainiac-3 polypeptide is intended that the amino acid sequence of the polypeptide is identical to the reference sequence except that the polypeptide sequence may include up to five amino acid alterations per each 100 amino acids of the reference amino acid of the Dendriac or Brainiac-3 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIGS. 1A, 1B, and 1C (SEQ ID NO:2) and SEQ ID NO:10, the amino acid sequence shown in FIGS. 2A and 2B (SEQ ID NO:4) and SEQ ID NO:12, the amino acid sequence encoded by deposited cDNA clones HFVIF40 and HFCCQ50, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting Dendriac and/or Brainiac-3 polypeptide expression as described below or as agonists and antagonists capable of enhancing or inhibiting Dendriac and/or Brainiac-3 polypeptide function. Further, such polypeptides can be used in the yeast two-hybrid system to “capture” Dendriac and/or Brainiac-3 polypeptide-binding polypeptides which are also candidate agonists and antagonists according to the present invention. The yeast two hybrid system is described by Fields and Song (Nature 340:245-246 (1989)).

Epitope-Bearing Portions

In another embodiment, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a polypeptide that elicits an antibody response when the complete or whole polypeptide is the immunogen. On the other hand, a region of a protein molecule to which an antibody can bind is defined as an “antigenic epitope.” The number of immunogenic epitopes of a protein generally is less than the number of antigenic epitopes (see, for instance, Geysen, et al., Proc. Natl. Acad. Sci. USA 81:3998-4002 (1983)).

As to the selection of peptides or polypeptides bearing an antigenic epitope (i.e., that contain a region of a polypeptide molecule to which an antibody can bind), it is well known in that art that relatively short synthetic peptides that mimic part of a polypeptide sequence are routinely capable of eliciting an antiserum that reacts with the partially mimicked polypeptide (see, for instance, Sutcliffe, J. G., et al., Science 219:660-666 (1983)). Peptides capable of eliciting protein-reactive sera are frequently represented in the primary sequence of a polypeptide, can be characterized by a set of simple chemical rules, and are confined neither to immunodominant regions of intact polypeptides (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention (see, for instance, Wilson, et al., Cell 37:767-778 (1984)).

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate Dendriac-specific antibodies include: a polypeptide comprising amino acid residues from about Leu-30 to about His-38 in SEQ ID NO:2 and SEQ ID NO:10; from about His-50 to about Ala-59 in SEQ ID NO:2 and SEQ ID NO:10; from about Trp-64 to about Trp-70 in SEQ ID NO:2 and SEQ ID NO:10; from about Met-208 to about Val-213 in SEQ ID NO:2 and SEQ ID NO:10; from about Lys-224 to about Asp-230 in SEQ ID NO:2 and SEQ ID NO:10; from about Ile-246 to about LeuA-52 in SEQ ID NO:2 and SEQ ID NO:10; and from about Gly-273 to about Glu-278 in SEQ ID NO:2 and SEQ ID NO: 10. These polypeptide fragments have been determined to bear antigenic epitopes of the Dendriac polypeptide by the analysis of the Jameson-Wolf antigenic index, as shown in FIG. 4 and Table I, above.

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate Brainiac-3-specific antibodies include: a polypeptide comprising amino acid residues from about Lys-l to about Ser-12 in SEQ ID NO:4 and SEQ ID NO: 12; from about Pro-16 to about Pro-25 in SEQ ID NO:4 and SEQ ID NO:12; from about Leu-36 to about Arg-41 in SEQ ID NO:4 and SEQ ID NO:12; from about Pro-58 to about Asp-64 in SEQ ID NO:4 and SEQ ID NO:12; from about Ser-73 to about Ile-84 in SEQ ID NO:4 and SEQ ID NO:12; from about Thr-87 to about Pro-97 in SEQ ID NO:4 and SEQ ID NO:12; from about Leu-161 to about Val-167 in SEQ ID NO:4 and SEQ ID NO:12; from about Asp-179 to about Gln-185 in SEQ ID NO:4 and SEQ ID NO:12; from about Leu-197 to about Lys-203 in SEQ ID NO:4 and SEQ ID NO:12; from about Gly-275 to about Asp-284 in SEQ ID NO:4 and SEQ ID NO:12; and from about Arg-296 to about Glu-301 in SEQ ID NO:4 and SEQ ID NO:12. These polypeptide fragments have been determined to bear antigenic epitopes of the Brainiac-3 polypeptide by the analysis of the Jameson-Wolf antigenic index, as shown in FIG. 5 and Table II, above.

The epitope-bearing peptides and polypeptides of the invention may be produced by any conventional means (see, for example, Houghten, R. A., et al, Proc. Natl. Acad. Sci. USA 82:5131-5135 (1985); and U.S. Pat. No. 4,631,211 to Houghten, et al. (1986)).

Epitope-bearing peptides and polypeptides of the invention are used to induce antibodies according to methods well known in the art (see, for instance, Sutcliffe, et al, supra; Wilson, et al., supra; Chow, M., et al., Proc. Natl. Acad. Sci. USA 82:910-914; and Bittle, F. J., et al., J. Gen. Virol. 66:2347-2354 (1985)). Immunogenic epitope-bearing peptides of the invention, i.e., those parts of a protein that elicit an antibody response when the whole protein is the immunogen, are identified according to methods known in the art (see, for instance, Geysen, et al., supra). Further still, U.S. Pat. No. 5,194,392, issued to Geysen, describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092, issued to Geysen, describes a method of detecting or determining a sequence of monomers which is a topographical equivalent of a ligand which is complementary to the ligand binding site of a particular receptor of interest. Similarly, U.S. Pat. No. 5,480,971, issued to Houghten and colleagues, on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Fusion Proteins

As one of skill in the art will appreciate, Dendriac and/or Brainiac-3 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker, et al., Nature 331:84-86 (1988)). Fusion proteins that have a disulfide-linked dimeric structure due to the IgG part can also be more efficient in binding and neutralizing other molecules than the monomeric Dendriac and/or Brainiac-3 polypeptide or polypeptide fragment alone (Fountoulakis, et al., J. Biochem. 270:3958-3964 (1995)).

Antibodies

Dendriac and/or Brainiac-3 polypeptide-specific antibodies for use in the present invention can be raised against the intact Dendriac and/or Brainiac-3 polypeptide or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)2 fragments) which are capable of specifically binding to Dendriac and/or Brainiac-3 polypeptides. Fab and F(ab′)2 fragments lack the Fc fragment of intact antibody, clear more rapidly from the circulation, and may have less non-specific tissue binding of an intact antibody (Wahl, et al., J. Nucl. Med. 24:316-325 (1983)). Thus, these fragments are preferred.

The antibodies of the present invention may be prepared by any of a variety of methods. For example, cells expressing the Dendriac and/or Brainiac-3 polypeptides or an antigenic fragment thereof can be administered to an animal in order to induce the production of sera containing polyclonal antibodies. In a preferred method, a preparation of Dendriac and/or Brainiac-3 polypeptide is prepared and purified to render it substantially free of natural contaminants. Such a preparation is then introduced into an animal in order to produce polyclonal antisera of greater specific activity.

In the most preferred method, the antibodies of the present invention are monoclonal antibodies (or Dendriac and/or Brainiac-3 polypeptide-binding fragments thereof). Such monoclonal antibodies can be prepared using hybridoma technology (Kohler, et al, Nature 256:495 (1975); Kohler, et al., Eur. J Immunol. 6:511 (1976); Kohler, et al., Eur. J. Immunol. 6:292 (1976); Hammerling, et al., in: Monoclonal Antibodies and T-Cell Hybridomas, Elsevier, N.Y., (1981) pp. 563-681)). In general, such procedures involve immunizing an animal (preferably a mouse) with a Dendriac and/or Brainiac-3 polypeptide antigen or, more preferably, with a Dendriac and/or Brainiac-3 polypeptide-expressing cell. Suitable cells can be recognized by their capacity to bind anti-Dendriac and/or Brainiac-3 polypeptide antibody. Such cells may be cultured in any suitable tissue culture medium; however, it is preferable to culture cells in Earles modified Eagles medium supplemented with 10% fetal bovine serum (inactivated at about 56° C.), and supplemented with about 10 μg/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, Rockville, Md. After fusion, the resulting hybridoma cells are selectively maintained in HAT medium, and then cloned by limiting dilution as described by Wands and colleagues (Gastroenterology 80:225-232 (1981)). The hybridoma cells obtained through such a selection are then assayed to identify clones which secrete antibodies capable of binding the Dendriac and/or Brainiac-3 polypeptide antigen.

Alternatively, additional antibodies capable of binding to the Dendriac and/or Brainiac-3 polypeptide antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, Dendriac and/or Brainiac-3 polypeptide-specific antibodies are used to immunize an animal, preferably a mouse. The splenocytes of such an animal are then used to produce hybridoma cells, and the hybridoma cells are screened to identify clones which produce an antibody whose ability to bind to the Dendriac and/or Brainiac-3 polypeptide-specific antibody can be blocked by the Dendriac and/or Brainiac-3 polypeptide antigen. Such antibodies comprise anti-idiotypic antibodies to the Dendriac and/or Brainiac-3 polypeptide-specific antibody and can be used to immunize an animal to induce formation of further Dendriac and/or Brainiac-3 polypeptide-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, Dendriac and/or Brainiac-3 polypeptide-binding fragments can be produced through the application of recombinant DNA technology or through synthetic chemistry.

For in vivo use of anti-Dendriac and/or Brainiac-3 in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (Morrison, Science 229:1202 (1985); Oi, et al., BioTechniques 4:214 (1986); Cabilly, et al., U.S. Pat. No. 4,816,567; Taniguchi, et al., EP 171496; Morrison, et al., EP 173494; Neuberger, et al., WO 8601533; Robinson, et al., WO 8702671; Boulianne, et al., Nature 312:643 (1984); Neuberger, et al, Nature 314:268 (1985).

### Disorders Related to the Immune and Nervous Systems

Diagnosis

The present inventors have discovered that Dendriac is expressed not only in dendritic cells, but also (using BLAST analysis of the HGS EST database) in NTERA2 cells, adult pulmonary tissue, salivary gland, ovary, Caco-2 colon adenocarcinoma cell line, smooth muscle, cerebellum, 8 week old whole human embryo, hemagiopericytoma, amygdala, substantia nigra, and whole brain. In addition, the present inventors have discovered that Brainiac-3 is expressed not only in fetal brain, but also in epileptic frontal cortex, and 12 week old early stage human. For a number of immune and/or nervous system-related and/or developmental disorders, substantially altered (increased or decreased) levels of Dendriac and/or Brainiac-3 gene expression can be detected in immune and/or nervous system tissue or other cells or bodily fluids (e.g., sera, plasma, urine, synovial fluid or spinal fluid) taken from an individual having such a disorder, relative to a “standard” Dendriac and/or Brainiac-3 gene expression level, that is, the Dendriac and/or Brainiac-3 expression levels in immune and/or nervous system tissues or bodily fluids from an individual not having the immune and/or nervous system disorder. Thus, the invention provides a diagnostic method useful during diagnosis of a immune and/or nervous system disorder, which involves measuring the expression level of the gene encoding the Dendriac and/or Brainiac-3 polypeptides in immune and/or nervous system tissue or other cells or body fluid from an individual and comparing the measured gene expression level with a standard Dendriac and/or Brainiac-3 gene expression level, whereby an increase or decrease in the gene expression level compared to the standard is indicative of an immune and/or nervous system disorder.

In particular, it is believed that certain tissues in mammals with cancer of the immune and nervous systems express significantly enhanced or reduced levels of the Dendriac and/or Brainiac-3 polypeptides and MRNA encoding the Dendriac and/or Brainiac-3 polypeptides when compared to a corresponding “standard” level. Further, it is believed that enhanced levels of the Dendriac and/or Brainiac-3 polypeptides can be detected in certain body fluids (e.g., sera, plasma, urine, and spinal fluid) from mammals with such a cancer when compared to sera from mammals of the same species not having the cancer.

Thus, the invention provides a diagnostic method useful during diagnosis of an immune and/or nervous system disorder, including cancers of these systems, which involves measuring the expression level of the gene encoding the Dendriac and/or Brainiac-3 polypeptides in immune and/or nervous system tissue or other cells or body fluid from an individual and comparing the measured gene expression level with a standard Dendriac and/or Brainiac-3 gene expression level, whereby an increase or decrease in the gene expression level compared to the standard is indicative of an immune and/or nervous system disorder.

Where a diagnosis of a disorder in the immune and/or nervous systems including diagnosis of a tumor, has already been made according to conventional methods, the present invention is useful as a prognostic indicator, whereby patients exhibiting enhanced or depressed Dendriac and/or Brainiac-3 gene expression will experience a worse clinical outcome relative to patients expressing the genes at a level nearer the standard level.

By “assaying the expression level of the genes encoding the Dendriac and/or Brainiac-3 polypeptides” is intended qualitatively or quantitatively measuring or estimating the level of the Dendriac and/or Brainiac-3 polypeptides or the level of the MRNA encoding the Dendriac and/or Brainiac-3 polypeptides in a first biological sample either directly (e.g., by determining or estimating absolute protein level or mRNA level) or relatively (e.g., by comparing to the Dendriac and/or Brainiac-3 polypeptide levels or mRNA levels in a second biological sample). Preferably, the Dendriac and/or Brainiac-3 polypeptides level or mRNA level in the first biological sample is measured or estimated and compared to a standard Dendriac and/or Brainiac-3 polypeptide level or mRNA level, the standard being taken from a second biological sample obtained from an individual not having the disorder or being determined by averaging levels from a population of individuals not having a disorder of the immune and/or nervous systems. As will be appreciated in the art, once a standard Dendriac and/or Brainiac-3 polypeptide level or mRNA level is known, it can be used repeatedly as a standard for comparison.

By “biological sample” is intended any biological sample obtained from an individual, body fluid, cell line, tissue culture, or other source which contains Dendriac and/or Brainiac-3 polypeptides or mRNA. As indicated, biological samples include body fluids (such as sera, plasma, urine, synovial fluid and spinal fluid) which contain free Dendriac and/or Brainiac-3 polypeptides, immune and/or nervous system tissue, and other tissue sources found to express complete or mature Dendriac and/or Brainiac-3 polypeptides or a Dendriac receptor or a Brainiac-3 receptor. Methods for obtaining tissue biopsies and body fluids from mammals are well known in the art. Where the biological sample is to include mRNA, a tissue biopsy is the preferred source.

The present invention is useful for diagnosis or treatment of various immune and/or nervous system-related disorders in mammals, preferably humans. A nonexclusive list of preferred mammals includes monkeys, apes, cats, dogs, cows, pigs, horses, rabbits, and humans. Humans are particularly preferred mammals. Such disorders include any disregulation of immune and/or nervous system cell and/or tissue function including, but not limited to Alzheimer's Disease, Parkinsons Disease, Huntington's Disease, Tourettes Syndrome, epilepsy, schizophrenia, mania, dementia, paranoia, obsessive compulsive disorder, panic disorder, learning disabilities, ALS, psychoses, autism, and altered behaviors, including disorders in feeding, sleep patterns, balance, and perception, neuronal survival; synapse formation; conductance; neural differentiation, autoimmunity, arthritis, leukemias, lymphomas, immunosuppression, immunity, humoral immunity, inflammatory bowel disease, myelosuppression, lymphoproliferative disorders, in the maintenance and differentiation of various hematopoietic lineages from early hematopoietic stem and committed progenitor cells, anemia, pancytopenia, leukopenia, thrombocytopenia or leukemia, bone marrow cell ex vivo culture, bone marrow transplantation, bone marrow reconstitution, radiotherapy or chemotherapy of neoplasia, asthma, immune deficiency diseases such as AIDS, rheumatoid arthritis, sepsis, acne, psoriasis, Grave's Disease, lymphocytic thyroiditis, hyperthyroidism, hypothyroidism, graft versus host reaction, graft versus host disease, transplant rejection, myelogenous leukemia, bone marrow fibrosis, and myeloproliferative disease, Huntingtons disease gene and other neurodegenerative diseases including spinocerebullar ataxia types I and III, dentatorubropallidoluysian and spinal bulbar muscular atrophy, and the like.

Total cellular RNA can be isolated from a biological sample using any suitable technique such as the single-step guanidinium-thiocyanate-phenol-chloroform method described by Chomczynski and Sacchi (Anal. Biochem. 162:156-159 (1987)). Levels of mRNA encoding the Dendriac and/or Brainiac-3 polypeptides are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Assaying Dendriac and/or Brainiac-3 polypeptide levels in a biological sample can occur using antibody-based techniques. For example, Dendriac and/or Brainiac-3 polypeptide expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting Dendriac and/or Brainiac-3 gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying Dendriac and/or Brainiac-3 polypeptide levels in a biological sample obtained from an individual, Dendriac and/or Brainiac-3 polypeptides can also be detected in vivo by imaging. Antibody labels or markers for in vivo imaging of Dendriac and/or Brainiac-3 polypeptides include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A Dendriac-specific antibody or antibody fragment, or a brainiac-3 -specific antibody or antibody fragment, which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain Dendriac and/or Brainiac-3 polypeptides. In vivo tumor imaging is described by Burchiel and coworkers (Chapter 13 in Tumor Imaging: The Radiochemical Detection of Cancer, Burchiel, S. W. and Rhodes, B. A., eds., Masson Publishing Inc. (1982)).

Treatment

As noted above, Dendriac and/or Brainiac-3 polynucleotides and polypeptides are useful for diagnosis of conditions involving abnormally high or low expression of Dendriac and/or Brainiac-3 activities. Given the cells and tissues where Dendriac and/or Brainiac-3 polypeptides are expressed as well as the activities modulated by Dendriac and/or Brainiac-3 polypeptides, it is readily apparent that a substantially altered (increased or decreased) level of expression of Dendriac and/or Brainiac-3 polypeptides in an individual compared to the standard or “normal” level produces pathological conditions related to the bodily system(s) in which Dendriac and/or Brainiac-3 polypeptides are expressed and/or is active.

It is well-known in the art that, in addition to a specific cellular function, cellular receptor molecules may also often be exploited by a virus as a means of initiating entry into a potential host cell. For example, it was recently discovered by Wu and colleagues (J. Exp. Med. 185:1681-1691 (1997)) that the cellular chemokine receptor CCR5 functions not only as a cellular chemokine receptor, but also as a receptor for macrophage-tropic human immunodeficiency virus (HIV)-1. In addition, RANTES, MIP-1α, and MIP-1β, which are agonists for the cellular chemokine receptor CCR5, inhibit entry of various strains of HIV-1 into susceptible cell lines (Cocchi, F., et al., Science 270:1811-1815 (1995)). Thus, the invention also provides a method of treating an individual exposed to, or infected with, a virus through the prophylactic or therapeutic administration of Dendriac and/or Brainiac-3 polypeptides, or an agonist or antagonist thereof, to block or disrupt the interaction of a viral particle with the Dendriac and/or Brainiac-3 receptors and, as a result, block the initiation or continuation of viral infectivity.

The Dendriac polypeptides of the present invention binds to the Dendriac receptor and, as such, is likely to block immune-tropic viral infections. Agonists and antagonists of the Dendriac-Dendriac Receptor interaction are also likely to interfere with immune-tropic viral infection. As a result, such an interaction is likely to interfere with the infectious life cycle of one or more immune-tropic viruses such as HIV-1, HIV-2, HTLV-III, HSV-1, HSV-2, and the like. In addition, the Brainiac-3 polypeptides of the present invention binds to the Brainiac-3 receptor and, as such, is likely to block neuro-tropic viral infections. Agonists and antagonists of the Brainiac-3-Brainiac-3 Receptor interaction are also likely to interfere with neuro-tropic viral infection. As a result, such an interaction is likely to interfere with the infectious life cycle of one or more neuro-tropic viruses such as HIV-1, HIV-2, HTLV-III, HSV-1, HSV-2, and the like

The ability of Dendriac and/or Brainiac-3 polypeptides of the present invention, or agonists or antagonists thereof, to prophylactically or therapeutically block viral infection may be easily tested by the skilled artisan. For example, Simmons and coworkers (Science 276:276-279 (1997)) and Arenzana-Seisdedos and colleagues (Nature 383:400 (1996)) each outline a method of observing suppression of HIV-1 infection by an antagonist of the CCR5 chemokine receptor and of the CC chemokine RANTES, respectively, in cultured peripheral blood mononuclear cells. Cells are cultured and infected with a virus, HIV-1 in both cases noted above. An agonist or antagonist of the CC chemokine or its receptor is then immediately added to the culture medium. Evidence of the ability of the agonist or antagonist of the chemokine or cellular receptor is determined by evaluating the relative success of viral infection at 3, 6, and 9 days postinfection.

Administration of a pharmaceutical composition comprising an amount of an isolated Dendriac and/or Brainiac-3 polypeptide, or an agonist or antagonist thereof, of the invention to an individual either infected with a virus or at risk for infection with a virus is performed as described below.

It will also be appreciated by one of ordinary skill that, since the Dendriac and/or Brainiac-3 polypeptides of the invention is a member of the Brainiac family, the mature secreted form of the polypeptide may be released in soluble form from the cells which express the Dendriac and/or Brainiac-3 polypeptides by proteolytic cleavage. Therefore, when the mature form of a Dendriac and/or Brainiac-3 polypeptide is added from an exogenous source to cells, tissues or the body of an individual, the polypeptide will exert its physiological activities on its target cells of that individual.

Therefore, it will be appreciated that conditions caused by a decrease in the standard or normal level of Dendriac and/or Brainiac-3 activity in an individual, particularly disorders of the immune and/or nervous systems, can be treated by administration of Dendriac and/or Brainiac-3 polypeptide (preferably in the form of the mature form of the polypeptide). Thus, the invention also provides a method of treatment of an individual in need of an increased level of Dendriac and/or Brainiac-3 activity comprising administering to such an individual a pharmaceutical composition comprising an amount of an isolated Dendriac and/or Brainiac-3 polypeptide of the invention, particularly a mature form of the Dendriac and/or Brainiac-3 polypeptides of the invention, effective to increase the Dendriac and/or Brainiac-3 polypeptide activity level in such an individual.

Formulations

The Dendriac and/or Brainiac-3 polypeptide composition will be formulated and dosed in a fashion consistent with good medical practice, taking into account the clinical condition of the individual patient (especially the side effects of treatment with Dendriac and/or Brainiac-3 polypeptides alone), the site of delivery of the Dendriac and/or Brainiac-3 polypeptide composition, the method of administration, the scheduling of administration, and other factors known to practitioners. The effective amount of Dendriac and/or Brainiac-3 polypeptide for purposes herein is thus determined by such considerations.

As a general proposition, the total pharmaceutically effective amount of Dendriac and/or Brainiac-3 polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Dendriac and/or Brainiac-3 polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the Dendriac and/or Brainiac-3 polypeptides of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

The Dendriac and/or Brainiac-3 polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U., et al., Biopolymers 22:547-556 (1983)), poly (2-hydroxyethyl methacrylate; Langer, R., et al., J. Biomed. Mater. Res. 15:167-277 (1981), and Langer, R., Chem. Tech. 12:98-105 (1982)), ethylene vinyl acetate (Langer, R., et al., Id.) or poly-D-(-)-3-hydroxybutyric acid (EP 133,988). Sustained-release Dendriac and/or Brainiac-3 polypeptide compositions also include liposomally entrapped Dendriac and/or Brainiac-3 polypeptide. Liposomes containing Dendriac and/or Brainiac-3 polypeptides are prepared by methods known in the art (DE 3,218,121; Epstein, et al., Proc. Natl. Acad. Sci. (USA) 82:3688-3692 (1985); Hwang, et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324). Ordinarily, the liposomes are of the small (about 200-800 Angstroms) unilamellar type in which the lipid content is greater than about 30 mol. percent cholesterol, the selected proportion being adjusted for the optimal Dendriac and/or Brainiac-3 polypeptide therapy.

For parenteral administration, in one embodiment, the Dendriac and/or Brainiac-3 polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the Dendriac and/or Brainiac-3 polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringers solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxarners, or PEG.

The Dendriac and/or Brainiac-3 polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of Dendriac and/or Brainiac-3 polypeptide salts.

Dendriac and/or Brainiac-3 polypeptide to be used for therapeutic administration must be sterile. Sterility is readily accomplished by filtration through sterile filtration membranes (e.g., 0.2 micron membranes). Therapeutic Dendriac and/or Brainiac-3 polypeptide compositions generally are placed into a container having a sterile access port, for example, an intravenous solution bag or vial having a stopper pierceable by a hypodermic injection needle.

Dendriac and/or Brainiac-3 polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Dendriac and/or Brainiac-3 polypeptide solution, and the resulting mixture is lyophilized. The infusion solution is prepared by reconstituting the lyophilized Dendriac and/or Brainiac-3 polypeptide using bacteriostatic water-for-injection (WFI).

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists -Assays and Molecules

The invention also provides a method of screening compounds to identify those which enhance or block the action of Dendriac and/or Brainiac-3 polypeptides on cells, such as its interaction with Dendriac and/or Brainiac-3 polypeptide-binding molecules such as receptor molecules. An agonist is a compound which increases the natural biological functions of Dendriac and/or Brainiac-3 polypeptides or which functions in a manner similar to Dendriac and/or Brainiac-3 polypeptides, while antagonists decrease or eliminate such functions.

In another aspect of this embodiment the invention provides a method for identifying a receptor protein or other ligand-binding protein which binds specifically to a Dendriac and/or Brainiac-3 polypeptide. For example, a cellular compartment, such as a membrane or a preparation thereof, may be prepared from a cell that expresses a molecule that binds Dendriac and/or Brainiac-3 polypeptides. The preparation is incubated with labeled Dendriac and/or Brainiac-3 polypeptide and complexes of Dendriac and/or Brainiac-3 polypeptide bound to the receptor or other binding protein are isolated and characterized according to routine methods known in the art. Alternatively, the Dendriac and/or Brainiac-3 polypeptide may be bound to a solid support so that binding molecules solubilized from cells are bound to the column and then eluted and characterized according to routine methods.

In the assay of the invention for agonists or antagonists, a cellular compartment, such as a membrane or a preparation thereof, may be prepared from a cell that expresses a molecule that binds Dendriac and/or Brainiac-3 polypeptide, such as a molecule of a signaling or regulatory pathway modulated by Dendriac and/or Brainiac-3 polypeptide. The preparation is incubated with labeled Dendriac and/or Brainiac-3 polypeptide in the absence or the presence of a candidate molecule which may be a Dendriac and/or Brainiac-3 polypeptide agonist or antagonist. The ability of the candidate molecule to bind the binding molecule is reflected in decreased binding of the labeled ligand. Molecules which bind gratuitously, i.e., without inducing the effects of Dendriac and/or Brainiac-3 polypeptide on binding the Dendriac and/or Brainiac-3 polypeptide-binding molecule, are most likely to be good antagonists. Molecules that bind well and elicit effects that are the same as or closely related to Dendriac and/or Brainiac-3 polypeptide are agonists.

Dendriac and/or Brainiac-3 polypeptide-like effects of potential agonists and antagonists may by measured, for instance, by determining activity of a second messenger system following interaction of the candidate molecule with a cell or appropriate cell preparation, and comparing the effect with that of Dendriac and/or Brainiac-3 polypeptides or molecules that elicit the same effects as Dendriac and/or Brainiac-3 polypeptide. Second messenger systems that may be useful in this regard include but are not limited to AMP guanylate cyclase, ion channel or phosphoinositide hydrolysis second messenger systems.

Another example of an assay for Dendriac and/or Brainiac-3 polypeptide antagonists is a competitive assay that combines Dendriac and/or Brainiac-3 polypeptides and a potential antagonist with membrane-bound Dendriac and/or Brainiac-3 polypeptide receptor molecules or recombinant Dendriac and/or Brainiac-3 polypeptide receptor molecules under appropriate conditions for a competitive inhibition assay. Dendriac and/or Brainiac-3 polypeptides can be labeled, such as by radioactivity, such that the number of Dendriac and/or Brainiac-3 polypeptide molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing Dendriac and/or Brainiac-3 polypeptide-induced activities, thereby preventing the action of Dendriac and/or Brainiac-3 polypeptides by excluding Dendriac and/or Brainiac-3 polypeptides from binding.

Other potential antagonists include antisense molecules. Antisense technology can be used to control gene expression through antisense DNA or RNA or through triple-helix formation. Antisense techniques are discussed in a number of studies (for example, Okano, J. Neurochem. 56:560 (1991); “Oligodeoxynucleotides as Antisense Inhibitors of Gene Expression.” CRC Press, Boca Raton, Fla. (1988)). Triple helix formation is discussed in a number of studies, as well (for instance, Lee, et al., Nucleic Acids Research 6:3073 (1979); Cooney, et al., Science 241:456 (1988); Dervan, et al., Science 251:1360 (1991)). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of Dendriac and/or Brainiac-3 polypeptide. The antisense RNA oligonucleotide hybridizes to the MRNA in vivo and blocks translation of the mRNA molecule into Dendriac and/or Brainiac-3 polypeptide. The oligonucleotides described above can also be delivered to cells such that the antisense RNA or DNA may be expressed in vivo to inhibit production of Dendriac and/or Brainiac-3 polypeptides.

The agonists and antagonists may be employed in a composition with a pharmaceutically acceptable carrier, e.g., as described above.

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a Dendriac and/or Brainiac-3 gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp (for a review of this technique, see Verma, et al., Human Chromosomes: A Manual Of Basic Techniques, Pergamon Press, New York (1988)).

Once a sequence has been mapped to a precise chromosomal location, the physical position of the sequence on the chromosome can be correlated with genetic map data. Such data are found, for example, on the World Wide Web (McKusick, V. Mendelian Inheritance In Man, available on-line through Johns Hopkins University, Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

Example 1: Expression and Purification of “His-tagged” Dendriac in E. coli

The novel pHE4 series of bacterial expression vectors, in particular, the pHE4-5 vector may be used for bacterial expression in this example. (QIAGEN, Inc., 9259 Eton Avenue, Chatsworth, Calif., 91311). pHE4-5/MPIFΔ23 vector plasmid DNA contains an insert which encodes another ORF. The construct was deposited with the American Type Culture Collection, 10801 University Boulevard, Manassas, Va. 20110-2209, on Sep. 30, 1997 and given Accession No. 209311. Using the Nde I and Asp 718 restriction sites flanking the irrelevant MPIF ORF insert, one of ordinary skill in the art could easily use current molecular biological techniques to replace the irrelevant ORF in the pHE4-5 vector with the Dendriac or Brainiac-3 ORF of the present invention.

The pHE4-5 bacterial expression vector includes a neomycin phosphotransferase gene for selection, an E. coli origin of replication, a T5 phage promoter sequence, two lac operator sequences, a Shine-Delgamo sequence, and the lactose operon repressor gene (lacIq). These elements are arranged such that an inserted DNA fragment encoding a polypeptide expresses that polypeptide with the six His residues (i.e., a “6× His tag”) covalently linked to the amino terminus of that polypeptide. The promoter and operator sequences of the pHE4-5 vector were made synthetically. Synthetic production of nucleic acid sequences is well known in the art (CLONETECH 95/96 Catalog, pages 215-216, CLONETECH, 1020 East Meadow Circle, Palo Alto, Calif. 94303).

The DNA sequence encoding the desired portion of the Dendriac polypeptide comprising the mature form of the Dendriac amino acid sequence is amplified from the deposited cDNA clone using PCR oligonucleotide primers which anneal to the amino terminal sequences of the desired portion of the Dendriac polypeptide and to sequences in the deposited construct 3to the cDNA coding sequence. Additional nucleotides containing restriction sites to facilitate cloning in the pHE4-5 vector are added to the 5′ and 3′ primer sequences, respectively.

For cloning the mature form of the Dendriac polypeptide, the 5′ primer has the sequence 5′ CAA TTG GAT CCC TTC CCC ACT ACA ATG TG 3′ (SEQ ID NO: 13) containing the underlined Bam HI restriction site followed by 18 nucleotides of the amino terminal coding sequence of the mature Dendriac sequence in SEQ ID NO:2 and SEQ ID NO:10. One of ordinary skill in the art would appreciate, of course, that the point in the protein coding sequence where the 5′ primer begins may be varied to amplify a DNA segment encoding any desired portion of the complete Dendriac polypeptide shorter or longer than the mature form of the polypeptide. The 3′ primer has the sequence 5′ GTA CGC AAG ATA ATG GCA TGT GGT GTT CC 3′ (SEQ ID NO: 14) containing the underlined Hin dIII restriction site followed by 17 nucleotides complementary to the 3end of the coding sequence of the Dendriac DNA sequence shown in FIGS. 1A, 1B, and 1C (and in SEQ ID NO:1 and in SEQ ID NO:9).

The amplified Dendriac DNA fragment and the vector pHE4-5 are digested with Bam HI and Hin dll and the digested DNAs are then ligated together. Insertion of the Dendriac DNA into the restricted pHE4-5 vector places the Dendriac polypeptide coding region downstream from the IPTG-inducible promoter and in-frame with an initiating AUG and the six histidine codons.

The skilled artisan appreciates that a similar approach could easily be designed and utilized to generate a pHE4-5-based bacterial expression construct for the expression of Brainiac-3 in E. coli. This would be done by designing PCR primers containing similar restriction endonuclease recognition sequences combined with gene-specific sequences for Brainiac-3 and proceeding as described for Dendriac above.

The ligation mixture is transformed into competent E. Coli cells using standard procedures such as those described by Sambrook and colleagues (Molecular Cloning: a Laboratory Manual, 2nd Ed.; Cold Spring Harbor Laboratory Press, Cold Spring Harbor, N.Y. (1989)). E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing Dendriac polypeptide, is available commercially (QIAGEN, Inc., supra). Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-β-D-thiogalactopyranoside (“IPTG”) is then added to a final concentration of 1 mM to induce transcription from the lac repressor sensitive promoter, by inactivating the laci repressor. Cells subsequently are incubated fuirther for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the Dendriac polypeptide is loaded onto a nickel-nitrilo-tri-acetic acid (“Ni-NTA”) affinity resin column (QIAGEN, Inc., supra). Proteins with a 6× His tag bind to the Ni-NTA resin with high affinity and can be purified in a simple one-step procedure (for details see: The QIAexpressionist, 1995, QIAGEN, Inc., supra). Briefly the supernatant is loaded onto the column in 6 M guanidine-HCl, pH 8, the column is first-washed with 10 volumes of 6 M guanidine-HCl, pH 8, then washed with 10 volumes of 6 M guanidine-HCl pH 6, and finally the Dendriac polypeptide is eluted with 6 M guanidine-HCl, pH 5.

The purified polypeptide is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins can be eluted by the addition of 250 mM immidazole. Immidazole is removed by a final dialyzing step against PBS or 50 mM sodium acetate pH 6 buffer plus 200 mM NaCl. The purified protein is stored at 40° C. or frozen at −80° C.

The following alternative method may be used to purify Dendriac polypeptide expressed in E. coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear nuxer.

The cells ware then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000× g centrifugation for 15 min., the pellet is discarded and the Dendriac polypeptide-containing supernatant is incubated at 4° C. overnight to allow further GuHCl extraction.

Following high speed centrifugation (30,000× g) to remove insoluble particles, the GuHCl solubilized protein is refolded by quickly mixing the GuHCI extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to frther purification steps.

To clarify the refolded Dendriac polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the Dendriac polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the Dendriac polypeptide (determined, for instance, by 16% SDS-PAGE) are then pooled.

The resultant Dendriac polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

The following alternative method may be used to purify Dendriac polypeptide expressed in E. coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells ware then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000× g centrifugation for 15 min., the pellet is discarded and the Dendriac polypeptide-containing supernatant is incubated at 4° C. overnight to allow further GuHCl extraction.

Following high speed centrifugation (30,000× g) to remove insoluble particles, the GuHCI solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded Dendriac polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the Dendriac polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the Dendriac polypeptide (determined, for instance, by 16% SDS-PAGE) are then pooled.

The resultant Dendriac polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

Example 2: Cloning and Expression of Dendriac polypeptide in a Baculovirus Expression System

In this illustrative example, the plasmid shuttle vector pA2 is used to insert the cloned DNA encoding complete polypeptide, including its naturally associated secretory signal (leader) sequence, into a baculovirus to express the mature Dendriac polypeptide, using standard methods as described by Summers and colleagues (A Manual of Methods for Baculovirus Vectors and Insect Cell Culture Procedures, Texas Agricultural Experimental Station Bulletin No. 1555 (1987)). This expression vector contains the strong polyhedrin promoter of the Autographa californica nuclear polyhedrosis virus (AcMNPV) followed by convenient restriction sites such as Bam HI, xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, by Luckow and coworkers (Virology 170:31-39 (1989)).

The cDNA sequence encoding the full-length Dendriac polypeptide in the deposited clones, including the AUG initiation codon and the naturally associated leader sequence shown in SEQ ID NO:2 and in SEQ ID NO:10, is amplified using PCR oligonucleotide primers corresponding to the 5′ and 3′ sequences of the gene. The 5′ primer has the sequence 5′ CGC GGA TCC GCC ATC ATG TCA CTG AGA TCC C 3′ (SEQ ID NO: 15) containing the underlined Bam HI restriction enzyme site, an efficient signal for initiation of translation in eukaryotic cells ((shown in italics); see, Kozak, M., J. Mol. Biol. 196:947-950 (1987)), followed by 16 nucleotides of the sequence of the complete Dendriac polypeptide shown in FIGS. 1A, 1B, and 1C and in SEQ ID NO:2 and SEQ ID NO:10, beginning with the AUG initiation codon. The 3′ primer has the sequence 5′ CAC TTA GGT ACC ATA ATG GCA TGT GGT GTT CC 3′ (SEQ ID NO:16) containing the underlined Asp 718 restriction site followed by 17 nucleotides complementary to the 3′ noncoding sequence in FIGS. 1A, 1B, and 1C (and in SEQ ID NO:2 and SEQ ID NO: 10).

The amplified fragment is isolated from a 1% agarose gel using a commercially available kit (“Geneclean, ” BIO 101 Inc., La Jolla, Calif.). The fragment then is digested with Bam HI and Asp 718 and again is purified on a 1% agarose gel. This fragment is designated herein F 1.

The plasmid is digested with the restriction enzymes Bam HI and Asp 718 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human Dendriac gene by digesting DNA from individual colonies using Bam HI and Asp 718 and then analyzing the digestion product by gel electrophoresis. The sequence of the cloned fragment is confirmed by DNA sequencing. This plasmid is designated herein pA2Dendriac.

Five μg of the plasmid pA2Dendriac is co-transfected with 1.0 μg of a commercially available linearized baculovirus DNA (“BaculoGold™ baculovirus DNA”, Pharmingen, San Diego, Calif.), using the lipofection method described by Felgner and colleagues (Proc. Nat. Acad. Sci. USA 84:7413-7417 (1987)). One μg of BaculoGold™ virus DNA and 5 μg of the plasmid pA2Dendriac are mixed in a sterile well of a microtiter plate containing 50 μl of serum-free Graces medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith (supra). An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Graces medium and the suspension containing the recombinant baculovirus is used to infect Sf9 cells seeded in 35 mm dishes. Four days later the supernatants of these culture dishes are harvested and then they are stored at 4° C. The recombinant virus is called V-Dendriac.

To verify the expression of the Dendriac gene Sf9 cells are grown in Grace's medium supplemented with 10% heat-inactivated FBS. The cells are infected with the recombinant baculovirus V-Dendriac at a multiplicity of infection (“MOI”) of about 2. If radiolabeled polypeptides are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The polypeptides in the supernatant as well as the intracellular polypeptides are analyzed by SDS-PAGE followed by autoradiography (if radiolabeled).

Microsequencing of the amino acid sequence of the amino terminus of purified polypeptide may be used to determine the amino terminal sequence of the mature form of the Dendriac polypeptide and thus the cleavage point and length of the naturally associated secretory signal peptide.

The skilled artisan appreciates that a similar approach could easily be designed and utilized to generate a pA2-based baculovirus expression construct for the expression of Brainiac-3 in insect cells. This would be done by designing PCR primers containing similar restriction endonuclease recognition sequences combined with gene-specific sequences for Brainiac-3 and proceeding as described for Dendriac above.

Example 3: Cloning and Expression of Dendriac in Mammalian Cell

A typical mammalian expression vector contains the promoter element, which mediates the initiation of transcription of mRNA, the polypeptide coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIV-1 and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded polypeptide. The DHFR (dihydrofolate reductase) marker is useful to develop cell lines that carry several hundred or even several thousand copies of the gene of interest. Another useful selection marker is the enzyme glutamine synthase (GS; Murphy, et al., Biochem J. 227:277-279 (1991); Bebbington, et al., Bio/Technology 10: 169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong promoter (LTR) of the Rous Sarcoma Virus (Cullen, et al., Mol. Cel. Biol. 5:438-447 (1985)) plus a fragment of the CMV-enhancer (Boshart, et al., Cell 41:521-530 (1985)). Multiple cloning sites, e.g., with the restriction enzyme cleavage sites Bam HI, Xba I and Asp 718, facilitate the cloning of the gene of interest. The vectors contain in addition the 3′ intron, the polyadenylation and termination signal of the rat preproinsulin gene.

Although the following Examples (3(a) and 3(b)) teach the subcloning and expression of Dendriac in mammalian cells, the skilled artisan appreciates that a similar approach could easily be designed and utilized to generate mammalian expression constructs for the expression of Brainiac-3 in mammalian cells. This would be done by designing PCR primers containing similar restriction endonuclease recognition sequences combined with gene-specific sequences for Brainiac-3 and proceeding as described for Dendriac below.

Example 3(a): Cloning and Expression in COS Cells

The expression plasmid, pDendriacHA, is made by cloning a portion of the cDNA encoding the mature form of the Dendriac polypeptide into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson and colleagues (Cell 37:767 (1984)). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the mature form of the Dendriac polypeptide is cloned into the polylinker region of the vector so that recombinant polypeptide expression is directed by the CMV promoter. The plasmid construction strategy is as follows. The Dendriac cDNA of the deposited clone is amplified using primers that contain convenient restriction sites, much as described above for construction of vectors for expression of Dendriac in E. coli. Suitable primers include the following, which are used in this example. The 5′ primer, containing the underlined Bam HI site, a Kozak sequence (in italics), an AUG start codon, and 16 nucleotides of the 5′ coding region of the mature Dendriac polypeptide, has the following sequence: 5′ CGC GGA TCC GCC ATC ATG TCA CTG AGA TCC C 3′ (SEQ ID NO:15). The 3′ primer, containing the underlined Asp 718 and 17 of nucleotides complementary to the 3′ coding sequence immediately before the stop codon, has the following sequence: 5′ CAC TTA GGT ACC ATA ATG GCA TGT GGT GTT CC 3′ (SEQ ID NO:16).

The PCR amplified DNA fragment and the vector, pcDNAI/Amp, are digested with Bam HI and Asp 718 and then ligated. The ligation mixture is transformed into E. coli strain SURE (Stratagene Cloning Systems, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the complete Dendriac polypeptide.

For expression of recombinant Dendriac, COS cells are transfected with an expression vector, as described above, using DEAE-dextran, as described, for instance, by Sambrook and coworkers (Molecular Cloning: a Laboratory Manual, Cold Spring Laboratory Press, Cold Spring Harbor, N.Y. (1989)). Cells are incubated under conditions for expression of Dendriac by the vector.

Expression of the Dendriac-HA fusion polypeptide is detected by radiolabeling and immunoprecipitation, using methods described in, for example Harlow and colleagues (Antibodies: A Laboratory Manual, 2nd Ed.; Cold Spring Harbor Laboratory Press, Cold Spring Harbor, N.Y. (1988)). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson and colleagues (supra). Polypeptides are precipitated from the cell lysate and from the culture media using an HA-specific monoclonal antibody. The precipitated polypeptides then are analyzed by SDS-PAGE and autoradiography. An expression product of the expected size is seen in the cell lysate, which is not seen in negative controls.

Example 3(b): Cloning and Expression in CHO Cells

The vector pC4 is used for the expression of Dendriac polypeptide. Plasmid pC4 is a derivative of the plasmid pSV2-dhfr (ATCC Accession No. 37146). The plasmid contains the mouse DHFR gene under control of the SV40 early promoter. Chinese hamster ovary or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., et al., J. Biol. Chem. 253:1357-1370 (1978); Hamlin, J. L. and Ma, C. Biochem. etBiophys. Acta, 1097:107-143 (1990); Page, M. J. and Sydenham, M. A. Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Mol. Cell. Biol. 5:438-447 (1985)) plus a fragment isolated from the enhancer of the immediate early gene of human cytomegalovirus (CMV; Boshart, et al., Cell 41:521-530 (1985)). Downstream of the promoter are the following single restriction enzyme cleavage sites that allow the integration of the genes: Bam HI, Xba I, and Asp 718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the Dendriac polypeptide in a regulated way in mammalian cells (Gossen, M., and Bujard, H. Proc. Natl. Acad. Sci. USA 89:5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes Bam HI and Asp 718 and then dephosphorylated using calf intestinal phosphates by procedures known in the art. The vector is then isolated from a 1% agarose gel.

The DNA sequence encoding the complete Dendriac polypeptide is amplified using PCR oligonucleotide primers corresponding to the 5′ and 3′ sequences of the desired portion of the gene. The 5′ primer containing the underlined Bam HI site, a Kozak sequence, an AUG start codon, and 16 nucleotides of the 5′ coding region of the complete Dendriac polypeptide, has the -following sequence: 5′ CGC GGA TCC GCC ATC ATG TCA CTG AGA TCC C 3′ (SEQ ID NO:15). The 3′ primer, containing the underlined Asp 718 and 17 of nucleotides complementary to the 3′ coding sequence immediately before the stop codon as shown in FIG. 1 (SEQ ID NO:1), has the following sequence: 5′ CAC TTA GGT ACC ATA ATG GCA TGT GGT GTT CC 3′ (SEQ ID NO:16).

The amplified fragment is digested with the endonucleases Bam HI and Asp 718 and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner, et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

Example 4: Tissue distribution of Dendriac and/or Brainiac-3 mRNA expression

Northern blot analysis is carried out to examine Dendriac and/or Brainiac-3 gene expression in human tissues, using methods described by, among others, Sambrook and colleagues (supra). A cDNA probe containing the entire nucleotide sequence of the Dendriac or the Brainiac-3 polypeptide (SEQ ID NO:1) is labeled with ³²p using the rediprime™ DNA labeling system (Amersham Life Science), according to manufacturer's instructions. After labeling, the probe is purified using a CHROMA SPIN-100™ column 20 (Clontech Laboratories, Inc.), according to manufacturers protocol number PT1200-1. The purified labeled probe is then used to examine various human tissues for Dendriac and/or Brainiac-3 mRNA.

Multiple Tissue Northern (MTN) blots containing various human tissues (H) or human immune system tissues (IM) are obtained from Clontech and are examined with the labeled probe using ExpressHyb™ hybridization solution (Clontech) according to manufacturers protocol number PT1190-1. Following hybridization and washing, the blots are mounted and exposed to film at −70° C. overnight, and films developed according to standard procedures.

Results of Northern blot experiments performed essentially as described above to examine expression of Dendriac mRNA include the following. Using Northern blot analysis, the Dendriac message was abundantly detected in brain, kidney, pancreas, testis, fetal liver, and thyroid. In addition, Northern blot experiments showed lower, but clear, levels of expression of Dendriac in the following tissues: lung, liver, amygdala, caudate nucleus, corpus callosum, hippocampus, whole brain, substantia nigra, subthalamic nucleus, thalamus, adrenal cortex, small intestine, stomach, spleen, lymph node, and colorectal adenocarcinoma SW480. Finally, very low levels of Dendriac expression were observed by Northern blot in the following tissues: prostate, uterus, lung carcinoma A549, and melanoma G361. Further, the Brainiac-3 message was abundantly detected in fetal brain and fetal kidney, and detected at lower, but clear, levels of expression in the lung and liver. In addition, the Brainiac-3 Northern blot expression studies identify an approximately 1.35 kb band in all positive tissues, an approximately 2.0 kb band in fetal kidney and fetal brain, and an approximately 4.0 kb band in fetal brain.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

Further, the Sequence Listing submitted herewith, and the Sequence Listings submitted with U.S. Provisional Application Ser. No. 60/108,928, filed on Nov. 17, 1998; U.S. Provisional Application Ser. No. 60/068,006, filed on Dec. 18, 1997; and U.S. Provisional Application Ser. No. 60/077,687, filed on Mar. 12, 1998 (to each of which the present application claims benefit of the filing dates under 35 U.S.C. § 119(e)), in both computer and paper forms are hereby incorporated by reference in their entireties.

What is claimed is:

1. An isolated nucleic acid molecule comprising a polynucleotide having a nucleotide sequence at least 95% identical to a sequence selected from the group consisting of:
(a) a nucleotide sequence encoding the Dendriac polypeptide having the complete amino acid sequence in SEQ ID NO:2 and SEQ ID NO:10 (i.e., positions −25 to 294 of SEQ ID NO:2 and SEQ ID NO:10);
(b) a nucleotide sequence encoding the Dendriac polypeptide having the complete amino acid sequence in SEQ ID NO:2 and SEQ ID NO: 10 excepting the N-terminal methionine (i.e., positions −24-294 of SEQ ID NO:2 and SEQ ID NO:10);
(c) a nucleotide sequence encoding the predicted mature Dendriac polypeptide having the amino acid sequence at positions 1-294 in SEQ ID NO:2 and SEQ ID NO: 10;
(d) a nucleotide sequence encoding the Dendriac polypeptide having the complete amino acid sequence encoded by the cDNA clone contained in ATCC Deposit No. 203056 and in ATCC Deposit No. 209056;
(e) a nucleotide sequence encoding the Dendriac polypeptide having the complete amino acid sequence excepting the N-terminal methionine encoded by the cDNA clone contained in ATCC Deposit No. 203056 and in ATCC Deposit No. 209056;
(f) a nucleotide sequence encoding the mature Dendriac polypeptide having the amino acid sequence encoded by the cDNA clone contained in ATCC Deposit No. 203056 and in ATCC Deposit No. 209056; and
(g) a nucleotide sequence encoding the Brainiac-3 polypeptide having the complete amino acid sequence in SEQ ID NO:4 and SEQ ID NO:12 (i.e., positions −28 to 324 of SEQ ID NO:4);
(h) a nucleotide sequence encoding the Brainiac-3 polypeptide having the complete amino acid sequence in SEQ ID NO:4 and SEQ ID NO:12 excepting the N-terminal methionine (i.e., positions −27-324 of SEQ ID NO:4 and SEQ ID NO:12);
(i) a nucleotide sequence encoding the predicted mature Brainiac-3 polypeptide having the amino acid sequence at positions 1-324 in SEQ ID NO:4 and SEQ ID NO:12;
(j) a nucleotide sequence encoding the Brainiac-3 polypeptide having the complete amino acid sequence encoded by the cDNA clone contained in ATCC Deposit No. 203451 and in ATCC Deposit No. 209463;
(k) a nucleotide sequence encoding the Brainiac-3 polypeptide having the complete amino acid sequence excepting the N-terminal methionine encoded by the cDNA clone contained in ATCC Deposit No. 203451 and in ATCC Deposit No. 209463;
(l) a nucleotide sequence encoding the mature Brainiac-3 polypeptide having the amino acid sequence encoded by the cDNA clone contained in ATCC Deposit No. 203451 and in ATCC Deposit No. 209463; and
(m) a nucleotide sequence complementary to any of the nucleotide sequences in (a), (b), (c), (d), (e), (f), (g), (h), (i), (j), (k) or (l), above.

2. The nucleic acid molecule of claim 1 wherein said polynucleotide has the complete nucleotide sequence in FIGS. 1A, 1B, and 1C (SEQ ID NO:1), in SEQ ID NO:9, in FIGS. 2A and 2B (SEQ ID NO:3) or in SEQ ID NO: 11.

3. The nucleic acid molecule of claim 1 wherein said polynucleotide has the nucleotide sequence in FIGS. 1A, 1B, and 1C (SEQ ID NO: 1) or in SEQ ID NO:9 encoding the Dendriac polypeptide having the amino acid sequence in positions −25 to 294 of SEQ ID NO:2 and SEQ ID NO:10.

4. The nucleic acid molecule of claim 1 wherein said polynucleotide has the nucleotide sequence in FIGS. 2A and 2B (SEQ ID NO:3) or in SEQ ID NO: 11 encoding the Brainiac-3 polypeptide having the amino acid sequence in positions −28 to 324 of SEQ ID NO:4 and SEQ ID NO:12.

5. The nucleic acid molecule of claim 1 wherein said polynucleotide has the nucleotide sequence in FIGS. 1A, 1B, and 1C (SEQ ID NO:1) or SEQ ID NO:9 encoding the Dendriac polypeptide having the amino acid sequence in positions −24 to 294 of SEQ ID NO:2 and SEQ ID NO:10.

6. The nucleic acid molecule of claim 1 wherein said polynucleotide has the nucleotide sequence in FIGS. 2A and 2B (SEQ ID NO:3) or SEQ ID NO:11 encoding the Brainiac-3 polypeptide having the amino acid sequence in positions −27 to 324 of SEQ ID NO:4 and SEQ ID NO:12.

7. The nucleic acid molecule of claim 1 wherein said polynucleotide has the nucleotide sequence in FIGS. 1A, 1B, and 1C (SEQ ID NO: 1) or SEQ ID NO:9 encoding the mature form of the Dendriac polypeptide having the amino acid sequence from about 1 to about 294 in SEQ ID NO:2 and SEQ ID NO:10.

8. The nucleic acid molecule of claim 1 wherein said polynucleotide has the nucleotide sequence in FIGS. 2A and 2B (SEQ ID NO:3) encoding the mature form of the Brainiac-3 polypeptide having the amino acid sequence from about 1 to about 324 in SEQ ID NO:4 and SEQ ID NO: 12.

9. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n¹-294 of SEQ ID NO:2 and SEQ ID NO:10, where n¹ is an integer in the range of −25 through 56;
(b) a nucleotide sequence encoding a polypeptide comprising the amino acid sequence of residues −25-m¹ of SEQ ID NO:2 and SEQ ID NO: 10, where m¹ is an integer in the range of 292 through 294;
(c) a nucleotide sequence encoding a polypeptide having the amino acid sequence consisting of residues n¹-m¹ of SEQ ID NO:2 and SEQ ID NO:10, where n and m are integers as defined respectively in (a) and (b) above;
(d) a nucleotide sequence encoding a polypeptide consisting of a portion of the complete Dendriac amino acid sequence encoded by the cDNA clone contained in ATCC Deposit Nos. 203056 and 209627 wherein said portion excludes from 1 to about 81 amino acids from the amino terminus of said complete amino acid sequence encoded by the cDNA clone contained in ATCC Deposit Nos. 203056 and 209627;
(e) a nucleotide sequence encoding a polypeptide consisting of a portion of the complete Dendriac amino acid sequence encoded by the cDNA clone contained in ATCC Deposit Nos. 203056 and 209627 wherein said portion excludes from 1 to about 3 amino acids from the carboxy terminus of said complete amino acid sequence encoded by the cDNA clone contained in ATCC Deposit Nos. 203056 and 209627;
(f) a nucleotide sequence encoding a polypeptide consisting of a portion of the complete Dendriac amino acid sequence encoded by the cDNA clone contained in ATCC Deposit Nos. 203056 and 209627 wherein said portion include a combination of any of the amino terminal and carboxy terminal deletions in (d) and (e), above;
(g) a nucleotide sequence encoding a polypeptide comprising the amino acid sequence of residues n²-324 of SEQ ID NO:4 and SEQ ID NO:12, where n² is an integer in the range of −28 through 80;
(h) a nucleotide sequence encoding a polypeptide comprising the amino acid sequence of residues −28-m² of SEQ ID NO:4 and SEQ ID NO:12, where m² is an integer in the range of 316 through 324;
(i) a nucleotide sequence encoding a polypeptide having the amino acid sequence consisting of residues n²-m² of SEQ ID NO:4 and SEQ ID NO: 12, where n² and m² are integers as defined respectively in (a) through (d) above;
a nucleotide sequence encoding a polypeptide consisting of a portion of the complete Brainiac-3 amino acid sequence encoded by the cDNA clone contained in ATCC Deposit Nos. 203451 and 209463 wherein said portion excludes from 1 to about 108 amino acids from the amino terminus of said complete amino acid sequence encoded by the cDNA clone contained in ATCC Deposit Nos. 203451 and 209463;
(k) a nucleotide sequence encoding a polypeptide consisting of a portion of the complete Brainiac-3 amino acid sequence encoded by the cDNA clone contained in ATCC Deposit Nos. 203451 and 209463 wherein said portion excludes from 1 to about 8 amino acids from the carboxy terminus of said complete amino acid sequence encoded by the cDNA clone contained in ATCC Deposit Nos. 203451 and 209463; and
(l) a nucleotide sequence encoding a polypeptide consisting of a portion of the complete Brainiac-3 amino acid sequence encoded by the cDNA clone contained in ATCC Deposit Nos. 203451 and 209463 wherein said portion include a combination of any of the amino terminal and carboxy terminal deletions in (d) and (e), above.

10. The nucleic acid molecule of claim 1 wherein said polynucleotide has the complete nucleotide sequence of the cDNA clone contained in ATCC Deposit No. 203056, in ATCC Deposit No. 209627, in ATCC Deposit No. 203451 or in ATCC Deposit No. 209463.

11. The nucleic acid molecule of claim 1 wherein said polynucleotide has the nucleotide sequence encoding the Dendriac or Brainiac-3 polypeptides having the complete amino acid sequence excepting the N-terminal methionine encoded by the cDNA clones contained in ATCC Deposit No. 203056, in ATCC Deposit No. 209627, in ATCC Deposit No. 203451 or in ATCC Deposit No. 209463.

12. The nucleic acid molecule of claim 1 wherein said polynucleotide has the nucleotide sequence encoding the mature form of the Dendriac or Brainiac-3 polypeptide having the amino acid sequence encoded by the cDNA clone contained in ATCC Deposit No. 203056, in ATCC Deposit No. 209627, in ATCC Deposit No. 203451 or in ATCC Deposit No. 209463.

13. An isolated nucleic acid molecule comprising a polynucleotide which hybridizes under stringent hybridization conditions to a polynucleotide having a nucleotide sequence identical to a nucleotide sequence in (a), (b), (c), (d), (e), (f), (g), (h), (i), (j), (k), (l) or (m) of claim 1 wherein said polynucleotide which hybridizes does not hybridize under stringent hybridization conditions to a polynucleotide having a nucleotide sequence consisting of only A residues or of only T residues.

14. An isolated nucleic acid molecule comprising a polynucleotide which encodes the amino acid sequence of an epitope-bearing portion of a Dendriac or Brainiac-3 polypeptide having an amino acid sequence in (a), (b), (c), (d), (e), (f), (g), (h), (i), (j), (k), (l) or (m) of claim 1.

15. The isolated nucleic acid molecule of claim 14, which encodes an epitope-bearing portion of a Dendriac polypeptide wherein the amino acid sequence of said portion is selected from the group of sequences in SEQ ID NO:2 and SEQ ID NO:10 consisting of: about Leu-30 to about His-38; from about His-50 to about Ala-59; from about Trp-64 to about Trp-70; from about Met-208 to about Val-213; from about Lys-224 to about Asp-230; from about Ile-246 to about Leu-252; and from about Gly-273 to about Glu-278.

16. The isolated nucleic acid molecule of claim 14, which encodes an epitope-bearing portion of a Brainiac-3 polypeptide wherein the amino acid sequence of said portion is selected from the group of sequences in SEQ ID NO:4 and SEQ ID NO:12 consisting of: about Lys-1 to about Ser-12; about Pro-16 to about Pro-25; about Leu-36 to about Arg-41; about Pro-58 to about Asp-64; about Ser-73 to about Ile-84; about Thr-87 to about Pro-97; about Leu-161 to about Val-167; about Asp-179 to about Gln-185; about Leu-197 to about Lys-203; about Gly-275 to about Asp-284; and Arg-296 to about Glu-301.

17. A method for making a recombinant vector comprising inserting an isolated nucleic acid molecule of claim 1 into a vector.

18. A recombinant vector produced by the method of claim 17.

19. A method of making a recombinant host cell comprising introducing the recombinant vector of claim 18 into a host cell.

20. A recombinant host cell produced by the method of claim 19.

21. A recombinant method for producing a Dendriac or Brainiac-3 polypeptide, comprising culturing the recombinant host cell of claim 20 under conditions such that said polypeptide is expressed and recovering said polypeptide.

22. An isolated Dendriac and Brainiac-3 polypeptide comprising an amino acid sequence at least 95% identical to a sequence selected from the group consisting of:
(a) the amino acid sequence of the full-length Dendriac polypeptide having the complete amino acid sequence shown in SEQ ID NO:2 and SEQ ID NO:10 (i.e., positions −25 through 294 of SEQ ID NO:2 and SEQ ID NO:10);
(b) the amino acid sequence of the full-length Dendriac polypeptide having the complete amino acid sequence shown in SEQ ID NO:2 and SEQ ID NO:10 excepting the N-terminal methionine (i.e., positions −24-294 of SEQ ID NO:2 and SEQ ID NO:10);
(c) the amino acid sequence of the predicted mature Dendriac polypeptide having the amino acid sequence at positions 1-294 in SEQ ID NO:2 and SEQ ID NO:10;
(d) the complete amino acid sequence encoded by the cDNA clone contained in the ATCC Deposit No. 203056 and in ATCC Deposit No. 209627;
(e) the complete amino acid sequence excepting the N-terminal methionine encoded by the cDNA clone contained in the ATCC Deposit No. 203056 and in ATCC Deposit No. 209627;
(f) the complete amino acid sequence of the predicted mature Dendriac polypeptide encoded by the cDNA clone contained in the ATCC Deposit No. 203056 and in ATCC Deposit No. 209627;
(g) the amino acid sequence of the full-length Brainiac-3 polypeptide having the complete amino acid sequence shown in SEQ ID NO:4 and SEQ ID NO:12 (i.e., positions −28 through 324 of SEQ ID NO:4 and SEQ ID NO:12);
(h) the amino acid sequence of the full-length Brainiac-3 polypeptide having the complete amino acid sequence shown in SEQ ID NO:4 and SEQ ID NO:12 excepting the N-terminal methionine (i.e., positions −27 through 324 of SEQ ID NO:4 and SEQ ID NO:12);
(i) the amino acid sequence of the predicted mature Brainiac-3 polypeptide having the amino acid sequence at positions 1 through 324 in SEQ ID NO:4 and SEQ ID NO:12;
(j) the complete amino acid sequence encoded by the cDNA clone contained in the ATCC Deposit No. 203451 and in ATCC Deposit No. 209463;
(k) the complete amino acid sequence excepting the N-terminal methionine encoded by the cDNA clone contained in the ATCC Deposit No. 203451 and in ATCC Deposit No. 209463; and
(l) the complete amino acid sequence of the predicted mature Brainiac-3 polypeptide encoded by the cDNA clone contained in the ATCC Deposit No. 203451 and in ATCC Deposit No. 209463.

23. An isolated polypeptide comprising an epitope-bearing portion of the Dendriac protein, wherein said portion is selected from the group consisting of: a polypeptide comprising amino acid residues from about Leu-30 to about His-38 of SEQ ID NO:2 and SEQ ID NO:10; a polypeptide comprising amino acid residues from about His-50 to about Ala-59 of SEQ ID NO:2 and SEQ ID NO:10; a polypeptide comprising amino acid residues from about Trp-64 to about Trp-70 of SEQ ID NO:2 and SEQ ID NO:10; a polypeptide comprising amino acid residues from about Met-208 to about Val-213 of SEQ ID NO:2 and SEQ ID NO:10; a polypeptide comprising amino acid residues from about Lys-224 to about Asp-230 of SEQ ID NO:2 and SEQ ID NO: 10; a polypeptide comprising amino acid residues from about Ile-246 to about Leu-252 of SEQ ID NO:2 and SEQ ID NO:10; and a polypeptide comprising amino acid residues from about Gly-273 to about Glu-278 of SEQ ID NO:2 and SEQ ID NO:10.

24. An isolated polypeptide comprising an epitope-bearing portion of the Brainiac-3 protein, wherein said portion is selected from the group consisting of: a polypeptide comprising amino acid residues from about Lys-l to about Ser-12 in SEQ ID NO:4 and SEQ ID NO:12; a polypeptide comprising amino acid residues from about Pro-16 to about Pro-25 in SEQ ID NO:4 and SEQ ID NO:12; a polypeptide comprising amino acid residues from about Leu-36 to about Arg-41 in SEQ ID NO:4 and SEQ ID NO:12; a polypeptide comprising amino acid residues from about Pro-58 to about Asp-64 in SEQ ID NO:4 and SEQ ID NO:12; a polypeptide comprising amino acid residues from about Ser-73 to about Ile-84 in SEQ ID NO:4 and SEQ ID NO: 12; a polypeptide comprising amino acid residues from about Thr-87 to about Pro-97 in SEQ ID NO:4 and SEQ ID NO:12; a polypeptide comprising amino acid residues from about Leu-161 to about Val-167 in SEQ ID NO:4 and SEQ ID NO:12; a polypeptide comprising amino acid residues from about Asp-179 to about Gln-185 in SEQ ID NO:4 and SEQ ID NO:12; a polypeptide comprising amino acid residues from about Leu-197 to about Lys-203 in SEQ ID NO:4 and SEQ ID NO:12; a polypeptide comprising amino acid residues from about Gly-275 to about Asp-284 in SEQ ID NO:4 and SEQ ID NO:12; and a polypeptide comprising amino acid residues from about Arg-296 to about Glu-301 in SEQ ID NO:4 and SEQ ID NO:12.

25. An isolated antibody that binds specifically to a Dendriac or Brainiac-3 polypeptide of claim 22.

26. An isolated nucleic acid molecule comprising a polynucleotide having a sequence at least 95% identical to a sequence selected from the group consisting of:
(a) the nucleotide sequence of SEQ ID NO:6;
(b) the nucleotide sequence of SEQ ID NO:7;
(c) the nucleotide sequence of SEQ ID NO:8;
(d) the nucleotide sequence of a portion of the sequence shown in FIGS. 2A and 2B (SEQ ID NO:3) wherein said portion comprises at least 50 contiguous nucleotides from nucleotide 150 to 650;
(e) a nucleotide sequence complementary to any of the nucleotide sequences in (a), (b), (c), and (d) above.
